SEQUENCE LISTING (10 sequences)

SEQ ID NO: 1 - DNA, 21 bp, synthetic construct
  source 1..21: mol_type = other DNA
ggctgagactaacccagaaac

SEQ ID NO: 2 - DNA, 21 bp, synthetic construct
  source 1..21: mol_type = other DNA
gaatgaaggtggctgctatga

SEQ ID NO: 3 - DNA, 20 bp, synthetic construct
  source 1..20: mol_type = other DNA
tgtgttcatccccagaaccg

SEQ ID NO: 4 - DNA, 20 bp, synthetic construct
  source 1..20: mol_type = other DNA
gggtacagttccttggagcc

SEQ ID NO: 5 - DNA, 23 bp, synthetic construct
  source 1..23: mol_type = other DNA
tgattatggacaggactgaaaga

SEQ ID NO: 6 - DNA, 23 bp, synthetic construct
  source 1..23: mol_type = other DNA
gcaggtcagcaaagaacttatag

SEQ ID NO: 7 - DNA, 20 bp, synthetic construct
  source 1..20: mol_type = other DNA
attccacccatggcaaattc

SEQ ID NO: 8 - DNA, 21 bp, synthetic construct
  source 1..21: mol_type = other DNA
ggatctcgctcctggaagatg

SEQ ID NO: 9 - RNA, 20 nt, synthetic construct
  source 1..20: mol_type = other RNA
agtatgccgtggatgaactg

SEQ ID NO: 10 - RNA, 20 nt, synthetic construct
  source 1..20: mol_type = other RNA
cagacccaacaagaagacca

(19) United States
(12) Patent Application Publication
(10) Pub. No.: US 2025/0179166 A1
(43) Pub. Date: Jun. 5, 2025

(54) TREATMENT OF BRAIN METASTASIS

(71) Applicant: Ramot at Tel-Aviv University Ltd., Tel-Aviv (IL)

(72) Inventors: Ronit SATCHI-FAINARO, Tel Aviv (IL); Sabina POZZI, Tel Aviv (IL); Sahar ISRAELI DANGOOR, Tel Aviv (IL)

(73) Assignee: Ramot at Tel-Aviv University Ltd., Tel-Aviv (IL)

(21) Appl. No.: 19/038,800

(22) Filed: Jan. 28, 2025

Related U.S. Application Data
(63) Continuation of application No. PCT/IL2023/050782, filed on Jul. 28, 2023.
(60) Provisional application No. 63/392,934, filed on Jul. 28, 2022.

(51) Int. Cl. C07K 16/24; A61K 31/416; A61K 31/506; A61K 31/517; A61K 31/53; A61K 31/537; A61K 31/55; A61K 31/7088; A61P 35/04; C07K 16/28

(52) U.S. Cl. CPC C07K 16/24; A61K 31/416; A61K 31/506; A61K 31/517; A61K 31/53; A61K 31/537; A61K 31/55; A61K 31/7088; A61P 35/04; C07K 16/2866

(57) ABSTRACT

A method of activating an immune response against brain metastasis in a subject in need thereof is provided. The method comprising administering to the subject a therapeutically effective amount of an inhibitor of an MCP-1/CCR2/CCR4 axis, thereby activating the immune response against brain metastasis in the subject.

### RELATED APPLICATIONS

This application is a Continuation of PCT Patent Application No. PCT/IL2023/050782 having International filing date of Jul. 28, 2023, which claims the benefit of priority under 35 USC § 119(e) of U.S. Provisional Patent Application No. 63/392,934 filed on Jul. 28, 2022. The contents of the above applications are all incorporated by reference as if fully set forth herein in their entirety.

The work leading to this invention has received funding from the European Union ERC Program, H2020-EU.1.1.—EXCELLENT SCIENCE—European Research Council (ERC), Jan. 4, 2019-31 Mar. 2024 under grant agreement no 835227.

### SEQUENCE LISTING STATEMENT

The XML file, entitled 102478SequenceListing.xml, created on Dec. 23, 2024, comprising 9,459 bytes, submitted concurrently with the filing of this application is incorporated herein by reference.

### FIELD AND BACKGROUND OF THE INVENTION

The present invention, in some embodiments thereof, relates to treatment of brain metastasis.

A brain metastasis is a cancer that has metastasized (spread) to the brain from another location in the body and is therefore considered a secondary brain tumor. The metastasis typically shares a cancer cell type with the original site of the cancer. Metastasis is the most common cause of brain cancer, as primary tumors that originate in the brain are less common. The most common sites of primary cancer which metastasize to the brain are lung, breast, colon, kidney, and skin cancer, e.g., cutaneous melanoma. Brain metastases can occur in patients months or even years after their original cancer is treated. Brain metastases have a poor prognosis for cure.

Cutaneous melanoma is the deadliest of all skin cancers, especially due to its tendency to invade and develop metastases at distant sites. It is the third primary malignancy after lung and breast cancers that preferentially colonizes the brain, with an incidence of brain metastasis development of 40% to 50% in patients with melanoma stage IV (although the incidence post mortem is 70-90%) (1, 2). Without any therapeutic intervention, metastatic lesions lead to a median survival of less than 7 months. The current standard treatments, including radiation, chemotherapy and targeted therapies, raised the overall survival of almost 85% of patients diagnosed with melanoma brain metastases (MBM) from 12 to 14 months (3). Recent advances in immunotherapy raised the overall survival to 2 years, however, heterogeneity in immune responses and resistance to treatments are frequently observed due to distinct adaptive and innate immune cell infiltration into the tumor (3-6).

The brain microenvironment (BME) represents the first line of reaction in favor or against the tumor due to its dual ability to generate an immune-stimulatory or immunosuppressive niche. This will ultimately determine the establishment and growth of MBM (5, 7). Among the BME-resident cells, astrocytes are responsible for the maintenance of the brain homeostasis (8). Subsequent to melanoma brain colonization, alterations in astrocyte morphology and protein expression are generally observed and considered as hallmarks of neuroinflammation (9, 10). Indeed, the activation of astrocytes induces the upregulation of glial fibrillary acid protein (GFAP), secretion of growth factors, fatty acids, and brain damage-related factors. Altogether, these changes allow them to sustain and foster the growth and development of primary and secondary brain lesions (11, 12).

Additional background art includes:
Patent Application Publication No. US20110033521.

### SUMMARY OF THE INVENTION

According to an aspect of some embodiments of the present invention there is provided a method of treating brain metastasis in a subject in need thereof, the method comprising administering to the subject a therapeutically effective amount of an inhibitor of an MCP-1/CCR2/CCR4 axis, thereby treating the brain metastasis in the subject.

According to an aspect of some embodiments of the present invention there is provided a method of activating an immune response against brain metastasis in a subject in need thereof, the method comprising administering to the subject a therapeutically effective amount of an inhibitor of an MCP-1/CCR2/CCR4 axis, thereby activating the immune response against brain metastasis in the subject.

According to some embodiments of the invention, the activating the immune response is by at least one of:
(i) inducing polarization or activation of immunosuppressive immune cells (e.g., macrophages) from a pro-tumorogenic anti-inflammatory phenotype towards an immune-potentiator pro-inflammatory anti-tumorogenic phenotype; and
(ii) inducing infiltration of CD8+ T cells to the brain metastasis.

According to some embodiments of the invention, the immunosuppressive immune cells comprise macrophages.

According to an aspect of some embodiments of the present invention there is provided an inhibitor of an MCP-1/CCR2/CCR4 axis for use in treating brain metastasis in a subject in need thereof.

According to an aspect of some embodiments of the present invention there is provided an article of manufacture comprising an inhibitor of an MCP-1/CCR2/CCR4 axis and an inhibitor of immune inhibitory molecule.

According to some embodiments of the invention, the brain metastasis is metastatic brain melanoma (MBM), metastatic breast cancer, metastatic gastrointestinal (GI) cancer and/or metastatic lung cancer.

According to some embodiments of the invention, the brain metastasis is metastatic brain melanoma (MBM) metastatic brain lung cancer or metastatic brain breast cancer.

According to some embodiments of the invention, the subject is post-surgery of the primary tumor for prevention and for treatment of surgically-removed brain metastases if resectable or for regression, if not resectable.

According to some embodiments of the invention, the administering is peripheral administration.

According to some embodiments of the invention, the administering is to the central nervous system (CNS).

According to some embodiments of the invention, the inhibitor of the MCP-1/CCR2 axis is selected from the group consisting of an antibody, a small molecule, a peptide, an aptamer, a genome editing agent, and a silencing nucleic acid which directly inhibits MCP-1 and/or CCR2.

According to some embodiments of the invention, the inhibitor inhibits MCP-1.

According to some embodiments of the invention, the inhibitor is selected from the group consisting of Carlumab, Bindarit and mNOX-E36.

According to some embodiments of the invention, the inhibitor inhibits CCR2.

According to some embodiments of the invention, the inhibitor is selected from the group consisting of S0916 (MLN1202), RS504393, PF-4136309 (INCB8761), BMS-813160, BMS-741672 and Cenicriviric (TAK-652, TBR-652).

According to some embodiments of the invention, the inhibitor inhibits CCR4.

According to some embodiments of the invention, the inhibitor is an anti MCP-1 antibody.

According to some embodiments of the invention, anti MCP-1 antibody is Carlumab.

According to some embodiments of the invention, the inhibitor is a small molecule.

According to some embodiments of the invention, the small molecule is Binadrit.

According to some embodiments of the invention, the inhibitor is a silencing nucleic acid.

According to some embodiments of the invention, the silencing nucleic acid is siRNA.

According to some embodiments of the invention, the siRNA is for silencing MCP-1, CCR2 or CCR4.

According to some embodiments of the invention, the method or use further comprising administering or using or including a modulator of an immune modulating molecule.

According to some embodiments of the invention, the immune inhibitory molecule comprises an immune checkpoint.

According to some embodiments of the invention, the modulating molecule is selected from the group consisting of PD-1, PD-L1, P-selectin, P-selectin Ligand-1, CTLA-4, OX-40, IDO, TIGIT, LAG-3 and ICOS.

According to some embodiments of the invention, the immune inhibitory molecule is a PD-1.

According to some embodiments of the invention, the inhibitor of an immune inhibitory molecule is an antibody.

According to some embodiments of the invention, the method or use further comprising an inhibitor of a cytokine selected from the group consisting of CCL5, GRO-alpha, PAI-1, IL-6 and IL-8/MIP-2.

According to some embodiments of the invention, the method or use further comprising administering or using or including an inhibitor of MEK and/or BRAF pathway.

According to some embodiments of the invention, the method or use further comprising further comprising determining an amount of selectin, P-selectin ligand-1, PD and/or PD-L1 in a biological sample of the subject, wherein presence or level of selectin, P-selectin ligand-1, PD and/or PD-L1 above a predetermined threshold is an indication for treatment with an inhibitor of an immune inhibitory molecule and optionally an inhibitor of selectin, P-selectin ligand-1, PD and/or PD-L1.

According to some embodiments of the invention, the method or use further comprising determining an amount of CCR2 in a biological sample of the subject, wherein presence or level of CCR2 above a predetermined threshold is an indication for treatment with an inhibitor of an immune inhibitory molecule.

According to some embodiments of the invention, the biological sample is selected from the group consisting of a tissue biopsy, a CSF sample, a blood same and a cellular or acellular fraction of a blood sample.

According to some embodiments of the invention, the metastatic brain melanoma is at an early stage comprising micro-metastases as opposed to macro-metastases.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWING(S)

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-D show that MCP-1 is a major astrocytes (AS)-secreted factor that influences melanoma migration. (A) Co-culture with AS and melanoma 3D multicellular spheroid (WM115 and D4M.3A mCherry-labeled melanoma cells). Mouse AS unlabeled. Human AS GFP-labeled in green. Dots represent melanoma cell invasion as fluorescence signal of mean±S.D. of n=3 spheroids/well of n=3 wells of three independent experiments (representative image of one independent experiment). Nonparametric Student's two-sided t-test statistical analysis. Scale bar—400 μm. (B) AS conditioned-media (CM—black) enhance melanoma Transwell® migration (WM115 and D4M.3A cells). Pixel density (total area covered per field) of the migrated cancer cells. Representative cell migrating fields are shown (n=3). Scale bar13 400 μm. Mean±S.D. of n=3 fields/well of three wells of three independent experiments. Nonparametric Student's t-test statistical analysis. (C) Levels of six pro-inflammatory AS-secreted cytokines in CM of melanoma cells (B16-F10—in black, A375—gray, 131/4-5B1—blue, WM115—red, and D4M.3A—green) or alternatively in SFM of melanoma cells for 24 h. Fold change ±S.D. of three independent biological repeats. (D) Melanoma cells (D4M.3A and WM115) were allowed to migrate in the presence of SFM, AS CM in the presence or absence of neutralizing antibodies. AS CM without LPS and AS CM was used as negative control for cell migration in D4M.3A and WM115, respectively. n=3, mean±S.D. of replicates of three independent biological repeats. One-way ANOVA statistical analysis.

FIGS. 2A-F show that inhibition of AS-secreted MCP-1 decreased migration. (A) MCP-1 mRNA in murine AS co-cultured with melanoma cells (B16-F10-black, B2905-gray, D4M.3A-dark green, Mel-ret-green) or human AS co-cultured in SFM with melanoma cells (WM115-red, 131/4-5B1-blue). Mean±S.D. of three independent biological repeats. Ordinary one-way ANOVA statistical analysis. (B) Wound healing migration assay of WM115 cells grown in AS CM (untreated—black empty dot), or siRNA:PEI targeting MCP-1 (blue), or NC (black full dot). Mean±S.D. of n=2 field per well biological replicates (n=3). Two-way ANOVA statistical analysis. (C) B16-F10—in black, D4M.3A—in green, WM115—in blue, A375—in gray exposed to bindarit for 72 h. Tumor cell viability assessed by metabolic activity MTT assay. Mean±S.D. of triplicates of three independent biological repeats. Nonparametric Student's t-test statistical analysis. (D) Secretion of MCP-1 in the CM of untreated, bindarit-treated astrocytes, melanoma CM-treated astrocytes, or melanoma CM-treated astrocytes treated with bindarit. Basal secretion of MCP-1 in WM115 melanoma cells (gray). Human astrocytes CM of astrocytes (hAS CM) (black); bindarit-treated astrocytes CM (red). Not-activated astrocytes (black), LPS-activated (light gray), melanoma CM-treated astrocytes (gray), bindarit-treated astrocytes (red). Mean±S.D. of triplicates of three independent biological repeats. One-way ANOVA statistical analysis. (E) Western blot of p-p65 in bindarit-treated astrocytes. Density bands of one representative experiment. p-p65 signal is presented as fold change expression relative to p65 expression. Density bands of p-p65 and p65 were normalized by vinculin (loading control). Mean±S.D. of triplicates of three independent biological repeats. (F) Transwell® migration of melanoma WM115 cells towards untreated AS (black), bindarit-treated AS (red), or bindarit-treated AS supplemented with rh-MCP-1 (blue). Representative fields of WM115 migrated cells (n=3). Scale bar—400 μm. Mean±S.D. of biological replicates (n=3). One-way ANOVA statistical analysis.

FIGS. 3A-E show that MCP-1 inhibition delays B16-F10 MBM progression. (A) B16-F10 melanoma cells were intracranially (i.cr.) inoculated in immunocompetent C57BL/6 mice to generate brain metastasis (n=20). Three days post tumor cell inoculation into the brain, mice were treated with 100 mg/kg i.v. bindarit (n=10) or PBS (n=10) QOD until day 15. Image created with BioRender.com. (B) Tumor size in MRI scans at day 13. Representative images of mice bearing brain tumors in PBS or bindarit-treated groups (n=10); H&E staining for tumor morphology and size. Scale bar—400 μm. Quantification of tumor size in B16-F10 brain tumor. Mean±S.D. (n=3 PBS; n=5 bindarit). Non-parametric Student's t-test statistical analysis. (C) Kaplan-Meier survival curve (n=7). Two-tailed P values from log-rank (Mantel-Cox). (D) Mice body weight change was monitored twice a week upon injection of B16-F10 melanoma cells. Mean±S.E.M. of n=10 per group. (E) Brain cryosections at day 13 (n=3) were stained for MCP-1 (red), GFAP (activated astrocytes—green), Iba1 (activated microglia/macrophages—green), IL-6 (green), F4/80 (macrophages—green), CD31 (blood vasculature—green), PD1/PD-L1 (exhausted T cells/inhibitory molecule—red/green), and CD8⁺ (cytotoxic T cells—green) markers. Nuclei are shown by DAPI staining (blue). Scale bar—100 μm. Mean±S.E.M. of n=7-10 fields per marker in n=3 mice per group. Nonparametric Student's t-test statistical analysis.

FIGS. 4A-C show that Bindarit treatment improves CD8 T-cell infiltration and decreases immune co-inhibitory molecules. (A) Tumor size and quantification of D4M.3A MBM bearing mice in MRI scans at day 10 and 14. Representative images of mice bearing brain tumors in PBS and bindarit-treated group. Mean±S.E.M. of n=4 in bindarit-treated group, n=5 in PBS-treated group. Nonparametric Student's t-test statistical analysis. (B) Tumor size and quantification of B16-F10 or Mel-ret MBM bearing mice in MRI scans at day 8. Representative images of mice bearing brain tumors in PBS and bindarit-treated group. Mean±S.E.M. of n=5 in bindarit and PBS-treated group. (C) Histological analysis at day 8 (representative fields of n=5-7 fields per marker in n=3 mice per group) of MCP-1/GFAP (in red and in green, respectively), CD206 staining associated to F4/80 macrophages (in red and in green, respectively), infiltration of CD8⁺ T cells/Iba1 microglia/microphages (red/green), tumor proliferation (Ki67—in red) and blood vasculature (CD31—in green), PD-1/PD-L1 exhausted T cells/inhibitory molecules (red/green) in B16-F10 MBM. Nuclei are shown by DAPI staining (blue). Scale bar—1000 μm (H&E) and 100 μm (IF). Mean±S.E.M. Nonparametric Student's t-test statistical analysis.

FIGS. 5A-D show that Bindarit-treated MBM resulted in a discrete although significant prolongation of the overall mice survival. (A-B) (A) Representative MRI scans, H&E staining and (B) tumor size quantification of B16-F10 and B2905 MBM bearing mice in PBS and bindarit-treated group. B16-F10 MBM—mean±S.E.M. of n=7 in bindarit-treated group and n=8 in PBS-treated group. B2905 MBM—Mean±S.E.M. of n=7 in PBS and bindarit-treated groups. Nonparametric Student's t-test statistical analysis was performed. (C) Kaplan-Meier survival curve of B16-F10 MBM bearing mice showed that bindarit prolonged survival. B16-F10 MBM—n=8 in PBS-treated group, n=7 in bindarit. B2905 MBM—n=8 in PBS and bindarit-treated group. Two-tailed p values from log-rank (Mantel-Cox). (D) Body weight change was monitored twice a week upon B16-F10 or B2905 tumor resection. Mean±S.E.M. of n=8 in PBS-treated group, n=7 in bindarit in B16-F10 MBM, and Mean±S.E.M. of n=8 in PBS and bindarit-treated groups.

FIGS. 6A-D show that astrocytes' activation and MCP-1 secretion may trigger CCR2/CCR4 overexpression in MBM. (A) GFAP/MCP-1 immunostaining (green/red) in activated astrocytes within melanoma samples and in normal brains of human and mouse. FFPE patient-derived-2 (PD-2); Cryo-section of MBM of mouse models (intracardiacally injection of mCherry-labeled D4M.3A cells, or intracranially injection of mCherry-labeled B16-F10 cells). Representative fields of n=3 MBM per cell line. Nuclei are shown by DAPI staining (blue). Scale bar—100 μm. Mean±S.D. (n=3-7 fields of n=3 MBM per cell line). Scale bar—100 μm. One-way ANOVA statistical analysis. (B) Representative fields of MCP-1/CCR2 and MCP-1/CCR4 staining in PD-2 and in mouse models of PM and MBM. Scale bar—100 μm. Nuclei are shown by DAPI staining (blue). Mean±S.D. n=7-10 fields of n=3 MBM per cell line. One-way ANOVA statistical analysis. (C) Quantification of (A) GFAP/MCP-1 immunostaining (green/red) in activated astrocytes within melanoma tumors and in to normal brain of human and mouse, presented in (D) Quantification of (B) MCP-1/CCR2 and MCP-1/CCR4 staining in PD-2 and in mouse models of PM and MBM.

FIGS. 7A-B show that CCR2/CCR4 CRISPR/Cas9 K/O recapitulated tumor growth and tumor landscape as observed following pharmacological inhibition of MCP-1. (A) Tumor size and quantification of K/O WT and NTC, shown by its relative tumor volume quantification of MRI scans and H&E staining. Mean±S.E.M. of n=10 mice per group. Ordinary one-way ANOVA statistical analysis. (B) Immune cell infiltration into tumors (A) was analyzed by FACS. For each marker analyzed the % of positive cells gated is presented. n=2 mice per group.

FIGS. 8A-J show that astrocytes and breast cancer cells have reciprocal interactions, enhancing proliferation, migration and invasion. (A) Proliferation rates of iRFP-labeled murine 4T1 cells, in the presence or absence of murine astrocytes (N=3) after 96 h. (B) Proliferation rates of iRFP-labeled human MDA-MB-231 cells, in the presence or absence of human astrocytes (N=3) after 88 h. Data represented as fold change to time 0; Statistical significance was determined using Friedman test (non-parametric one-way ANOVA) and Dunn's multiple comparisons test. (C) Quantification of transwell migration after 20 hours of MDA-MB-231 cells (N=4). Data represented as % of control (AM 0%). (D) Representative images of migrated cells from c. (E-F) Proliferation rates after 190 h, of (E) murine astrocytes in the presence or absence of 4T1 conditioned medium (N=4), and f(F) human astrocytes in the presence or absence of MDA-MB-231 conditioned medium (N=3). Data represented as fold change to time 0. (G-H) Quantification of tumor spheroids area after 48 h of (G) mCherry-labeled 4T1 cells, in the presence or absence of murine astrocytes (N=5), and (H) mCherry-labeled MDA-MB-231 cells, in the presence or absence of human astrocytes (N=4). Data represented as fold change to time 0. (I-J) Representative images of tumor spheroids from G-H (respectively). Scale bars=400 μm. All data are expressed as mean±SD; unless otherwise stated, statistical significance was determined using an unpaired Student t-test.

FIGS. 9A-D Show that astrocytes and breast cancer cells express pro-inflammatory cytokines, identifying MCP-1 as a major secreted factor in the settings of breast cancer-astrocyte co-culture. (A-B) Cytokine arrays of media collected following 72 h of cell culturing, mono-cultures and co-cultures of (A) murine 4T1 cells and murine astrocytes (N=1), and (B) human MDA-MB-231 cells and human astrocytes (N=1). (C-D) Quantification of signal intensity of the upregulated cytokines from A-B (respectively). Membrane negative controls are subtracted; data are normalized to membrane reference spots and expressed per million cells.

FIGS. 10A-F show that MCP-1-CCR2/4 axis is upregulated in brain metastases from breast cancer. (A) MCP-1 (red), Hoechst (blue) staining in human tissue sections of Brain Metastases from Breast Cancer, primary Breast Cancer and normal brains. Scale bars=100 μm. Representative images of selected fields. (B-C) MCP-1 secretion measured by ELISA of media collected after 72 h of cell culturing, from mono-cultures and co-cultures of (B) murine 4T1 cells and murine astrocytes (N=3), and (C) human MDA-MB-231 cells and human astrocytes (N=3). Data expressed as % of control; Statistical significance was determined using one-way ANOVA and Holm-Sidak's (B) or Tukey's (C) multiple comparisons tests. d, MCP-1 RNA expression measured by reverse transcription qPCR of RNA extracted from mCherry-labeled MDA-MB-231 cells and iRFP-labeled human astrocytes, after sorting the cells by their fluorescent labeling from either mono- or co-cultures (one representative out of N=5). Data expressed as fold change compared to human astrocytes from mono-culture; Statistical significance was determined using Two-Way ANOVA and Tukey's multiple comparisons test. (E-F) FACS analysis of 4T1 cells in murine astrocyte medium of (E) CCR4 expression, and (F) CCR2 expression. All data are expressed as mean±SD.

FIGS. 11A-G show that MCP-1-CCR2/4 axis is important for breast cancer proliferation, migration and invasion. (A) Proliferation rates after 72 h of mCherry-labeled 4T1 cells in the presence or absence of murine astrocytes and MCP-1 neutralizing antibody (N=1). Data represented as fold change to time 0; Statistical significance was determined using an unpaired Student t-test. (B) Proliferation rates mCherry-labeled MDA-MB-231 cells in the presence or absence of human astrocytes, MCP-1 neutralizing antibody (N=1). Data represented as fold change to time 0; Statistical significance was determined using one-way ANOVA and Sidak's multiple comparisons tests. (C-D) Proliferation rates of (C) iRFP-labeled 4T1 shCCR4 (or negative control sequence) cells in the presence or absence of murine astrocytes (N=1) after 94 h, and (D) iRFP-labeled MDA-MB-231 shCCR4 (or negative control sequence) cells in the presence or absence of murine astrocytes after 72 h. Data represented as fold change to time 0; Statistical significance was determined using One-way ANOVA and Sidak's multiple comparisons test. (E-F) Tumor spheroids of GFP-labeled MDA-MB-231 cells, in the presence or absence of iRFP-labeled human astrocytes and MCP-1 neutralizing antibody. (E) Representative images of tumor spheroids after 40 h. Scale bars=400 μm. (F) Quantification of tumor spheroids area after 40 h (N=1). Data represented as fold change to time 0; Statistical significance was determined using One-way ANOVA and Tukey's multiple comparisons test. (G) Quantification of MDA-MB-231 transwell migration after 20 h (N=1). Data represented as % of control (AM 0%); Statistical significance was determined using One-way ANOVA and Sidak's multiple comparisons test. All data are expressed as mean±SD.

FIG. 12 shows that MCP-1-CCR2 axis is upregulated in lung cancer brain metastases. MCP-1 (red), CCR2 (green), Hoechst (blue) staining in human tissue sections of Brain Metastases from Lung Cancer and normal brains. Scale bars=100 μm. Representative images of selected fields.

FIGS. 13A-H show that MCP-1/CCR2-CCR4, P-selectin/P-selectin ligand-1, PD-PD-L1 axes are upregulated in human BCBM. A. CCR2/CCR4/P-selectin (SELP)/P-selectin ligand-1 (PSGL-1) (red), Hoechst (blue) staining in human tissue sections of brain metastases, primary breast cancer, and normal brain. Scale bars=100 μm. Representative images of selected fields. B-H. mRNA expression analysis of B. MCP-1, C. CCR2, D. CCR4, E. SELP, F. PSGL-1, G. PD-1, and H. PD-L1, on GSE12276 dataset from primary breast tumors that metastasized to the brain, compared to primary breast tumors that metastasized to other organs. Statistical significance was determined using one-tailed non-paired Student's t-test.

FIGS. 14A-D show that MCP-1-CCR2/4 axis is important for breast cancer proliferation, migration and invasion. A-B. Proliferation rates after 160 hours of A. mCherry-labeled 4T1 cells in the presence or absence of murine astrocytes and MCP-1 inhibitor bindarit (one representative out of N=2), and B. mCherry-labeled MDA-MB-231 cells in the presence or absence of human astrocytes and MCP-1 inhibitor bindarit (N=3). Data represented as fold change to time 0. C. Quantification of MDA-MB-231 transwell migration after 20 hours (N=3). Data represented as % of control (Astrocyte Medium 0%). Representative images of migrated cells are presented below the graph. D. Tumor spheroid growth after 227 hours of mCherry-labeled MDA-MB-231 cells in the presence or absence of brain tumor microenvironment cells and different treatments. Data represented as fold change to time 0. Statistical significance was determined using One-way ANOVA and Tukey's multiple comparisons test. mAstro—murine astrocytes; hAstro—human astrocytes; HMG—human microglia; PBMCs—peripheral blood mononuclear cells; CM—conditioned medium; SELPi—small molecule P-selectin inhibitor; PD-L1i—small molecule programmed death-ligand 1 inhibitor.

FIG. 15 is a summary of the proposed interaction mechanism of MCP-1/CCR2 in melanoma brain metastasis. Primary melanoma (in the skin) locally invades the tissue, intravasates into the vasculature, and spreads to distant organs, especially to the brain. In this scenario, cancer cells, BME cells and in particular activated-astrocytes, secrete MCP-1 which in return, melanoma cells express CCR2. Tumor-associated macrophages/microglia, activated towards anti-inflammatory/pro-tumorigenic phenotypes together with regulatory T cells (Treg), are recruited to the tumor site. CD8+ T cells are poorly activated (CD107), and express PD-1 which results in uncontrolled tumor dissemination. Created with BioRender.com.

FIGS. 16A-G show that a combination therapy of bindarit with either P-selectin inhibitor or αPD-1 antibody, leads to favorable outcomes in orthotopic breast cancer brain metastases model. A. Timeline (days) of primary tumor inoculation, tumor resection, metastases induction (intracranial injection), treatments, and follow-up. B-C. Kaplan-Meier survival curves of the different treatment groups. Statistical significance was determined using survival analysis of each group compared to the control group, P values were further adjusted using Holm-Sidik analysis. D-E. Primary tumor recurrence and extracranial metastases development inhibition presented as the kinetics of development over time inside the groups. F. Quantification of brain metastases size 11 days post intracranial injection. Statistical significance was determined using two-tailed Student's t-test. G. Body weight change. All data are expressed as mean±SEM. SELPi—P-selectin inhibitor; PD-L1i—programmed death-ligand 1 inhibitor.

FIGS. 17A-C shows that a combination therapy of bindarit and nanoparticles containing BRAF\MEK inhibitors, leads to reduction in melanoma brain metastases 3D spheroid invasion. 3D Matrigel® invasion of melanoma (A) 131/4-5B1 mCherry cells or (B-C) D4M.3A mCherry cells grown in co-cultured with human or murine astrocytes (hAstrocytes (A), or mAstrocytes (B-C unlabeled), human or murine endothelial cells (hBECs in green (A), or mBECs unlabeled (B-C)) and unlabeled microglia (B-C). Treatments: (A-B) Untreated control (in black), 3 μM polyglutamic acid-Cy5 (PGA) (in grey), 3 μM SLM-DBF-diol (BRAF and MEK inhibitors (BRAFi and MEKi) (in yellow), PGA-Cy5 3 μM SLM-DBF (in red), 500 μM bindarit (in blue), 3 μM SLM-DBF-diol+500 μM bindarit (in green), PGA-Cy5 3 μM SLM-DBF+500 μM bindarit (in purple). (C) Untreated control (in black),1.75 μM poly (lactic-co-glycolic acid, PLGA) NP (empty NP) (in grey), 1.75 μM DBF (inhibitors (BRAFi) (in yellow), 1.75 μM DBF-NP (in red), 500 PM bindarit (in blue), 1.75 μM DBF-NP+500 μM bindarit (in green), 1.75 μM DBF NP+500 μM bindarit (in purple). Scale bar—400 μm.

FIGS. 18A-I show that a combination therapy of bindarit and nanovaccine, leads to favorable outcomes in orthotopic melanoma brain metastases model. (A) Schematic representation of prevention/intervention in vivo study. Briefly, B16-F10 melanoma cells were inoculated subcutaneously (100.000 cells), the tumors were resected when the average size reached 100-150 mm³. Ten days post resection of the primary tumor, mice were randomized and injected i.v. with one dose of bindarit (3 mg/kg free drug) every other day (QOD) until day 16 and intranasal administration of nano-vaccine containing mart-1 peptides (4.5 mg/kg) ones a week for three weeks. A week after primary tumor resection, B16-F10 melanoma cells (15.000 cells) were injected intracranially and mice were continuous treated (mice were injected i.v. with one dose of bindarit (3 mg/kg free drug) every other day (QOD) and intranasal administration of nano-vaccine containing mart-1 peptides (4.5 mg/kg) ones a week). (B) Representative images of MRI scans at day 14 and 18 post B16-F10 intracranially-injected cells. (C) MRI quantification of tumor growth at day 14 and 18 post B16-F10 intracranially-injected cells. In the group treated with the combination of nano-vaccine and bindarit, the tumor size was significantly reduced compared to PBS or mono-treatment groups. (D-H) Infiltration of immune cells into B16-F10 brain tumors was assessed followed tumor resection at day 18. The combination of nano-vaccine and bindarit (in blue) showed higher infiltration of (D) cytotoxic T cells, (F) antigen presenting cells (APC), (G) tissue-resident memory T cells (TRM), (H) effector T cells, and lower infiltration of regulatory T cells (Tregs) compared to PBS or the mono-treatment groups. (I). MART-1 positive cell cycle arrest was assessed followed isolation of melanoma cells from the brain at day 18. The combination of nano-vaccine and bindarit shows to induce cell cycle arrest at phase GO/1 (in blue).

FIGS. 19A-C shows that a combination therapy of bindarit and immune checkpoint inhibitors, following nanovaccine immunization, leads to reduced melanoma 3D spheroids invasion. (A) Schematic representation of prevention/intervention in vivo study. Briefly, B16-F10 melanoma cells were inoculated subcutaneously (100.000 cells), the tumor was resected when the average size reached 100-150 mm³. Ten days post resection of the primary tumor, mice were randomized and injected i.v. with one dose of bindarit (3 mg/kg free drug) every other day and intranasal administration of nano-vaccine containing mart-1 peptides (4.5 mg/kg), or PBS, once a week for three weeks. A week after primary tumor resection, B16-F10 melanoma cells (15,000 cells) were injected intracranially and mice were continuously treated as previously described. Following the last immunization with nano-vaccine, splenocytes were harvested, isolated, and activated with α-CD28 and β-mercaptoethanol (plain activation) or α-CD28 and β-mercapto and MHCI/II MART-1 peptides (nano-vaccine activation) for a week. Then, 3D spheroids of melanoma B16-F10 cells and astrocytes were co-cultured with splenocytes (plain activation or nano-vaccine activation) and treated with immuno-checkpoint inhibitors (anti-PD1 or anti-OX40) alone or in combination with bindarit (*treatment). (B-C) 3D Matrigel® invasion of melanoma (B) B16-F10 mCherry cells and astrocytes were co-cultured with a splenocytes (nano-vaccine) or (C) B16-F10 mCherry cells and astrocytes were co-cultured with splenocytes (plain activation). mCherry-labeled cell sprouting (in red) % of the total area covered per one spheroid. Nonparametric Student's t-test statistical analysis.

### DESCRIPTION OF SPECIFIC EMBODIMENTS OF THE INVENTION

The present invention, in some embodiments thereof, relates to treatment of brain metastasis.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Development of resistance to chemo- and immuno-therapies often occurs following treatment of brain metastasis such as in melanoma brain metastasis (MBM). In such cases astrocytes support brain metastasis progression by upregulating secreted-factors, amongst which the present inventors found that monocyte chemoattractant protein-1 (MCP-1) and its receptors, CCR2 and CCR4, are overexpressed in activated astrocytes and in brain metastatic melanoma cells compared to primary lesions. They show that melanoma cells alter astrocytes-secretome and evoke MCP-1 expression and secretion, which in turn enhance vascular hyperpermeability and proliferation, migration, and invasion of CCR2-expressing melanoma cells. Inhibition of MCP-1 rescues this phenotype. Pharmacological or molecular inhibition of MCP-1/CCR2 in MBM mouse model activates an anti-tumor immune-mediated response as revealed by the enhanced infiltration of cytotoxic CD8⁺ T cells, attenuated immunosuppressive phenotype of tumor-associated macrophages, and reduced infiltration of regulatory T cells, leading to inhibition of MBM progression and prolonged survival. Similar results were obtained with brain metastases from breast cancer or lung cancer. These results show that the MCP-1/CCR2 axis polarizes the brain microenvironment towards an anti-inflammatory/pro-tumorigenic phenotype, highlighting the therapeutic relevance of this pathway as a potential immune checkpoint in brain metastasis. Indeed the present inventors have shown that combining inhibition of MCP-1/CCR2/CCR4 inhibition with an inhibitor of immune inhibitory molecule acts in a favorable manner to alleviate brain metastasis.

Thus, according to an aspect of the invention there is provided a method of treating brain metastasis in a subject in need thereof, the method comprising administering to the subject a therapeutically effective amount of an inhibitor of an MCP-1/CCR2/CCR4 axis, thereby treating the brain metastasis in the subject.

According to an alternative or an additional aspect there is provided a method of activating an immune response against brain metastasis in a subject in need thereof, the method comprising administering to the subject a therapeutically effective amount of an inhibitor of an MCP-1/CCR2/CCR4 axis, thereby activating the immune response against brain metastasis in the subject.

According to a specific embodiment, activating an immune response is by at least one of:
(i) inducing polarization or activation of immunosuppressive immune cells (e.g., macrophages) from a pro-tumorogenic anti-inflammatory phenotype towards an immune-potentiator pro-inflammatory anti-tumorogenic phenotype; and
(ii) inducing infiltration of CD8+ T cells the said brain metastasis.

According to a specific embodiment, the immunosuppressive immune cells comprise macrophages and polarization is from an M2 phenotype to an M1 phenotype, as shown by the reduced expression of CCR2 and CD206 markers in F4/80 positive macrophages population following bindarit treatment as opposed to control (PBS-treated mice) group.

According to an alternative or an additional aspect there is provided an inhibitor of an MCP-1/CCR2/CCR4 axis for use in treating brain metastasis in a subject in need thereof.

According to an alternative or an additional aspect there is provided an article of manufacture comprising an inhibitor of an MCP-1/CCR2/CCR4 axis and an inhibitor of immune inhibitory molecule.

As used herein, the term “subject” refers to a mammal, such as a rodent, a feline, a canine, and a primate. Preferably, a subject according to the invention is a human.

As used herein, the term “treating” includes abrogating, substantially inhibiting, slowing or reversing the progression of a condition (i.e., brain metastasis), substantially ameliorating clinical or aesthetical symptoms of a condition or substantially preventing the appearance of clinical or aesthetical symptoms of a condition.

Treatment may be augmented using Gold-standard methods such as surgery, radiation therapy, chemotherapy, or a combination of treatments.

As used herein, the term “preventing” refers to keeping a disease, disorder or condition (i.e., brain metastasis) from occurring in a subject who may be at risk for the disease, but has not yet been diagnosed as having the disease.

As used herein “brain metastasis” refers to the occurrence of spread of cancer cells from an original side which is not the CNS (e.g., brain) to the CNS (e.g., brain).

According to a specific embodiment, the subject is diagnosed with brain metastasis.

According to other embodiments, the subject is diagnosed with cancer that may lead to brain metastasis.

Metastatic brain tumors include, but are not limited to, lung, breast, melanoma, colon, kidney and thyroid gland cancers.

According to a specific embodiment, the brain metastasis is metastatic brain melanoma (MBM), metastatic breast cancer, metastatic gastrointestinal (GI) cancer and/or metastatic lung cancer.

According to a specific embodiment, the brain metastasis is metastatic brain melanoma (MBM) metastatic brain lung cancer or metastatic brain breast cancer.

Brain metastases may form one tumor or many tumors in the brain. As the metastatic brain tumors grow, they create pressure on and change the function of surrounding brain tissue. This causes signs and symptoms, such as headache, personality changes, memory loss and seizures.

In one embodiment, the subject is a non-operable and/or non-irradiable subject. In one embodiment, the subject has a tumor comprising two or more lobes. According to other embodiments, the brain metastasis is operable.

According to a specific embodiment, the subject is post-surgery of the primary tumor for prevention of occurrence of brain metastasis.

According to a specific embodiment, the subject is post surgically-removed brain metastases.

According to other embodiments the subject is pre-removal of the brain metastasis if resectable.

According to some embodiments, the metastatic brain melanoma is at an early stage comprising micro-metastases as opposed to macro-metastases.

The subject may be selected for treatment by determining an amount of CCR2 in a biological sample of the subject, wherein presence or level of CCR2 above a predetermined threshold is an indication for treatment with an inhibitor of an immune inhibitory molecule.

Alternatively or additionally, the selection is performed by determining an amount of selectin, P-selectin ligand-1, PD and/or PD-L1 in a biological sample of the subject, wherein presence or level of selectin, P-selectin ligand-1, PD and/or PD-L1 above a predetermined threshold is an indication for treatment with an inhibitor of an immune inhibitory molecule and optionally an inhibitor of selectin, P-selectin ligand-1, PD and/or PD-L1.

According to some embodiments, the threshold is determined as compared to the average level of the same target of detection (e.g., CCR2) in the same type of biological sample of healthy subjects. However, the exact method for determining a cut-off will be determined by the skilled artisan.

As used herein “amount” refers to mRNA or protein. Methods of determining mRNA or protein in a biological sample are known in the art, a description of some is provided below.

According to some embodiments, the biological sample is selected from the group consisting of a tissue biopsy, a CSF sample, a blood sample and a cellular or acellular fraction of a blood sample.

As used herein “MCP-1/CCR2/CCR4 axis” or “MCP-1/CCR2/CCR4 system” refers to the binding of the soluble molecule MCP-1 to its cognate receptors CCR2 and CCR4 which leads to activation of astrocytes and migration of tumor cells.

As used herein “monocyte chemoattractant protein 1 (MCP1)” which is also referred to herein as “chemokine (C-C motif) ligand 2 (CCL2)” or “small inducible cytokine A2” is a small cytokine that belongs to the CC chemokine family. It is encoded by the CCL2 gene and in human has the protein sequence NP_002973 and the mRNA sequence NM_002982. MCP1 tightly regulates cellular mechanics^{[5]} and thereby recruits monocytes, memory T cells, and dendritic cells to the sites of inflammation produced by either tissue injury or infection.

As used herein “CCR2” refers to C-C chemokine receptor type 2 (CCR2 or CD192 (cluster of differentiation 192). It is a protein that in humans is encoded by the CCR2 gene. CCR2 is a CC chemokine receptor. Two alternatively spliced transcript variants are expressed by the gene: protein accessions in human: NP_001116513 and NP_001116868.1 and mRNA accessions: NM_001123041 and NM_001123396.

As used herein “CCR4” refers to C-C chemokine receptor type 4 is a protein that in humans is encoded by the CCR4 gene. CCR4 has also recently been designated CD194 (cluster of differentiation 194). It binds several chemokines one if which if CCL2 (MCP1). The protein product in human has the accession NP_005499 and the mRNA product has the accession NM_005508.

As used herein “an inhibitor” refers to a molecule that down-regulates an activity or expression (amount) of the mRNA or protein product of a target gene. According to a specific embodiment, the inhibitor down-regulates the activity or expression of any of MCP-1, CCR2, CCR4.

According to a specific embodiment, the inhibitor inhibits the activity or expression of the mRNA or protein product of a target gene in a direct manner.

As used herein “direct manner” or “directly” refers to the mode of action of the inhibitor. That is for example, by direct interaction with the target of inhibition, e.g., MCP-1 or CCR2 or CCR4 or by eliciting a function directly on MCP-1, CCR2 or CCR4 and not exclusively an effector molecule of this axis or not exclusively on another CCR receptor or CCL ligand.

Alternatively, the inhibitor inhibits the MCP-1/CCR2/CCR4 in an indirect manner such as on an effector of this pathway (e.g., PI3K pathway or JAK pathway, see e.g., Maosen et al., 2021 www(dot)doi(dot)org/10.1111/cpr(dot)13115).

According to a specific embodiment, the inhibitor is a direct inhibitor i.e., binds/interacts with the target of inhibition, i.e., MCP-1, CCR2 or CCR4.

According to a specific embodiment, the inhibitor is an indirect inhibitor i.e., binds/interacts with an activator of effects of the target of inhibition, i.e., MCP-1, CCR2 or CCR4.

As used herein the phrase “downregulates expression or activity” refers to downregulating the expression of a target gene at the genomic (e.g. homologous recombination and site specific endonucleases) and/or the transcript level using a variety of molecules which interfere with transcription and/or translation (e.g., RNA silencing agents, CRISPR/Cas-9), resulting in less amount of the protein in the cell or secreted therefrom; or down regulation at the protein level (e.g., small molecules, inhibitory peptides, enzymes that cleave the polypeptide, antibodies, aptamers and the like), which can either result in less protein product or reduced activity thereof all compared to same in a cell of an identical type, age and growth conditions, which has not been treated with the inhibitor.

Thus, for example, for the same culture conditions, the expression is generally expressed in comparison to the expression in a cell of the same species but not contacted with the agent or contacted with a vehicle control, also referred to as control.

Down regulation of expression may be either transient or permanent.

According to specific embodiments, down regulating expression refers to the absence of mRNA and/or protein, as detected by RT-PCR or Western blot, respectively.

According to other specific embodiments down regulating expression refers to a decrease in the level of mRNA and/or protein, as detected by RT-PCR or Western blot, respectively. The reduction may be by at least a 10%, at least 20%, at least 30%, at least 40%, at least 50%, at least 60%, at least 70%, at least 80%, at least 90%, at least 95% or at least 99% reduction.

Non-limiting examples of agents capable of down regulating expression are described in details hereinbelow.

Down-regulation at the nucleic acid level is typically effected using a nucleic acid agent, having a nucleic acid backbone, DNA, RNA, mimetics thereof or a combination of same. The nucleic acid agent may be encoded from a DNA molecule or provided to the cell per se.

According to specific embodiments, the downregulating agent is a polynucleotide.

According to specific embodiments, the downregulating agent is a polynucleotide capable of hybridizing to a target gene or mRNA encoding same.

According to specific embodiments, the downregulating agent is a polynucleotide capable of hybridizing to a gene or mRNA encoding MCP1, CCR2 or CCR4 (other targets are described herein in the document, e.g., PD1, P-Selectin and others).

According to specific embodiments the downregulating agent is an RNA silencing agent or a genome editing agent.

Thus, downregulation of MCP1, CCR2 or CCR4 (other targets are described herein in the document, e.g., PD1, P-Selectin and others) can be achieved by RNA silencing.

As used herein, the phrase “RNA silencing” refers to a group of regulatory mechanisms [e.g. RNA interference (RNAi), transcriptional gene silencing (TGS), post-transcriptional gene silencing (PTGS), quelling, co-suppression, and translational repression] mediated by RNA molecules which result in the inhibition or “silencing” of the expression of a corresponding protein-coding gene. RNA silencing has been observed in many types of organisms, including plants, animals, and fungi.

As used herein, the term “RNA silencing agent” refers to an RNA which is capable of specifically inhibiting or “silencing” the expression of a target gene. In certain embodiments, the RNA silencing agent is capable of preventing complete processing (e.g., the full translation and/or expression) of an mRNA molecule through a post-transcriptional silencing mechanism. RNA silencing agents include non-coding RNA molecules, for example RNA duplexes comprising paired strands, as well as precursor RNAs from which such small non-coding RNAs can be generated. Exemplary RNA silencing agents include dsRNAs such as siRNAs, miRNAs and shRNAs. The RNA silencing agent may be administered as a naked oligonucleotide, may comprise modified bases, may be a stabilized oligonucleotide, entrapped or conjugated to a nanoparticle or any nanocarrier (polymers, lipids-LNP, liposomes, micelles, etc.)—see for example Scomparin et al Biotechnology Advances, Volume 33, Issue 6, Part 3, 1 Nov. 2015, Pages 1294-1309. In one embodiment, the RNA silencing agent is capable of inducing RNA interference.

In another embodiment, the RNA silencing agent is capable of mediating translational repression.

According to an embodiment of the invention, the RNA silencing agent is specific to the target RNA (e.g.,) and does not cross inhibit or silence other targets or a splice variant which exhibits 99% or less global homology to the target gene, e.g., less than 98%, 97%, 96%, 95%, 94%, 93%, 92%, 91%, 90%, 89%, 88%, 87%, 86%, 85%, 84%, 83%, 82%, 81% global homology to the target gene; as determined by PCR, Western blot, Immunohistochemistry and/or flow cytometry.

RNA interference refers to the process of sequence-specific post-transcriptional gene silencing in animals mediated by short interfering RNAs (siRNAs).

Following is a detailed description on RNA silencing agents that can be used according to specific embodiments of the present invention.

DsRNA, siRNA and shRNA—The presence of long dsRNAs in cells stimulates the activity of a ribonuclease III enzyme referred to as dicer. Dicer is involved in the processing of the dsRNA into short pieces of dsRNA known as short interfering RNAs (siRNAs). Short interfering RNAs derived from dicer activity are typically about 21 to about 23 nucleotides in length and comprise about 19 base pair duplexes. The RNAi response also features an endonuclease complex, commonly referred to as an RNA-induced silencing complex (RISC), which mediates cleavage of single-stranded RNA having sequence complementary to the antisense strand of the siRNA duplex. Cleavage of the target RNA takes place in the middle of the region complementary to the antisense strand of the siRNA duplex.

Accordingly, some embodiments of the invention contemplate use of dsRNA to downregulate protein expression from mRNA.

According to one embodiment dsRNA longer than 30 bp are used. Various studies demonstrate that long dsRNAs can be used to silence gene expression without inducing the stress response or causing significant off-target effects—see for example [Strat et al., Nucleic Acids Research, 2006, Vol. 34, No. 13 3803-3810; Bhargava A et al. Brain Res. Protoc. 2004; 13:115-125; Diallo M., et al., Oligonucleotides. 2003; 13:381-392; Paddison P. J., et al., Proc. Natl Acad. Sci. USA. 2002; 99:1443-1448; Tran N., et al., FEBS Lett. 2004; 573:127-134].

According to some embodiments of the invention, dsRNA is provided in cells were the interferon pathway is not activated, see for example Billy et al., PNAS 2001, Vol 98, pages 14428-14433 and Diallo et al., Oligonucleotides, Oct. 1, 2003, 13(5): 381-392. doi:10.1089/154545703322617069.

According to an embodiment of the invention, the long dsRNA are specifically designed not to induce the interferon and PKR pathways for down-regulating gene expression. For example, Shinagwa and Ishii [Genes & Dev. 17 (11): 1340-1345, 2003] have developed a vector, named pDECAP, to express long double-strand RNA from an RNA polymerase II (Pol II) promoter. Because the transcripts from pDECAP lack both the 5′-cap structure and the 3′-poly(A) tail that facilitate ds-RNA export to the cytoplasm, long ds-RNA from pDECAP does not induce the interferon response.

Another method of evading the interferon and PKR pathways in mammalian systems is by introduction of small inhibitory RNAs (siRNAs) either via transfection or endogenous expression.

The term “siRNA” refers to small interfering RNA duplexes (generally between 18-30 base pairs) that induce the RNA interference (RNAi) pathway. Typically, siRNAs are chemically synthesized as 21mers with a central 19 bp duplex region and symmetric 2-base 3′-overhangs on the termini, although it has been recently described that chemically synthesized RNA duplexes of 25-30 base length can have as much as a 100-fold increase in potency compared with 21mers at the same location. The observed increased potency obtained using longer RNAs in triggering RNAi is suggested to result from providing Dicer with a substrate (27mer) instead of a product (21mer) and that this improves the rate or efficiency of entry of the siRNA duplex into RISC.

It has been found that position of the 3′-overhang influences potency of an siRNA and asymmetric duplexes having a 3′-overhang on the antisense strand are generally more potent than those with the 3′-overhang on the sense strand (Rose et al., 2005). This can be attributed to asymmetrical strand loading into RISC, as the opposite efficacy patterns are observed when targeting the antisense transcript.

The strands of a double-stranded interfering RNA (e.g., a siRNA) may be connected to form a hairpin or stem-loop structure (e.g., a shRNA). Thus, as mentioned, the RNA silencing agent of some embodiments of the invention may also be a short hairpin RNA (shRNA).

miRNA and miRNA mimics—According to another embodiment the RNA silencing agent may be a miRNA.

The term “microRNA”, “miRNA”, and “miR” are synonymous and refer to a collection of non-coding single-stranded RNA molecules of about 19-28 nucleotides in length, which regulate gene expression. miRNAs are found in a wide range of organisms and have been shown to play a role in development, homeostasis, and disease etiology.

Antisense—Antisense is a single stranded RNA designed to prevent or inhibit expression of a gene by specifically hybridizing to its mRNA. Downregulation of a can be effected using an antisense polynucleotide capable of specifically hybridizing with an mRNA transcript encoding MCP1, CCR2 or CCR4 (other targets are described herein in the document, e.g., PD1, P-Selectin and others). Downregulation of MCP1, CCR2 or CCR4 (other targets are described herein in the document, e.g., PD1, P-Selectin and others) can be effected using an antisense polynucleotide capable of specifically hybridizing with an mRNA transcript encoding MCP1, CCR2 or CCR4 (other targets are described herein in the document, e.g., PD1, P-Selectin and others).

Design of antisense molecules, which can be used to efficiently downregulate a MCP1, CCR2 or CCR4 (other targets are described herein in the document, e.g., PD1, P-Selectin and others) must be effected while considering two aspects important to the antisense approach. The first aspect is delivery of the oligonucleotide into the cytoplasm of the appropriate cells, while the second aspect is design of an oligonucleotide, which specifically binds the designated mRNA within cells in a way which inhibits translation thereof.

Downregulation can be achieved by inactivating the gene, i.e. the gene of MCP1, CCR2 or CCR4 (other targets are described herein in the document, e.g., PD1, P-Selectin and others), via introducing targeted mutations involving loss-of function alterations (e.g. point mutations, deletions and insertions) in the gene structure.

As used herein, the phrase “loss-of-function alterations” refers to any mutation in the DNA sequence of a gene which results in downregulation of the expression level and/or activity of the expressed product, i.e., the mRNA transcript and/or the translated protein. Non-limiting examples of such loss-of-function alterations include a missense mutation, i.e., a mutation which changes an amino acid residue in the protein with another amino acid residue and thereby abolishes the enzymatic activity of the protein; a nonsense mutation, i.e., a mutation which introduces a stop codon in a protein, e.g., an early stop codon which results in a shorter protein devoid of the enzymatic activity; a frame-shift mutation, i.e., a mutation, usually, deletion or insertion of nucleic acid(s) which changes the reading frame of the protein, and may result in an early termination by introducing a stop codon into a reading frame (e.g., a truncated protein, devoid of the enzymatic activity), or in a longer amino acid sequence (e.g., a readthrough protein) which affects the secondary or tertiary structure of the protein and results in a non-functional protein, devoid of the enzymatic activity of the non-mutated polypeptide; a readthrough mutation due to a frame-shift mutation or a modified stop codon mutation (i.e., when the stop codon is mutated into an amino acid codon), with an abolished enzymatic activity; a promoter mutation, i.e., a mutation in a promoter sequence, usually 5′ to the transcription start site of a gene, which results in down-regulation of a specific gene product; a regulatory mutation, i.e., a mutation in a region upstream or downstream, or within a gene, which affects the expression of the gene product; a deletion mutation, i.e., a mutation which deletes coding nucleic acids in a gene sequence and which may result in a frame-shift mutation or an in-frame mutation (within the coding sequence, deletion of one or more amino acid codons); an insertion mutation, i.e., a mutation which inserts coding or non-coding nucleic acids into a gene sequence, and which may result in a frame-shift mutation or an in-frame insertion of one or more amino acid codons; an inversion, i.e., a mutation which results in an inverted coding or non-coding sequence; a splice mutation i.e., a mutation which results in abnormal splicing or poor splicing; and a duplication mutation, i.e., a mutation which results in a duplicated coding or non-coding sequence, which can be in-frame or can cause a frame-shift.

According to specific embodiments loss-of-function alteration of a gene may comprise at least one allele of the gene.

The term “allele” as used herein, refers to any of one or more alternative forms of a gene locus, all of which alleles relate to a trait or characteristic. In a diploid cell or organism, the two alleles of a given gene occupy corresponding loci on a pair of homologous chromosomes.

According to other specific embodiments, loss-of-function alteration of a gene comprises both alleles of the gene. In such instances, the MCP1, CCR2 or CCR4 gene (other targets are described herein in the document, e.g., PD1, P-Selectin and others) may be in a homozygous form or in a heterozygous form.

Methods of introducing nucleic acid alterations to a gene of interest are well known in the art [see for example Menke D. Genesis (2013) 51:- 618; Capecchi, Science (1989) 244:1288-1292; Santiago et al. Proc Natl Acad Sci USA (2008) 105:5809-5814; International Patent Application Nos. WO 2014085593, WO 2009071334 and WO 2011146121; U.S. Pat. Nos. 8,771,945, 8,586,526, 6,774,279 and UP Patent Application Publication Nos. 20030232410, 20050026157, US20060014264; the contents of which are incorporated by reference in their entireties] and include targeted homologous recombination, site specific recombinases, PB transposases and genome editing by engineered nucleases. Agents for introducing nucleic acid alterations to a gene of interest can be designed publically available sources or obtained commercially from Transposagen, Addgene and Sangamo Biosciences.

Following is a description of various exemplary methods used to introduce nucleic acid alterations to a gene of interest and agents for implementing same that can be used according to specific embodiments of the present invention.

Genome Editing using engineered endonucleases—this approach refers to a reverse genetics method using artificially engineered nucleases to cut and create specific double-stranded breaks at a desired location(s) in the genome, which are then repaired by cellular endogenous processes such as, homology directed repair (HDR) and non-homologous end-joining (NHEJ). NHEJ directly joins the DNA ends in a double-stranded break, while HDR utilizes a homologous sequence as a template for regenerating the missing DNA sequence at the break point. In order to introduce specific nucleotide modifications to the genomic DNA, a DNA repair template containing the desired sequence must be present during HDR. Genome editing cannot be performed using traditional restriction endonucleases since most restriction enzymes recognize a few base pairs on the DNA as their target and the probability is very high that the recognized base pair combination will be found in many locations across the genome resulting in multiple cuts not limited to a desired location. To overcome this challenge and create site-specific single- or double-stranded breaks, several distinct classes of nucleases have been discovered and bioengineered to date. These include the meganucleases, Zinc finger nucleases (ZFNs), transcription-activator like effector nucleases (TALENs) and CRISPR/Cas system.

Meganucleases—Meganucleases are commonly grouped into four families: the LAGLIDADG family, the GIY-YIG family, the His-Cys box family and the HNH family. These families are characterized by structural motifs, which affect catalytic activity and recognition sequence. For instance, members of the LAGLIDADG family are characterized by having either one or two copies of the conserved LAGLIDADG motif. The four families of meganucleases are widely separated from one another with respect to conserved structural elements and, consequently, DNA recognition sequence specificity and catalytic activity. Meganucleases are found commonly in microbial species and have the unique property of having very long recognition sequences (>14 bp) thus making them naturally very specific for cutting at a desired location. This can be exploited to make site-specific double-stranded breaks in genome editing. One of skill in the art can use these naturally occurring meganucleases, however the number of such naturally occurring meganucleases is limited. To overcome this challenge, mutagenesis and high throughput screening methods have been used to create meganuclease variants that recognize unique sequences. For example, various meganucleases have been fused to create hybrid enzymes that recognize a new sequence. Alternatively, DNA interacting amino acids of the meganuclease can be altered to design sequence specific meganucleases (see e.g., U.S. Pat. No. 8,021,867). Meganucleases can be designed using the methods described in e.g., Certo, M T et al. Nature Methods (2012) 9:073-975; U.S. Pat. Nos. 8,304,222; 8,021,867; 8,119,381; 8,124,369; 8,129,134; 8,133,697; 8,143,015; 8,143,016; 8, 148,098; or 8, 163,514, the contents of each are incorporated herein by reference in their entirety. Alternatively, meganucleases with site-specific cutting characteristics can be obtained using commercially available technologies e.g., Precision Biosciences' Directed Nuclease Editor™ genome editing technology.

ZFNs and TALENs—Two distinct classes of engineered nucleases, zinc-finger nucleases (ZFNs) and transcription activator-like effector nucleases (TALENs), have both proven to be effective at producing targeted double-stranded breaks (Christian et al., 2010; Kim et al., 1996; Li et al., 2011; Mahfouz et al., 2011; Miller et al., 2010).

Basically, ZFNs and TALENs restriction endonuclease technology utilizes a non-specific DNA cutting enzyme which is linked to a specific DNA binding domain (either a series of zinc finger domains or TALE repeats, respectively). Typically a restriction enzyme whose DNA recognition site and cleaving site are separate from each other is selected. The cleaving portion is separated and then linked to a DNA binding domain, thereby yielding an endonuclease with very high specificity for a desired sequence. An exemplary restriction enzyme with such properties is Fok1. Additionally Fok1 has the advantage of requiring dimerization to have nuclease activity and this means the specificity increases dramatically as each nuclease partner recognizes a unique DNA sequence. To enhance this effect, Fok1 nucleases have been engineered that can only function as heterodimers and have increased catalytic activity. The heterodimer functioning nucleases avoid the possibility of unwanted homodimer activity and thus increase specificity of the double-stranded break.

Thus, for example to target a specific site, ZFNs and TALENs are constructed as nuclease pairs, with each member of the pair designed to bind adjacent sequences at the targeted site. Upon transient expression in cells, the nucleases bind to their target sites and the FokI domains heterodimerize to create a double-stranded break. Repair of these double-stranded breaks through the non-homologous end-joining (NHEJ) pathway most often results in small deletions or small sequence insertions. Since each repair made by NHEJ is unique, the use of a single nuclease pair can produce an allelic series with a range of different deletions at the target site. The deletions typically range anywhere from a few base pairs to a few hundred base pairs in length, but larger deletions have successfully been generated in cell culture by using two pairs of nucleases simultaneously (Carlson et al., 2012; Lee et al., 2010). In addition, when a fragment of DNA with homology to the targeted region is introduced in conjunction with the nuclease pair, the double-stranded break can be repaired via homology directed repair to generate specific modifications (Li et al., 2011; Miller et al., 2010; Urnov et al., 2005).

Although the nuclease portions of both ZFNs and TALENs have similar properties, the difference between these engineered nucleases is in their DNA recognition peptide. ZFNs rely on Cys2-His2 zinc fingers and TALENs on TALEs. Both of these DNA recognizing peptide domains have the characteristic that they are naturally found in combinations in their proteins. Cys2-His2 Zinc fingers typically found in repeats that are 3 bp apart and are found in diverse combinations in a variety of nucleic acid interacting proteins. TALEs on the other hand are found in repeats with a one-to-one recognition ratio between the amino acids and the recognized nucleotide pairs. Because both zinc fingers and TALEs happen in repeated patterns, different combinations can be tried to create a wide variety of sequence specificities. Approaches for making site-specific zinc finger endonucleases include, e.g., modular assembly (where Zinc fingers correlated with a triplet sequence are attached in a row to cover the required sequence), OPEN (low-stringency selection of peptide domains vs. triplet nucleotides followed by high-stringency selections of peptide combination vs. the final target in bacterial systems), and bacterial one-hybrid screening of zinc finger libraries, among others. ZFNs can also be designed and obtained commercially from e.g., Sangamo Biosciences™ (Richmond, CA).

Method for designing and obtaining TALENs are described in e.g. Reyon et al. Nature Biotechnology 2012 May; 30(5):460-5; Miller et al. Nat Biotechnol. (2011) 29: 143-148; Cermak et al. Nucleic Acids Research (2011) 39 (12): e82 and Zhang et al. Nature Biotechnology (2011) 29 (2): 149-53. A recently developed web-based program named Mojo Hand was introduced by Mayo Clinic for designing TAL and TALEN constructs for genome editing applications (can be accessed through www(dot)talendesign(dot)org). TALEN can also be designed and obtained commercially from e.g., Sangamo Biosciences™ (Richmond, CA).

CRISPR-Cas system—Many bacteria and archaea contain endogenous RNA-based adaptive immune systems that can degrade nucleic acids of invading phages and plasmids. These systems consist of clustered regularly interspaced short palindromic repeat (CRISPR) genes that produce RNA components and CRISPR associated (Cas) genes that encode protein components. The CRISPR RNAs (crRNAs) contain short stretches of homology to specific viruses and plasmids and act as guides to direct Cas nucleases to degrade the complementary nucleic acids of the corresponding pathogen. Studies of the type II CRISPR/Cas system of Streptococcus pyogenes have shown that three components form an RNA/protein complex and together are sufficient for sequence-specific nuclease activity: the Cas9 nuclease, a crRNA containing 20 base pairs of homology to the target sequence, and a trans-activating crRNA (tracrRNA) (Jinek et al. Science (2012) 337: 816-821.). It was further demonstrated that a synthetic chimeric guide RNA (gRNA) composed of a fusion between crRNA and tracrRNA could direct Cas9 to cleave DNA targets that are complementary to the crRNA in vitro. It was also demonstrated that transient expression of Cas9 in conjunction with synthetic gRNAs can be used to produce targeted double-stranded brakes in a variety of different species (Cho et al., 2013; Cong et al., 2013; DiCarlo et al., 2013; Hwang et al., 2013a,b; Jinek et al., 2013; Mali et al., 2013).

The CRIPSR/Cas system for genome editing contains two distinct components: a gRNA and an endonuclease e.g. Cas9.

The gRNA is typically a 20 nucleotide sequence encoding a combination of the target homologous sequence (crRNA) and the endogenous bacterial RNA that links the crRNA to the Cas9 nuclease (tracrRNA) in a single chimeric transcript. The gRNA/Cas9 complex is recruited to the target sequence by the base-pairing between the gRNA sequence and the complement genomic DNA. For successful binding of Cas9, the genomic target sequence must also contain the correct Protospacer Adjacent Motif (PAM) sequence immediately following the target sequence. The binding of the gRNA/Cas9 complex localizes the Cas9 to the genomic target sequence so that the Cas9 can cut both strands of the DNA causing a double-strand break. Just as with ZFNs and TALENs, the double-stranded brakes produced by CRISPR/Cas can undergo homologous recombination or NHEJ.

The Cas9 nuclease has two functional domains: RuvC and HNH, each cutting a different DNA strand. When both of these domains are active, the Cas9 causes double strand breaks in the genomic DNA.

A significant advantage of CRISPR/Cas is that the high efficiency of this system coupled with the ability to easily create synthetic gRNAs enables multiple genes to be targeted simultaneously. In addition, the majority of cells carrying the mutation present biallelic mutations in the targeted genes.

However, apparent flexibility in the base-pairing interactions between the gRNA sequence and the genomic DNA target sequence allows imperfect matches to the target sequence to be cut by Cas9.

Modified versions of the Cas9 enzyme containing a single inactive catalytic domain, either RuvC- or HNH-, are called ‘nickases’. With only one active nuclease domain, the Cas9 nickase cuts only one strand of the target DNA, creating a single-strand break or ‘nick’. A single-strand break, or nick, is normally quickly repaired through the HDR pathway, using the intact complementary DNA strand as the template. However, two proximal, opposite strand nicks introduced by a Cas9 nickase are treated as a double-strand break, in what is often referred to as a ‘double nick’ CRISPR system. A double-nick can be repaired by either NHEJ or HDR depending on the desired effect on the gene target. Thus, if specificity and reduced off-target effects are crucial, using the Cas9 nickase to create a double-nick by designing two gRNAs with target sequences in close proximity and on opposite strands of the genomic DNA would decrease off-target effect as either gRNA alone will result in nicks that will not change the genomic DNA.

Modified versions of the Cas9 enzyme containing two inactive catalytic domains (dead Cas9, or dCas9) have no nuclease activity while still able to bind to DNA based on gRNA specificity. The dCas9 can be utilized as a platform for DNA transcriptional regulators to activate or repress gene expression by fusing the inactive enzyme to known regulatory domains. For example, the binding of dCas9 alone to a target sequence in genomic DNA can interfere with gene transcription.

There are a number of publicly available tools available to help choose and/or design target sequences as well as lists of bioinformatically determined unique gRNAs for different genes in different species such as the Feng Zhang lab's Target Finder, the Michael Boutros lab's Target Finder (E-CRISP), the RGEN Tools: Cas-OFFinder, the CasFinder: Flexible algorithm for identifying specific Cas9 targets in genomes and the CRISPR Optimal Target Finder.

Non-limiting examples of gRNA sequences that can be used with some embodiments of the present invention are described in the literature (Sanjana N.E., Shalem O., Zhang F. Nat Methods. 2014 August; 11(8):783-4) and in the genscript website see www(dot)genscript(dot)com/gRNA-detail/6403/SELP-CRISPR-guide-RNA.

According to specific embodiments, the gRNA sequence does not have a significant off target effect. Methods of determining off target effect are well known in the art, such as BGI Human Whole Genome Sequencing (described in Nature; 491:65-56.2012), next generation sequencing (NGS) using e.g. commercially available kits such as Alt-R-Genom Editing (IDT detection kit) or Sure select target enrich <1% variant allele frequency (Agilent).

In order to use the CRISPR system, both gRNA and Cas9 should be expressed in a target cell. The insertion vector can contain both cassettes on a single plasmid or the cassettes are expressed from two separate plasmids. CRISPR plasmids are commercially available such as the px330 plasmid from Addgene. Alternatively, the target cell can be transfected with both gRNA and Cas9 without plasmid using e.g. a transfection reagent such as CRISPRMAX [see e.g. Yu et al. (2016) JD1Biotechnol Lett. 38(6):919-29]. In some cells electroporation can improve the transfection of the gRNA and the Cas9 [see e.g. Liang et al. (2015) Journal of Biotechnology 208, 2015, Pages 44-53; and Liang et al. (2017) Journal of Biotechnology, Volume 241, 2017, pp. 136-146].

“Hit and run” or “in-out”—involves a two-step recombination procedure. In the first step, an insertion-type vector containing a dual positive/negative selectable marker cassette is used to introduce the desired sequence alteration. The insertion vector contains a single continuous region of homology to the targeted locus and is modified to carry the mutation of interest. This targeting construct is linearized with a restriction enzyme at a one site within the region of homology, electroporated into the cells, and positive selection is performed to isolate homologous recombinants. These homologous recombinants contain a local duplication that is separated by intervening vector sequence, including the selection cassette. In the second step, targeted clones are subjected to negative selection to identify cells that have lost the selection cassette via intrachromosomal recombination between the duplicated sequences. The local recombination event removes the duplication and, depending on the site of recombination, the allele either retains the introduced mutation or reverts to wild type. The end result is the introduction of the desired modification without the retention of any exogenous sequences.

The “double-replacement” or “tag and exchange” strategy—involves a two-step selection procedure similar to the hit and run approach, but requires the use of two different targeting constructs. In the first step, a standard targeting vector with 3′ and 5′ homology arms is used to insert a dual positive/negative selectable cassette near the location where the mutation is to be introduced. After electroporation and positive selection, homologously targeted clones are identified. Next, a second targeting vector that contains a region of homology with the desired mutation is electroporated into targeted clones, and negative selection is applied to remove the selection cassette and introduce the mutation. The final allele contains the desired mutation while eliminating unwanted exogenous sequences.

Site-Specific Recombinases—The Cre recombinase derived from the P1 bacteriophage and Flp recombinase derived from the yeast Saccharomyces cerevisiae are site-specific DNA recombinases each recognizing a unique 34 base pair DNA sequence (termed “Lox” and “FRT”, respectively) and sequences that are flanked with either Lox sites or FRT sites can be readily removed via site-specific recombination upon expression of Cre or Flp recombinase, respectively. For example, the Lox sequence is composed of an asymmetric eight base pair spacer region flanked by 13 base pair inverted repeats. Cre recombines the 34 base pair lox DNA sequence by binding to the 13 base pair inverted repeats and catalyzing strand cleavage and religation within the spacer region. The staggered DNA cuts made by Cre in the spacer region are separated by 6 base pairs to give an overlap region that acts as a homology sensor to ensure that only recombination sites having the same overlap region recombine.

Basically, the site specific recombinase system offers means for the removal of selection cassettes after homologous recombination. This system also allows for the generation of conditional altered alleles that can be inactivated or activated in a temporal or tissue-specific manner. Of note, the Cre and Flp recombinases leave behind a Lox or FRT “scar” of 34 base pairs. The Lox or FRT sites that remain are typically left behind in an intron or 3′ UTR of the modified locus, and current evidence suggests that these sites usually do not interfere significantly with gene function.

Thus, Cre/Lox and Flp/FRT recombination involves introduction of a targeting vector with 3′ and 5′ homology arms containing the mutation of interest, two Lox or FRT sequences and typically a selectable cassette placed between the two Lox or FRT sequences. Positive selection is applied and homologous recombinants that contain targeted mutation are identified. Transient expression of Cre or Flp in conjunction with negative selection results in the excision of the selection cassette and selects for cells where the cassette has been lost. The final targeted allele contains the Lox or FRT scar of exogenous sequences.

Genome editing using recombinant adeno-associated virus (rAAV) platform—this genome-editing platform is based on rAAV vectors which enable insertion, deletion or substitution of DNA sequences in the genomes of live mammalian cells. The rAAV genome is a single-stranded deoxyribonucleic acid (ssDNA) molecule, either positive- or negative-sensed, which is about 4.7 kb long. These single-stranded DNA viral vectors have high transduction rates and have a unique property of stimulating endogenous homologous recombination in the absence of double-strand DNA breaks in the genome. One of skill in the art can design a rAAV vector to target a desired genomic locus and perform both gross and/or subtle endogenous gene alterations in a cell. rAAV genome editing has the advantage in that it targets a single allele and does not result in any off-target genomic alterations. rAAV genome editing technology is commercially available, for example, the rAAV GENESIS™ system from Horizon™ (Cambridge, UK).

Methods for qualifying efficacy and detecting sequence alteration are well known in the art and include, but not limited to, DNA sequencing, electrophoresis, an enzyme-based mismatch detection assay and a hybridization assay such as PCR, RT-PCR, RNase protection, in-situ hybridization, primer extension, Southern blot, Northern Blot and dot blot analysis.

Sequence alterations in a specific gene can also be determined at the protein level using e.g. chromatography, electrophoretic methods, immunodetection assays such as ELISA and western blot analysis and immunohistochemistry.

In addition, one ordinarily skilled in the art can readily design a knock-in/knock-out construct including positive and/or negative selection markers for efficiently g transformed cells that underwent a homologous recombination event with the construct. Positive selection provides a means to enrich the population of clones that have taken up foreign DNA. Non-limiting examples of such positive markers include glutamine synthetase, dihydrofolate reductase (DHFR), markers that confer antibiotic resistance, such as neomycin, hygromycin, puromycin, and blasticidin S resistance cassettes. Negative selection markers are necessary to select against random integrations and/or elimination of a marker sequence (e.g. positive marker). Non-limiting examples of such negative markers include the herpes simplex-thymidine kinase (HSV-TK) which converts ganciclovir (GCV) into a cytotoxic nucleoside analog, hypoxanthine phosphoribosyltransferase (HPRT) and adenine phosphoribosytransferase (ARPT).

According to specific embodiments the agent capable of downregulating MCP1, CCR2 or CCR4 (other targets are described herein in the document, e.g., PD1, P-Selectin and others) is an antibody or antibody fragment capable of specifically binding and inhibiting.

Preferably, the antibody specifically binds at least one epitope of MCP1, CCR2 or CCR4 (other targets are described herein in the document, e.g., PD1, P-Selectin and others).

In another embodiment, the agent is an antibody or antibody fragment capable of specifically binding and inhibiting MCP1, CCR2 or CCR4 (other targets are described herein in the document, e.g., PD1, P-Selectin and others).

As used herein, the term “epitope” refers to any antigenic determinant on an antigen to which the paratope of an antibody binds. Epitopic determinants usually consist of chemically active surface groupings of molecules such as amino acids or carbohydrate side chains and usually have specific three-dimensional structural characteristics, as well as specific charge characteristics.

Methods of producing polyclonal and monoclonal antibodies as well as fragments thereof are well known in the art (See for example, Harlow and Lane, Antibodies: A Laboratory Manual, Cold Spring Harbor Laboratory, New York, 1988, incorporated herein by reference).

The term “antibody” as used in this invention includes intact molecules as well as functional fragments thereof (that are capable of binding to an epitope of an antigen).

As used herein, the term “epitope” refers to any antigenic determinant on an antigen to which the paratope of an antibody binds. Epitopic determinants usually consist of chemically active surface groupings of molecules such as amino acids or carbohydrate side chains and usually have specific three-dimensional structural characteristics, as well as specific charge characteristics.

According to a specific embodiment, the antibody fragments include, but are not limited to, single chain, Fab, Fab′ and F(ab′)₂ fragments, Fd, Fcab, Fv, dsFv, scFvs, diabodies, minibodies, nanobodies, Fab expression library or single domain molecules such as VH and VL that are capable of binding to an epitope of the antigen in an HLA restricted manner.

Suitable antibody fragments for practicing some embodiments of the invention include a complementarity-determining region (CDR) of an immunoglobulin light chain (referred to herein as “light chain”), a complementarity-determining region of an immunoglobulin heavy chain (referred to herein as “heavy chain”), a variable region of a light chain, a variable region of a heavy chain, a light chain, a heavy chain, an Fd fragment, and antibody fragments comprising essentially whole variable regions of both light and heavy chains such as an Fv, a single chain Fv Fv (scFv), a disulfide-stabilized Fv (dsFv), an Fab, an Fab′, and an F(ab′)2, or antibody fragments comprising the Fc region of an antibody.

As used herein, the terms “complementarity-determining region” or “CDR” are used interchangeably to refer to the antigen binding regions found within the variable region of the heavy and light chain polypeptides. Generally, antibodies comprise three CDRs in each of the VH (CDR HI or HI; CDR H2 or H2; and CDR H3 or H3) and three in each of the VL (CDR LI or LI; CDR L2 or L2; and CDR L3 or L3).

The identity of the amino acid residues in a particular antibody that make up a variable region or a CDR can be determined using methods well known in the art and include methods such as sequence variability as defined by Kabat et al. (See, e.g., Kabat et al., 1992, Sequences of Proteins of Immunological Interest, 5th ed., Public Health Service, NIH, Washington D.C.), location of the structural loop regions as defined by Chothia et al. (see, e.g., Chothia et al., Nature 342:877-883, 1989.), a compromise between Kabat and Chothia using Oxford Molecular's AbM antibody modeling software (now Accelrys®, see, Martin et al., 1989, Proc. Natl Acad Sci USA. 86:9268; and world wide web site www(dot)bioinf-org(dot)uk/abs), available complex crystal structures as defined by the contact definition (see MacCallum et al., J. Mol. Biol. 262:732-745, 1996) and the “conformational definition” (see, e.g., Makabe et al., Journal of Biological Chemistry, 283:1156-1166, 2008).

As used herein, the “variable regions” and “CDRs” may refer to variable regions and CDRs defined by any approach known in the art, including combinations of approaches.

Functional antibody fragments comprising whole or essentially whole variable regions of both light and heavy chains are defined as follows:
(i) Fv, defined as a genetically engineered fragment consisting of the variable region of the light chain (VL) and the variable region of the heavy chain (VH) expressed as two chains;
(ii) single chain Fv (“scFv”), a genetically engineered single chain molecule including the variable region of the light chain and the variable region of the heavy chain, linked by a suitable polypeptide linker as a genetically fused single chain molecule.
(iii) disulfide-stabilized Fv (“dsFv”), a genetically engineered antibody including the variable region of the light chain and the variable region of the heavy chain, linked by a genetically engineered disulfide bond.
(iv) Fab, a fragment of an antibody molecule containing a monovalent antigen-binding portion of an antibody molecule which can be obtained by treating whole antibody with the enzyme papain to yield the intact light chain and the Fd fragment of the heavy chain which consists of the variable and CH1 domains thereof;
(v) Fab′, a fragment of an antibody molecule containing a monovalent antigen-binding portion of an antibody molecule which can be obtained by treating whole antibody with the enzyme pepsin, followed by reduction (two Fab′ fragments are obtained per antibody molecule);
(vi) F(ab′)2, a fragment of an antibody molecule containing a monovalent antigen-binding portion of an antibody molecule which can be obtained by treating whole antibody with the enzyme pepsin (i.e., a dimer of Fab′ fragments held together by two disulfide bonds);
(vii) Single domain antibodies or nanobodies are composed of a single VH or VL domains which exhibit sufficient affinity to the antigen; and
(viii) Fcab, a fragment of an antibody molecule containing the Fc portion of an antibody developed as an antigen-binding domain by introducing antigen-binding ability into the Fc region of the antibody.

Methods of producing polyclonal and monoclonal antibodies as well as fragments thereof are well known in the art (See for example, Harlow and Lane, Antibodies: A Laboratory Manual, Cold Spring Harbor Laboratory, New York, 1988, incorporated herein by reference).

Exemplary methods for generating antibodies employ induction of in-vivo production of antibody molecules, screening of immunoglobulin libraries (Orlandi D. R. et al., 1989. Proc. Natl. Acad. Sci. U.S.A. 86:3833-3837; Winter G. et al., 1991. Nature 349:293-299) or generation of monoclonal antibody molecules by continuous cell lines in culture. These include, but are not limited to, the hybridoma technique, the human B-cell hybridoma technique, and the Epstein-Barr virus (EBV)-hybridoma technique (Kohler G. et al., 1975. Nature 256:495-497; Kozbor D. et al., 1985. J. Immunol. Methods 81:31-42; Cote R J. et al., 1983. Proc. Natl. Acad. Sci. U.S.A 80:2026-2030; Cole S P. et al., 1984. Mol. Cell. Biol. 62:109-120).

In cases where target antigens are too small to elicit an adequate immunogenic response when generating antibodies in-vivo, such antigens (haptens) can be coupled to antigenically neutral carriers such as keyhole limpet hemocyanin (KLH) or serum albumin [e.g., bovine serum albumin (BSA)] carriers (see, for example, U.S. Pat. Nos. 5,189,178 and 5,239,078]. Coupling a hapten to a carrier can be effected using methods well known in the art. For example, direct coupling to amino groups can be effected and optionally followed by reduction of the imino linkage formed. Alternatively, the carrier can be coupled using condensing agents such as dicyclohexyl carbodiimide or other carbodiimide dehydrating agents. Linker compounds can also be used to effect the coupling; both homobifunctional and heterobifunctional linkers are available from Pierce Chemical Company, Rockford, Ill. The resulting immunogenic complex can then be injected into suitable mammalian subjects such as mice, rabbits, and the like. Suitable protocols involve repeated injection of the immunogen in the presence of adjuvants according to a schedule which boosts production of antibodies in the serum. The titers of the immune serum can readily be measured using immunoassay procedures which are well known in the art.

The antisera obtained can be used directly or monoclonal antibodies may be obtained as described hereinabove.

Antibody fragments according to some embodiments of the invention can be prepared by proteolytic hydrolysis of the antibody or by expression in E. coli or mammalian cells (e.g. Chinese hamster ovary cell culture or other protein expression systems) of DNA encoding the fragment.

Antibody fragments can be obtained by pepsin or papain digestion of whole antibodies by conventional methods. For example, antibody fragments can be produced by enzymatic cleavage of antibodies with pepsin to provide a 5S fragment denoted F(ab′)2. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly. These methods are described, for example, by Goldenberg, U.S. Pat. Nos. 4,036,945 and 4,331,647, and references contained therein, which patents are hereby incorporated by reference in their entirety. See also Porter, R. R. [Biochem. J. 73: 119-126 (1959)]. Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

As described hereinabove, Fv fragments comprise an association of VH and VL chains. This association may be noncovalent, as described in Inbar et al. [Proc. Nat′l Acad. Sci. USA 69:2659-62 (19720]. Alternatively, the variable chains can be linked by an intermolecular disulfide bond or cross-linked by chemicals such as glutaraldehyde. Preferably, the Fv fragments comprise VH and VL chains connected by a peptide linker. These single-chain antigen binding proteins (sFv) are prepared by constructing a structural gene comprising DNA sequences encoding the VH and VL domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are described, for example, by [Whitlow and Filpula, Methods 2: 97-105 (1991); Bird et al., Science 242:423-426 (1988); Pack et al., Bio/Technology 11:1271-77 (1993); and U.S. Pat. No. 4,946,778, which is hereby incorporated by reference in its entirety.

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells. See, for example, Larrick and Fry [Methods, 2: 106-10 (1991)].

As mentioned, the antibody fragment may comprise a Fc region of an antibody termed “Feab”. Such antibody fragments typically comprise the CH2-CH3 domains of an antibody. Fcabs are engineering to comprise at least one modification in a structural loop region of the antibody, i.e. in a CH3 region of the heavy chain. Such antibody fragments can be generated, for example, as follows: providing a nucleic acid encoding an antibody comprising at least one structural loop region (e.g. Fc region), modifying at least one nucleotide residue of the at least one structural loop regions, transferring the modified nucleic acid in an expression system, expressing the modified antibody, contacting the expressed modified antibody with an epitope, and determining whether the modified antibody binds to the epitope. See, for example, U.S. Pat. Nos. 9,045,528 and 9,133,274 incorporated herein by reference in their entirety.

Humanized forms of non-human (e.g., murine) antibodies are chimeric molecules of immunoglobulins, immunoglobulin chains or fragments thereof (such as Fv, Fab, Fab′, F(ab′).sub.2 or other antigen-binding subsequences of antibodies) which contain minimal sequence derived from non-human immunoglobulin. Humanized antibodies include human immunoglobulins (recipient antibody) in which residues form a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature, 321:522-525 (1986); Riechmann et al., Nature, 332:323-329 (1988); and Presta, Curr. Op. Struct. Biol., 2:593-596 (1992)].

Methods for humanizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers [Jones et al., Nature, 321:522-525 (1986); Riechmann et al., Nature 332:323-327 (1988); Verhoeyen et al., Science, 239:1534-1536 (1988)], by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries [Hoogenboom and Winter, J. Mol. Biol., 227:381 (1991); Marks et al., J. Mol. Biol., 222:581 (1991)]. The techniques of Cole et al. and Boerner et al. are also available for the preparation of human monoclonal antibodies (Cole et al., Monoclonal Antibodies and Cancer Therapy, Alan R. Liss, p. 77 (1985) and Boerner et al., J. Immunol., 147(1):86-95 (1991)]. Similarly, human antibodies can be made by introduction of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10: 779-783 (1992); Lonberg et al., Nature 368: 856-859 (1994); Morrison, Nature 368 812-13 (1994); Fishwild et al., Nature Biotechnology 14, 845-51 (1996); Neuberger, Nature Biotechnology 14: 826 (1996); and Lonberg and Huszar, Intern. Rev. Immunol. 13, 65-93 (1995).

The antibodies described herein may be conjugated to a therapeutic moiety. The therapeutic moiety can be, for example, a cytotoxic moiety, a toxic moiety, a cytokine moiety and a second antibody moiety comprising a different specificity to the antibodies of the invention.

Non-limiting examples of therapeutic moieties which can be conjugated to the antibody of the invention include, but are not limited to, Pseudomonas exotoxin, Diphtheria toxin, interleukin 2, CD3, CD16, interleukin-4, HLA-A2, interleukin-10, amd ricin toxin.

Other therapeutic moieties that may be attached to the antibody of the invention include anticancer agents such as chemotherapeutic agents including but not limited to tubulin inhibitors, such as exatecan, belotecan, Emtansine, etc.

The therapeutic moiety may be attached or conjugated to the antibody of the invention in various ways, depending on the context, application and purpose.

A functional moiety may also be attached to the antibody of the invention using standard chemical synthesis techniques widely practiced in the art [see e.g., hypertexttransferprotocol://worldwideweb (dot) chemistry (dot) org/portal/Chemistry)], such as using any suitable chemical linkage, direct or indirect, as via a peptide bond (when the functional moiety is a polypeptide), or via covalent bonding to an intervening linker element, such as a linker peptide or other chemical moiety, such as an organic polymer. Chimeric peptides may be linked via bonding at the carboxy (C) or amino (N) termini of the peptides, or via bonding to internal chemical groups such as straight, branched or cyclic side chains, internal carbon or nitrogen atoms, and the like. Description of fluorescent labeling of antibodies is provided in details in U.S. Pat. Nos. 3,940,475, 4,289,747, and 4,376,110.

Another agent which can be used along with some embodiments of the invention to downregulate MCP1, CCR2 or CCR4 (other targets are described herein in the document, e.g., PD1, P-Selectin and others) is an aptamer. As used herein, the term “aptamer” refers to double stranded or single stranded RNA molecule that binds to specific molecular target, such as a protein. Various methods are known in the art which can be used to design protein specific aptamers. The skilled artisan can employ SELEX (Systematic Evolution of Ligands by Exponential Enrichment) for efficient selection as described in Stoltenburg R, Reinemann C, and Strehlitz B (Biomolecular engineering (2007) 24(4):381-403).

Another agent capable of downregulating would be any molecule which binds to and/or cleaves MCP1, CCR2 or CCR4 (other targets are described herein in the document, e.g., PD1, P-Selectin and others). Such molecules can be a small molecule, antagonists, or inhibitory peptide.

Another contemplated agent which can be used to downregulate includes a proteolysis-targeting chimaera (PROTAC). Such agents are heterobifunctional, comprising a ligand which binds to a ubiquitin ligase (such as E3 ubiquitin ligase) and a ligand to MCP1, CCR2 or CCR4 (other targets are described herein in the document, e.g., PD1, P-Selectin and others) and optionally a linker connecting the two ligands. Binding of the PROTAC to the target protein leads to the ubiquitination of an exposed lysine on the target protein, followed by ubiquitin proteasome system (UPS)-mediated protein degradation.

The agent (or inhibitor as generally described herein, also referred to herein as “inhibitory agent”) which is used to down-regulate the amount and/or activity of MCP1, CCR2 or CCR4 (other targets are described herein in the document, e.g., PD1, P-Selectin and others) may be formulated for crossing the blood brain barrier.

Exemplary methods for formulating the above described agents to enhance its penetration across the blood brain barrier are described in Yeini et al., Advanced Therapeutics, DOI: 10.1002/adtp.202000124.

Thus, for example, the agents can be formulated in nanoparticles such as liposome-based nanoparticles, amphiphilic micelles, dendrimers, inorganic nanoparticles and polymeric nanoparticles.

Specifically for the delivery of oligonucleotides, the use of cationic nanoemulsions modified biodegradable poly(β-Amino Ester) (PBAE), cell derived extracellular vesicles, spherical nucleic acid nanoparticles, may be considered to improve delivery to the brain.

Since the BBB restricts the passage of most therapeutic agents from the blood to the brain, receptor-mediated transcytosis can offer a non-invasive trafficking system to deliver targeted carriers into the brain parenchyma. In addition, this approach allows selective targeting of tumor cells within the brain tissue, thus reducing toxicity in other tissues and non-tumor cells in the brain. Examples of receptor-mediated approaches include manipulation of the apolipoprotein receptor, targeting of the epidermal growth factor receptor, transferrin receptor targeting, insulin receptor targeting and adhesion molecule targeting are all contemplated.

It will be appreciated that the inhibitors may be directly attached to moieties that target the agent to the blood brain barrier or indirectly (e.g. inhibitory agents may be comprised in a carrier which may be attached to the targeting moieties).

Exemplary inhibitors of MCP-1 or CCR-2 or CCR4 are provided in the Table 1 below.

TABLE 1

| Product name | Product description |
|---|---|
| Carlumab (CNTO888) | Human recombinant monoclonal antibody (type IgG1 kappa) |
| | targeting MCP-1 |
| Bindarit | Oral MCP-1 antagonist 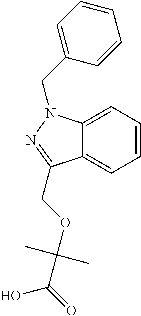 [SMILES: CC(C)(OCc1nn(Cc2ccccc2)c2ccccc12)C(=O)O] |
| mNOX-E36 | MCP-1 antagonist L-enantiomeric RNA oligonucleotide |
| (Spiegelmer) | NOX-E36 (emapticap pegol) binds and neutralizes the human |
| | chemokine MCP-1. |
| S0916 (MLN1202) | CCR2 neutralizing antibody |
| RS504393 | CCR2 antagonist 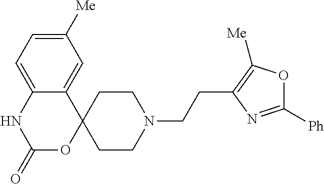 |
| PF-4136309 (INCB8761) | CCR2 antagonist 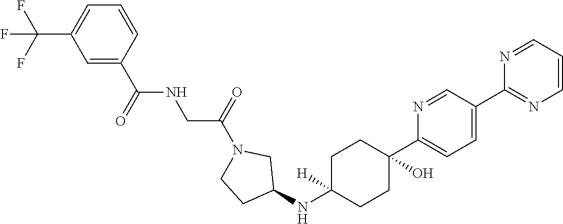 [SMILES: O=C(NCC(=O)N1CC[C@H](N[C@H]2CC[C@@](O)(c3ccc(-c4ncccn4)cn3)CC2)C1)c1cccc(C(F)(F)F)c1] |
| BMS-813160 | CCR2 antagonist 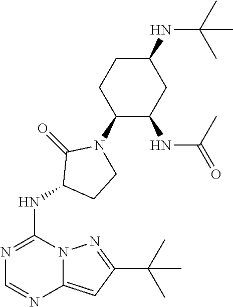 [SMILES: CC(=O)N[C@@H]1C[C@H](NC(C)(C)C)CC[C@@H]1N1CC[C@H](Nc2ncnc3cc(C(C)(C)C)nn23)C1=O] |
| BMS-741672 | CCR2 antagonist 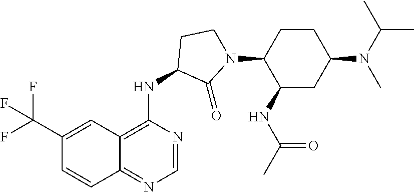 [SMILES: CC(=O)N[C@@H]1C[C@H](N(C)C(C)C)CC[C@@H]1N1CC[C@H](Nc2ncnc3ccc(C(F)(F)F)cc23)C1=O] |
| Cenicriviroc (TAK-652; TBR-652) | CCR2/CCR5 antagonist And HIV-1 antagonist 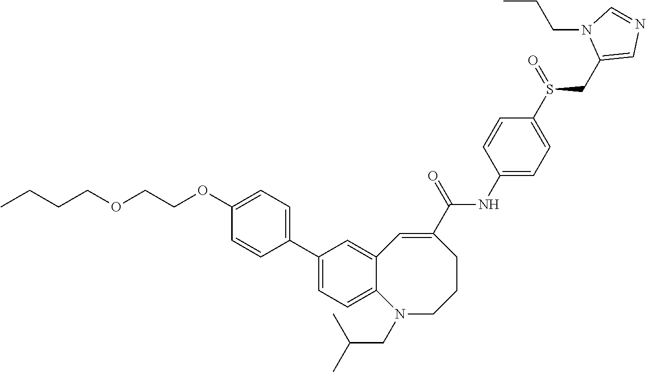 [SMILES: CCCCOCCOc1ccc(-c2ccc3c(c2)/C=C(/C(=O)Nc2ccc([S@@](=O)Cc4cncn4CCC)cc2)CCCN3CC(C)C)cc1] |

Examples of CCR4 inhibitors include but are not limited:
KW-0761 (mogamulizumab)—(Kyowa Hakko Kirin Pharma, Inc.) (ClinicalTrials.gov Identifier: NCT01611142).
RPT193 (RAPT Therapeutics) (ClinicalTrials.gov Identifier: NCT04271514)
AMG 761 (Amgen) (ClinicalTrials.gov Identifier: NCT01514981)
GSK2239633 (GSK) (ClinicalTrials.gov Identifier: NCT01371812) According to a specific embodiment, the method/use comprises a modulator of an immune modulating molecule (e.g., immune inhibitory molecule) or other cancer treatments such as chemotherapy in conjunction with the MCP-1/CCR2/CCR4 inhibitors.

According to a specific embodiment, the advantage of inhibiting MCP-1 is that it will inhibit multiple effectors of the ligand, e.g., CCR2 and CCR4.

According to a specific embodiment, MCP-1, CCR2 or CCR4 and other targets as described herein refer to human forms of the genes or their products.

According to a specific embodiment, the inhibitor is selected from the group consisting of Carlumab, Bindarit and mNOX-E36.

According to a specific embodiment, the small molecule is Binadrit (C₁₉H₂₀N₂O₃).

According to a specific embodiment, the inhibitor is selected from the group consisting of S0916 (MLN1202), RS504393, PF-4136309 (INCB8761), BMS-813160, BMS-741672 and Cenicriviric (TAK-652, TBR-652).

According to a specific embodiment, the inhibitor is an anti MCP-1 antibody, e.g., Carlumab.

The present inventors have found that activating the immune response against brain metastasis by inhibiting the MCP-1/CCR2/CCR4 axis can be improved when administered together (concomitantly, or sequentially e.g., before or after) with a modulator of an immune modulating molecule.

As used herein “an immune inhibitory molecule” also known as “inhibitory checkpoint molecules” refers to molecules which are upregulated by cancer cells or tumor microenvironment that can favour cancer cell escape from immune surveillance. Drugs which are able to block the inhibitory checkpoint molecules have shown efficacy in some types of cancers.

According to an embodiment of the invention, the modulator of the immune inhibitory molecule is an inhibitor.

According to a specific embodiment, the immune inhibitory molecule comprises an immune checkpoint.

According to a specific embodiment, the immune inhibitory molecule is selected from the group consisting of PD-1, PD-L1, P-selectin, P-selectin Ligand-1, CTLA-4, OX-40, IDO, TIGIT, LAG-3 and ICOS.

According to a specific embodiment, the immune inhibitory molecule is a PD-1 (or its ligand) or P-selectin (or its ligand).

P-selectin is a member of the selectin family of adhesion glycoproteins which also includes L- and E-selectins. The selectins mediate the recruitment, initial tethering and rolling, and adherence of leukocytes to sites of inflammation. P-selectin is stored in Weibel-Palade bodies of endothelial cells and alpha-granules of platelets and is rapidly mobilized to the plasma membrane upon stimulation by vasoactive substances such as histamine and thrombin.

P-selectin is a transmembrane glycoprotein (SwissProt sequence P16109) composed of an NH₂-terminal lectin domain, followed by an epidermal growth factor (EGF)-like domain and nine consensus repeat domains. It is anchored in the membrane by a single transmembrane domain and contains a small cytoplasmic tail.

Human P-selectin (also referred to as SELP) has a Uniprot number P16109 and a REFSEQ mRNA NM_003005.4.

P-selectin plays its central role in the recruitment of leukocytes to inflammatory and thrombotic sites by binding to its counter-receptor, P-selectin glycoprotein ligand-1 (PSGL-1) (or a PSGL-1-like receptor on sickled red blood cells), which is a mucin-like glycoprotein constitutively expressed on leukocytes including neutrophils, monocytes, platelets, and on some endothelial cells.

Human PSGL-1 has a Uniprot Number Q14242 and REFSEQ mRNA as set forth in NM_001206609.2 or NM_003006.4.

Thus, the present invention contemplates down-regulating the function of P-selectin by using (1) antibodies to P-selectin, (2) antibodies to PSGL-1, (3) small molecules that mimic the binding domain of PSGL-1, and (4) other molecules that disrupt the binding of P-selectin to PSGL-1. Such agents are further described herein below.

In another embodiment, the agent down-regulates the amount of P-selectin, by reducing expression of P-selectin.

According to a particular embodiment, the P-selectin inhibitor is a monoclonal antibody directed towards PSLGL-1. An example of such an antibody is VTX-0811, which is being developed by Verseau therapeutics (www(dot)verseautx(dot)com/pipeline).

In another embodiment, the P-selectin inhibitor is a small molecule such as rivipansel or tinzaparin, which have been developed to treat sickle cell anemia and as an anticoagulant, respectively. Rivipansel is not specific to P-selectin, but inhibits several members of the selectins family. Tinzaparin is a heparin analogue.

In another embodiment, the P-selectin inhibitor is KF 38789 manufactured by Tocris (3-[7-(2,4-Dimethoxyphenyl)-2,3,6,7-tetrahydro-1,4-thiazepin-5-yl]-4-hydroxy-6-methyl-2H-pyran-2-one).

Additional exemplary P-selectin inhibitors are summarized in Table 2, herein below.

TABLE 2

| Product name | Product description | Phase | References |
|---|---|---|---|
| Crizanlizumab | Humanized monoclonal anti-P-selectin antibody. | II | NCT04611880 (13) |
| (SelG1, | | | |
| Adakveo) | | II | NCT01895361 |
| | | II | NCT04435184 |
| PSI-697 | Oral P-selectin inhibitor. 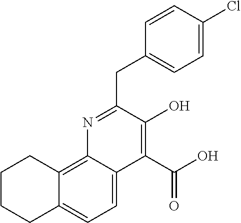 [SMILES: O=C(O)c1c(O)c(Cc2ccc(Cl)cc2)nc2c3c(ccc12)CCCC3] | I | NCT03860506 (14) |
| Cylexin (CY- 1503) | Synthetic analogue of sialyl Lewis X oligosaccharide. 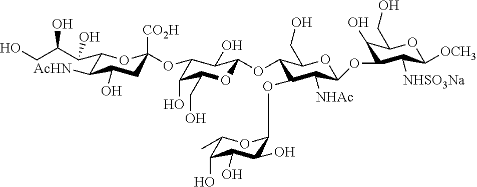 | II, III | NCT00226369 (15) |
| Bimosiamose (TBC1269) | Low-molecular weight sLex-blocking selectin inhibitor that has been shown to inhibit P-selectin-, E-selectin-, and L-selectin- dependent adhesion in humans in vitro and to exert anti- inflammatory effects in different animal models. 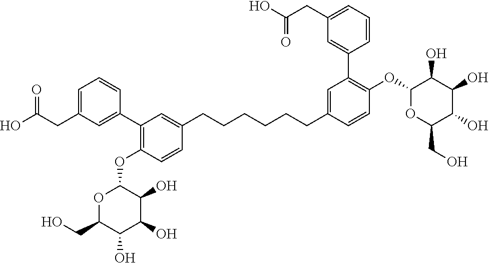 [SMILES: O=C(O)Cc1cccc(-c2cc(CCCCCCc3ccc(O[C@H]4O[C@H](CO)[C@@H](O)[C@H](O)[C@@H]4O)c(-c4cccc(CC(=O)O)c4)c3)ccc2O[C@H]2O[C@H](CO)[C@@H](O)[C@H](O)[C@@H]2O)c1] | II II II | NCT01108913 (16) NCT00962481 NCT00823693 |
| Rivipansel (GMI-1070) | Novel small molecule glycomimetic pan-selectin antagonist. 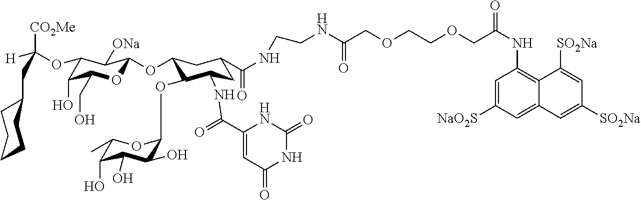 | II, III II | NCT00911495 (17) NCT01119833 |

1. Ataga, K.I., et al., Crizanlizumab for the Prevention of Pain Crises in Sickle Cell Disease. N Engl J Med, 2017. 376(5): p. 429-439.
2. Bedard, P.W., et al., Characterization of the novel P-selectin inhibitor PSI-697 [2-(4-chlorobenzyl)-3-hydroxy-7,8,9,10-tetrahydrobenzo[h] quinoline-4-carboxylic acid] in vitro and in rodent models of vascular inflammation and thrombosis. J Pharmacol Exp Ther, 2008. 324(2): p. 497-506.
3. Park, I.Y., et al., Cylexin: a P-selectin inhibitor prolongs heart allograft survival in hypersensitized rat recipients. Transplant Proc, 1998. 30(7): p. 2927-8.
4. Mayr, F.B., et al., Effects of the pan-selectin antagonist bimosiamose (TBC1269) in experimental human endotoxemia. Shock, 2008. 29(4): p. 475-82.
5. Chang, J., et al., GMI-1070, a novel pan-selectin antagonist, reverses acute vascular occlusions in sickle cell mice. Blood, 2010. 116(10): p. 1779-86.

In one embodiment, the immunomodulatory agent is a checkpoint inhibitor.

By way of example of checkpoint inhibition, detailed here is the PD-1-PD-L1 interaction and its inhibition, but this does not aim to be limiting and is an embodiment of checkpoint inhibition. Thus, a ligand-receptor interaction that has been investigated as a target for cancer treatment is the interaction between the transmembrane programmed cell death 1 protein (PDCD1, PD-1; also known as CD279) and its ligand, PD-1 ligand 1 (PD-L1, CD274). In normal physiology, PD-L1 on the surface of a cell binds to PD1 on the surface of an immune cell, which inhibits the activity of the immune cell. It appears that upregulation of PD-L1 on the cancer cell surface can allow them to evade the host immune system by inhibiting T cells that might otherwise attack the tumor cell. Antibodies that bind to either PD-1 or PD-L1 and therefore block the interaction can allow the T-cells to attack the tumor. In some alternatives, the checkpoint blockade therapeutics comprises anti-PD-1 antibodies or binding fragments thereof (e.g., monoclonal antibodies or humanized versions thereof or binding fragments thereof). In some alternatives, the checkpoint blockade therapeutics comprises PD-L1.

Exemplary anti-PD1 antibodies include Pembrolizumab (Keytruda), Nivolumab (Opdivo), Cemiplimab (Libtayo) and Dostarlimab (Jemperli).

According to a particular embodiment, the anti-PD1 antibody is Nivolumab.

Additional anti-PD1 antibodies include JTX-4014, Spartalizumab (PDR1), Camrelizumab (SHR1210), Sintilimab (I1B1308), Tislelizumab (BGB-A317), Toripalimab (JS 001) INCMGA00012 (MGA012), AMP-224 AMP-514 (MEDI0680).

According to a specific embodiment, the inhibitor of an immune inhibitory molecule is an antibody.

Additional examples of immunomodulatory agents include immunomodulatory cytokines, including but not limited to, IL-2, IL-15, IL-7, IL-21, GM-CSF as well as any other cytokines that are capable of further enhancing immune responses.

Alternatively or additionally, an embodiment of the invention related to administering or using an inhibitor of a cytokine selected from the group consisting of CCL5, GRO-alpha, PAI-1, IL-6 and IL-8/MIP-2.

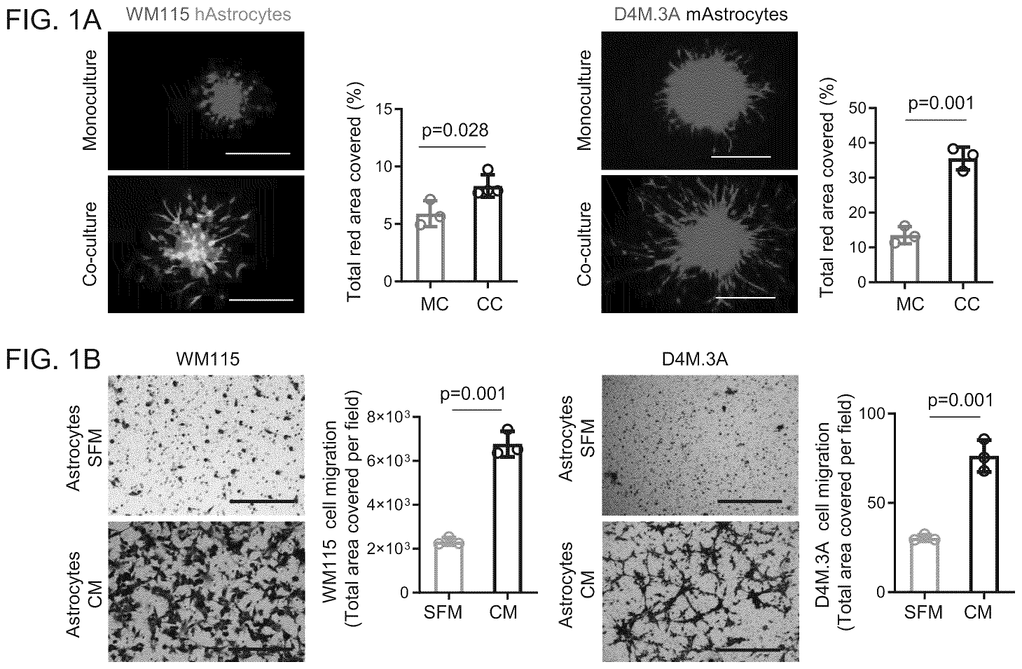

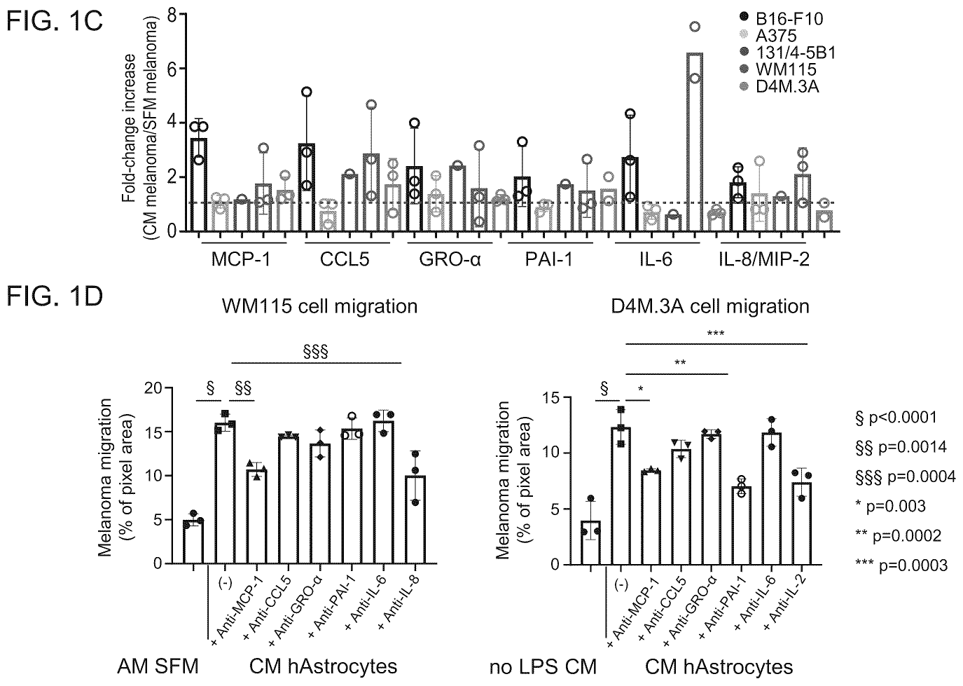

It will be appreciated that an additive effect and even a synergy can be obtained when using in combination of inhibitors of pathways revealed during the screen as evidenced in FIG. 1C. These include, but are not limited to, CCL5, GRO-alpha, PAI-1, IL-6 and IL-8/MIP-2.

Examples of such inhibitors are provided in Table 3 below.

TABLE 3

| Product name | Product description | References |
|---|---|---|
| AZD5069 | Oral CXCR2 antagonist 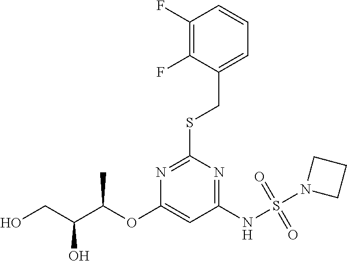 [SMILES: C[C@@H](Oc1cc(NS(=O)(=O)N2CCC2)nc(SCc2cccc(F)c2F)n1)[C@@H](O)CO] | NCT03177187 |
| | AZD-5069 is a potent and selective CXCR2 antagonist with the potential to inhibit neutrophil migration into the airways in patients with chronic obstructive pulmonary disease | NCT01735240 |
| Navarixin (MK-7123; SCH 527123) | Oral CXCR1/2 antagonist Navarixin is a potent, allosteric and orally active antagonist of both CXCR1 and CXCR2 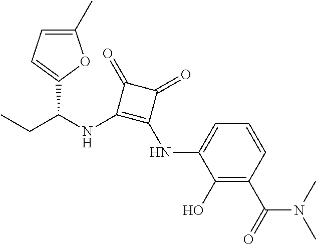 [SMILES: CC[C@@H](Nc1c(Nc2cccc(C(=O)N(C)C)c2O)c(=O)c1=O)c1ccc(C)o1] | NCT03473925 |
| Danirixin | Oral CXCR2 antagonist 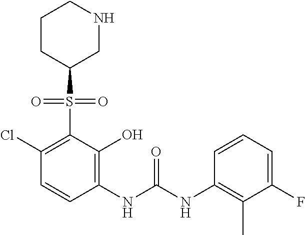 [SMILES: Cc1c(F)cccc1NC(=O)Nc1ccc(Cl)c(S(=O)(=O)[C@H]2CCCNC2)c1O] | NCT03034967 |
| Reparixin | Oral CXCR1/2 antagonist and IL-8 inhibitor 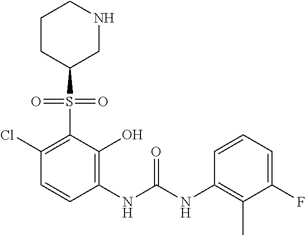 [SMILES: Cc1c(F)cccc1NC(=O)Nc1ccc(Cl)c(S(=O)(=O)[C@H]2CCCNC2)c1O] | NCT04794803 |
| SX-682 | Oral CXCR1/2 antagonist 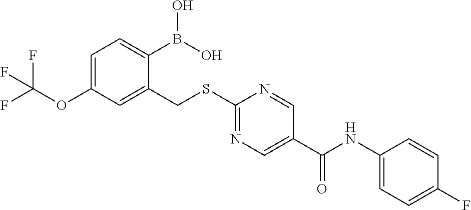 [SMILES: O=C(Nc1ccc(F)cc1)c1cnc(SCc2cc(OC(F)(F)F)ccc2B(O)O)nc1] | NCT03161431 |
| | SX-682 blocks tumor myeloid-derived | NCT04477343 |
| | suppressor cells (MDSCs) | |
| | recruitment and enhance T cell | |
| | activation and antitumor immunity. | |

Target: IL-8

| | | |
|---|---|---|
| BMS-986253 (HuMax- | IL-8 neutralizing antibody | NCT03400332 |
| IL8) | | |
| DF2726A | IL-8 antagonist 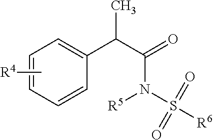 | NCT04123379 (18) |

Target: SEPIN E1

| | | |
|---|---|---|
| Geodon (Ziprasidone) | PAI antagonist 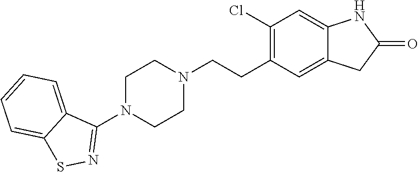 [SMILES: O=C1Cc2cc(CCN3CCN(c4nsc5ccccc45)CC3)c(Cl)cc2N1] | NCT00141271 |

Target: IL-6 receptor (IL-6R)

| | | |
|---|---|---|
| Tocilizumab (Actemra) | IL-6R neutralizing antibody | NCT04320615 |
| | | NCT01194414 |
| | | NCT03863015 |
| Sarilumab (Kevzara) | IL-6R neutralizing antibody | NCT04327388 |
| Satralizumab (SA237) | IL-6R neutralizing antibody | NCT02073279 |

Target: IL-6

| | | |
|---|---|---|
| Siltuximab (SYLVANT) | IL-6 neutralizing antibody | NCT00141271 |
| | | NCT04322188 |

Target: CCR5/CCL5

| | | |
|---|---|---|
| Maraviroc | CCR5 antagonist 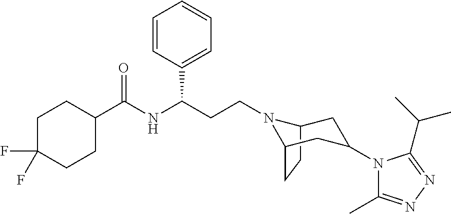 [SMILES: Cc1nnc(C(C)C)n1C1CC2CCC(C1)N2CC[C@H](NC(=O)C1CCC(F)(F)CC1)c1ccccc1] It is a selective, slowly reversible, small molecule antagonist of the interaction between human CCR5 and | NCT04435522 |
| | HIV-1 gp120. It selectively binds to | NCT00098293 |
| | the human chemokine receptor CCR5 | |
| | present on the membrane of CD4 cells | |
| | (T-cells), preventing the interaction | |
| | of HIV-1 gp120 and CCR5 necessary | |
| | for CCR5-tropic HIV-1 to enter cells. | |
| CRISPR/Cas9 CCR5 | CCR5 gene modification | NCT03164135 |
| gene | | NCT03617198 |
| Leronlimab | CCR5 neutralizing antibody | NCT04901689 |

1. Brandolini, L., et al., DF2726A, a new IL-8 signalling inhibitor, is able to counteract chemotherapy-induced neuropathic pain. Scientific Reports, 2019. 9(1): p. 11729.

According to a specific embodiment, the inhibitor is provided with a neoantigen (again, concomitantly or sequentially). Such a cancer neoantigen can be included in a formulation, also referred to as a “nanovaccine”,
WO2020/136657 (incorporated herein by reference) describes such a formulation based on a polymeric nanoparticle comprising:
(i) at least one cancer neoantigen, wherein the cancer neoantigen is encapsulated in the nanoparticle;
(ii) at least one adjuvant;
(iii) a dendritic cell targeting moiety which is attached to the outer surface of the nanoparticle, wherein said dendritic cell targeting moiety is selected from the group consisting of mannose, tri-mannose, PEG-mannose, laminarin and PEG-laminarin; and
(iv) d-α-tocopheryl polyethylene glycol 1000 succinate (TPGS).

As used herein the term “neoantigen” is an epitope that has at least one alteration that makes it distinct from the corresponding wild-type, parental antigen, e.g., via mutation in a tumor cell or post-translational modification specific to a tumor cell. A neoantigen can include a polypeptide sequence or a nucleotide sequence. A mutation can include a frameshift or nonframeshift indel, missense or nonsense substitution, splice site alteration, genomic rearrangement or gene fusion, or any genomic or expression alteration giving rise to a neoORF. A mutation can also include a splice variant. Post-translational modifications specific to a tumor cell can include aberrant phosphorylation. Post-translational modifications specific to a tumor cell can also include a proteasome-generated spliced antigen.

Nanovaccines for the treatment of metastatic brain cancer are well known in the art. For example, a nanovaccine for the treatment of melanoma carried the Melan-A/MART-1(26-35(A27L)) major histocompatibility complex class I (MHCI)-restricted peptide (MHCI-ag) and the Melan-A/MART-1(51-73) MHCII-restricted peptide (MHCII-ag) aimed at the MHC class I and class II antigen presentation pathways, respectively, as previously described (19).

As used herein “the MEK pathway, also known as the MAPK/ERK pathway, is an essential intracellular signaling pathway involved in various cellular processes, including cell growth, proliferation, differentiation, and survival. It stands for Mitogen-Activated Protein Kinase/Extracellular Signal-Regulated Kinase pathway.

The major components of the MEK pathway include: Receptor Tyrosine Kinases (RTKs), growth factors, Ras, Raf, MEK (Mitogen-Activated Protein Kinase Kinase): Also known as MAP2K, ERK (Extracellular Signal-Regulated Kinase): Also known as MAPK, ERK, and transcription factors activated thereby: Once activated, ERK translocates to the nucleus and phosphorylates various transcription factors, leading to changes in gene expression and cellular responses.

As used herein “BRAF” pathway relates to a protein kinase that is a downstream effector of active Ras-GTP. It gets activated when Ras binds to it.

According to a specific embodiment, the inhibitor is provided with an inhibitor of MEK pathway and/or BRAF pathway such as selumetinib (SLM) and dabrafenib (DBF). Trametinib, Binimetinib, Cobimetinib are FDA approved MEK inhibitors.

Other examples of MEK pathway inhibitors include, Trametinib, Cobimetinib, Binimetinib. Other examples of BRAF pathway inhibitors include, Vemurafenib, Encorafenib, LGX818 and PLX8394. Dabrafenib (DBF), Vemurafenib, and Encorafenib are FDA approved BRAF inhibitors.

The MCP1, CCR2 or CCR4 (other targets are described herein in the document, e.g., PD1, P-Selectin and others) inhibitors may be co-formulated with the immunomodulatory agents described herein or any other agent described herein, or may be provided as separate compositions to the subject.

Thus, each agent included in the combination can be formulated separately for use in combination. The drugs are said to be used “in combination” when, in a recipient of both drugs, the effect of one drug enhances or at least influences the effect of the other drug.

The two agents in the combination cooperate to provide an effect on target cells that is greater than the effect of either drug alone. This benefit manifests as a statistically significant improvement in a given parameter of target cell effect. In embodiments, the improvement resulting from treatment with the drug combination can manifest as an effect that is at least additive and desirably synergistic, relative to results obtained when only a single agent is used.

In use, each drug in the combination can be formulated as it would be for monotherapy, in terms of dosage size and form and regimen. In this regard, the synergy resulting from their combined use may permit the use of somewhat reduced dosage sizes or frequencies, as would be revealed in an appropriately controlled clinical trial.

According to one embodiment, the inhibitor and the immunomodulatory agent are administered concomitantly.

According to another embodiment, the inhibitors (e.g., MCP-1/CCR2/CCR4) and the immunomodulatory agent are administered sequentially, wherein the first agent is used, for example, 30 minutes, 1 hour, 6 hours, 12 hours, 24 hours, 48 hours, 72 hours, 96 hours, a week, a month or more after the second agent. Such a determination is well within the capacity of one of skill in the art. In another embodiment, the inhibitor (e.g., MCP-1/CCR2/CCR4) and the immunomodulatory agent are administered sequentially, wherein the second agent is used, for example, 30 minutes, 1 hour, 6 hours, 12 hours, 24 hours, 48 hours, 72 hours, 96 hours, a week, a month or more after the first agent.

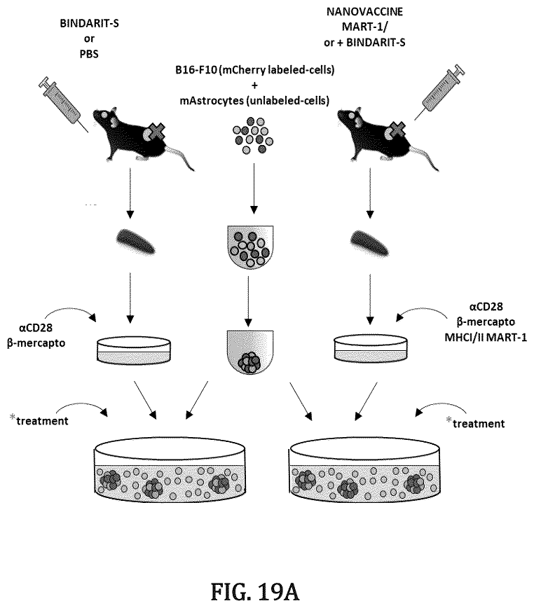

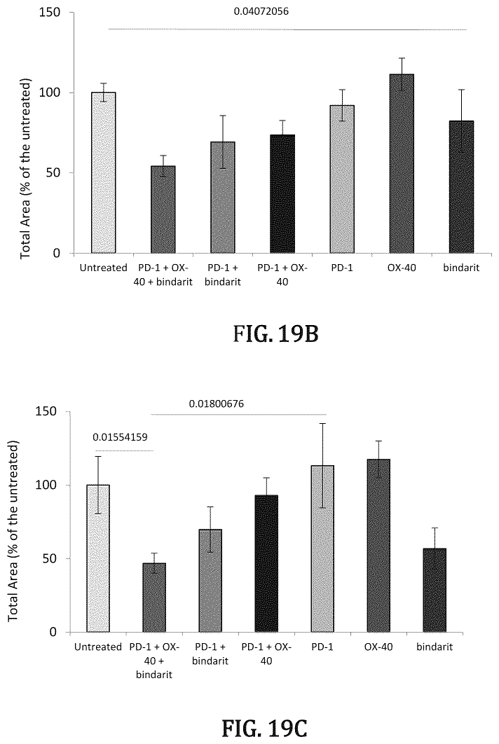

According to a specific embodiment, the nanovaccine is administered prior o administration of inhibitor (e.g., Bindarit and checkpoint inhibitor, e.g., anti-PD1 or anti-OX40 as in FIG. 19A). Regardless the combination of nanovaccine and bindarit, the combined treatments can be administered either simultaneously or the nanovaccine is administered prior bindarit treatment (see Examples section), according to an embodiment of the invention. Regardless the combination of nanovaccine and checkpoint inhibitors, the nanovaccine can be administered prior the treatment with checkpoint inhibitors, according to an embodiment of the invention.

The inhibitors (and immunomodulatory agents) of some embodiments of the invention can be administered to an organism per se, or in a pharmaceutical composition where it is mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the inhibitor accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, PA, latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, intraperitoneal, intranasal, or intraocular injections.

According to a specific embodiment, administering the inhibitor (e.g., to MCP-1/CCR2/CCR4) is peripheral administration.

According to a specific embodiment, administering the inhibitor (e.g., to MCP-1/CCR2/CCR4) is to the central nervous system (CNS).

Conventional approaches for drug delivery to the central nervous system (CNS) include: neurosurgical strategies (e.g., intracerebral injection or intracerebroventricular infusion); molecular manipulation of the agent (e.g., production of a chimeric fusion protein that comprises a transport peptide that has an affinity for an endothelial cell surface molecule in combination with an agent that is itself incapable of crossing the BBB) in an attempt to exploit one of the endogenous transport pathways of the BBB; pharmacological strategies designed to increase the lipid solubility of an agent (e.g., conjugation of water-soluble agents to lipid or cholesterol carriers); and the transitory disruption of the integrity of the BBB by hyperosmotic disruption (resulting from the infusion of a mannitol solution into the carotid artery or the use of a biologically active agent such as an angiotensin peptide). However, each of these strategies has limitations, such as the inherent risks associated with an invasive surgical procedure, a size limitation imposed by a limitation inherent in the endogenous transport systems, potentially undesirable biological side effects associated with the systemic administration of a chimeric molecule comprised of a carrier motif that could be active outside of the CNS, and the possible risk of brain damage within regions of the brain where the BBB is disrupted, which renders it a suboptimal delivery method.

Alternately, one may administer the pharmaceutical composition in a local rather than systemic manner, for example, via injection of the pharmaceutical composition directly into the brain of a patient.

Pharmaceutical compositions of some embodiments of the invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with some embodiments of the invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to some embodiments of the invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multi-dose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water-based solution, before use.

The pharmaceutical composition of some embodiments of the invention may also be formulated in rectal compositions such as suppositories or retention enemas, using, e.g., conventional suppository bases such as cocoa butter or other glycerides.

Pharmaceutical compositions suitable for use in context of some embodiments of the invention include compositions wherein the active ingredients are contained in an amount effective to achieve the intended purpose. More specifically, a therapeutically effective amount means an amount of active ingredients (inhibitor) effective to prevent, alleviate or ameliorate symptoms of a disorder (e.g., inhibitor) or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p.1).

Dosage amount and interval may be adjusted individually to provide levels (e.g. brain levels) of the active ingredient are sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

For any preparation used in the methods of the invention, the dosage or the therapeutically effective amount can be estimated initially from in vitro and cell culture assays. For example, a dose can be formulated in animal models to achieve a desired concentration or titer. Such information can be used to more accurately determine useful doses in humans. Since administration of the disclosed combination is expected to produce improved results over the administration of single agents, the therapeutically effective dose of each of the agents in the combined treatment may be for example less than 50%, 40%, 30%, 20% or even less than 10% the of the FDA approved dose.

For example, therapeutically effective dose of the immunomodulatory agent (e.g. immunomodulatory antibody) in the combined treatment may be for example less than 50%, 40%, 30%, 20% or even less than 10% the of the FDA approved dose. Conversely, the therapeutically effective dose of the inhibitor in the combined treatment may be for example less than 50%, 40%, 30%, 20% or even less than 10% the of the FDA approved dose.

Depending on the severity and responsiveness of the condition to be treated, dosing can be of a single or a plurality of administrations, with course of treatment lasting from several days to several weeks or until cure is effected or diminution of the disease state is achieved.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

### Experimental Procedures

Materials. DMEM, Advanced DMEM, RPMI, MEM media, fetal bovine serum (FBS), and Glutamax were purchased from Gibco (USA). Astrocytes medium (AM) and astrocytes growth supplements were purchased from ScienceCell (USA). EndoGRO Basal Medium and supplements kit (SCME001 kit), Hemacolor® Rapid staining (Cat. No. 1119562500/1119572500) were purchased from Merck (Darmstadt, Germany) Israel. Dulbecco's phosphate buffer saline (PBS), L-glutamine, penicillin, streptomycin, sodium pyruvate, HEPES, MEM non-essential amino acids (NAA) solution, trypsin mycoplasma detection kit, EZ-RNA II total RNA isolation kit and fibronectin (1 mg/ml; Dilution: 1:100) were purchased from Biological Industries Ltd. (Kibbutz Beit HaEmek, Israel). Percoll solution (Cat. No. p4937) lipopolysaccharide (LPS; Cat. No. L8274), 3-(4,5-dimethylthiazol-2-yl)-2,5-diphenyltetrazolinium bromide (MTT; Cat. No. M2128), β-mercaptoethanol (Cat. No. M6250), latex beads for phagocytosis assays (Cat. No. L4655), methyl cellulose (Cat. No. M0512), EDTA (Cat. No. E5134), Triton-100× (Cat. No. 9002931; Lot. No. 033K01501), Dextran, from Leuconostoc spp. (Cat. No. 31390; Lot. 20509147), Tween-20 (Cat. P1379; Lot. No. SLCB6677), Trizma® base (Cat. No. T1503; Lot. No. SLCD9753), acrylamide/bis-acrylamide, 40% solution (Cat. No. A7802; Lot. No. SLBR6765V), formamide (Cat. No. F9037), Evans Blue (Cat. No. 46160; Lot. No. 1185693), and all other chemical reagents, including salts and solvents, were purchased from Sigma-Aldrich (Rehovot, Israel). D-(+)-Sucrose (Cat. No. 001922059100), and Glycine (Cat. No. 000713239100; Lot. No. 1303611), and acetone (Cat. No. 000103020500; Lot. No. 1318371) were purchase from Bio-Labs Itd (Jerusalem, Israel). Milli-Q water was prepared using a Millipore water purification system. Hr-bFGF was purchased from R&D Systems (Minneapolis, Minnesota, USA). Amicon Ultra Centrifugal Filters with 3K molecular weight cut-off (Cat. No. UFC900324), bovine albumin serum (BSA), and sodium dodecyl sulfate (SDS) (Cat. No. 428018; Lot. No. 2956240) were purchased from Merck Millipore (Burlington, Massachusetts, USA). ImageLock tissue culture plate (Cat. No. 4379) were purchased from Sartorious (Gattingen, Germany). Sodium azide (Cat. No. 14314) was purchased from Alfa Aesar (Thermo Fisher Scientific, United Kingdom). SuperSignal™ West Pico Plus chemiluminescent substrate (Cat. No. 34580 Lot. No. UL293523), and Neon™ transfection system kit (Cat. No. MPK1025) were purchased from Thermo Scientific (Rockford, Illinois, USA). Quick DNA miniprep kit (Cat. No. D3024) was purchased from Zymo Research (Irvine, California, USA). PrimeSTAR MAX (Cat. No. R045B) was purchased from Takara Bio (Mountain View, California, USA). T7 Endonuclease 1 (Cat. No. M0302S) was purchased from New England Biolabs (United Kingdom). The qScript™ cDNA Synthesis Kit was purchased from Quantabio (Beverly, Massachusetts, USA). Fast SYBR™ green Master Mix was purchased from Applied Biosystems (California, USA). Collagenase IV, Dispase II (neutral protease) and DNase I, were purchased from Worthington Biochemical Corporation (New Jersey, USA). Rat tail collagen type I, growth factors-reduced (GFR) Matrigel®, 70 μm nylon strainer, and Transwell® permeable support (Cat. No. 3422) were purchased from Corning® (Glendale, Arizona, USA). RBC lysis solution (Cat. No. 420301) anti-mouse/rat/human MCP-1 neutralizing antibody (Cat. No. 505912; Lot. No. B296185; Clone 2H5) were purchased from BioLegend (San Diego, California, USA). MACS MS magnetic columns for cell separation (Cat. No. 130-042-201), CD11b MicroBeads (Cat. No. 130-093-634), and CD31 MicroBeads (Cat. No. 130-097-418) for cell isolation were purchased from Miltenyi Biotec (Bergisch Gladbach, Germany). 2-[(1-Benzyl-1H-indazol-3-yl)methoxy]-2-methylpropanoic (bindarit; Cat No. 130641-38-2) was purchased Angene Chemical (United Kingdom). SELP inhibitor (SELPi) KF38789 (Cat. No. 2748) was purchased from Tocris BioScience (Bristol, United Kingdom). In vivo monoclonal anti-mouse PD-1 antibody (Cat. No. ICH1132, Clone RMP1-14) was purchased from ichorbio (Wantage, United Kingdom). Recombinant human/murine MCP-1 (Cat. No. ADP3; Lot. No. ARL6019071), Human MCP-1 ELISA kit (Cat. No. DCP00), Mouse MCP-1 ELISA kit (Cat. No. MJEOOB) Mouse XL Cytokine Array Kit (Cat. No. ARY028), Human Cytokine Array kit (Cat. No. ARY005B), and Hr-bFGF were purchased from R&D Systems (Minneapolis, Minnesota, USA). Recombinant murine GM-CSF (Cat. No. 315-03; Lot. No. 091855), recombinant human/murine MCP-1 (rh/mMCP-1) (Cat. No. 300-04; Lot. No. 090831), recombinant murine IL-4 (Cat. No. 214-14; Lot. No. 021749), recombinant murine INF-7 (Cat. No. 315-05; lot. No. 061798), rabbit anti-murine JE/MCP-1 (Cat. No. 500-P113; Lot. No. 0608M126RB), rabbit anti-murine KC (CXCL1) (Cat. No. 500-P115; Lot. No. 0909M127RB), rabbit anti-murine MIP-2 (Cat. No. 500-P130; Lot. No. 091CY152RB), rabbit anti-murine RANTES (Cat. No. 500-P118; Lot. No. 0406M124RB), rabbit anti-murine MIP-2 (CXCL2) (Cat. No. 500-P130; Lot. No. 091CY152RB) neutralizing antibodies were purchased from PeproTech (Rehovot, Israel). Anti-mouse SERPIN E1 neutralizing antibody (Cat. No. AM26216PU-N; Lot. No. 27379M0819-A) was purchased from Origene (OriGene Tech. GmbH, Herford, Germany). ProLong® Gold mounting (Cat. No. P36934) and Hoechst 33342 (Cat. No. H3570) were purchased from Invitrogen (Carlsbad, California, USA). Optimal Cutting Temperature (O.C.T.) compound (Cat. No. 4585) was purchased from Scigen Scientific (Gardena, California, USA). Normal goat serum (Cat. No. OORA01661; Lot. No. 28653) was purchased from Aviva Systems Biology Corporation (San Diego, California, USA). Paraformaldehyde (PFA) 16% solution (Cat. No. 15710) was purchased from Electron Microscopy Sciences (Hatfield, Pennsylvania, USA). Mayer's Hematoxylin solution (Cat. No. 05-06002) and Eosin Y solution (Cat. No. 05-10002) were purchased from Bio-Optica (Milano, Italy). Primers and siRNA duplex, Alt-R spCas9 Nuclease V3 Cat. No. 1081059), and Alt-R CRISPR-Cas9 sgRNA were purchased from IDT (Jerusalem, Israel). Polyethyleneimine (PEI) transfecting agent was purchased from Polyplus transfection® (Illkirch, France). Plasmids: mCherry was subcloned by the present inventors into the pQCXIP vector (Clontech, USA) as previously described (20). Protease/phosphatase inhibitor (100×) (Cat. No. 5872S; Lot. No. 19) was purchased from Cell Signaling Technology® (Massachusetts, USA). Magnetol, Gd-DTPA was purchased from Soreq M.R.C. Radiopharmaceuticals (Israel). For animal studies 6-10 week-old male C57BL/6 mice, and 6-8 weeks old male SCID mice were purchased from Envigo CRS (Nes Ziona, Israel).

Antibodies: Primary immunostaining antibodies. Mouse anti-mouse/human MCP-1 (Cat. No. NBP2-22115; Lot. No. B-1; Dilution 1:100), Rabbit anti-mouse/human CCR2 (Cat. No. NBP2-67700; Cat. No. HM0909; Dilution 1:100), Rabbit anti-mouse/human Iba1 (Cat. No. NBP2-19019; Lot. No. 41556; Dilution: 1:200), rabbit anti-mouse/human Ki-67 (Cat. No. NB500-170; Lot. No. G15; Dilution 1:50), Rat anti-mouse F4/80 (BM8, Cat. NBP1-60140; Lot. No. 28094M1219-A; Dilution 1:50 dilution), Rabbit anti-mouse CCR4 (Cat. No. NBP1-86584; Lot. No. B96904), Rat anti-human/mouse PSGL-1 (Cat. No. NB100-78039; Lot. No. C-5; Dilution: 1:50) were purchased from Novus (Colorado, USA). Rat anti-mouse CD274 (B7-H1, PD-L1) (Cat. No. 124318; Lot No. B276997; Clone 10F.9G2; Dilution 1:100), Mouse anti-mouse NK-1.1 (Cat. No. 108701; Lot No. B225451; Clone PK136; Dilution 1:100) was purchased from BioLegend (San Diego, California, USA). Rat anti-mouse CD31 (Cat. No. 550272; Lot. No. 6273859; Dilution 1:25) was purchased from BD Biosciences (Franklin Lakes, NJ, USA). Rat anti-mouse/human CD8 (Cat. No. 14-0808-82; Lot. No. 2003225; Clone 4SMIS; Dilution 1:50) was purchased from eBioscience (San Diego, California, USA). Mouse anti-mouse CD206 (Cat. No. 60143-1-Ig; Lot. No. 10004170; Dilution 1:200), Rabbit anti-mouse/human PD1/CD279 (Cat. No. 60143-1-Ig; Lot. No. 10004170) were purchased from Proteintech (Illinois, USA). Rabbit anti-mouse/human GFAP (Cat. No. Dilution 1:500) was purchased from Dako (Denmark). Rabbit anti-mouse/human CCR4 (Cat. No. PA5-99885; Lot. No. VE2987252; Dilution 1:100) was purchased from Invitrogen (Carlsbad, California, USA). Rat anti-mouse IL-6 (Cat. No. ab191194; Lot. No. GR3186600-3; Clone MP5-20F3; Dilution 1:100) was purchased from Abcam (Cambridge, United Kingdom). Mouse anti-human E/P selectin (Cat. No. BBA1; Clone BBIG-E6 (13D5); Lot. No. ABP0822071; Dilution: 1:30) was purchased from R&D Systems (Minneapolis, Minnesota, USA).

Secondary immunostaining antibodies: Goat anti-mouse Alexa Fluor®647 (Cat. No. ab15115; Lot. No. GR309891-3; Dilution 1:300), goat anti-rabbit Alexa Fluor®488 (Cat. No. ab150077; Lot No. GR315933-2; Dilution 1:300), and goat anti-rabbit Alexa Fluor®647 (Cat. No. Ab150079; Lot. No. Gr3176223-2; Dilution 1:300) were purchased from Abcam (Cambridge, United Kingdom). Goat anti-rat Alexa Fluor®488 (Cat 112-545-068; Lot. No. 143654; Dilution 1:300) and goat anti-rat Alexa Fluor®647 (Cat. No. 112-605-003; Lot No. 137652; Dilution 1:300) were purchased from Jackson ImmunoResearch Laboratories, Inc. (West Grove, Pennsylvania, USA).

Flow cytometry antibodies. Anti-mouse CCR2-APC (Cat. No. 150628; Lot. No. B294911 Clone SA203G11; Dilution 1:100), rat IgG2a, K isotype control-APC (Cat. No. 150628; Lot. No. B294911 Clone SA203G11; Dilution 1:100), anti-mouse FoxP3-Alexa Fluor®647 (Cat. No. 126408; Lot. No. B264076; Clone MF-14; Dilution 1:50), rat IgG2b, K Isotype control-Alexa Fluor® 647 (Cat. No. 400626; Lot. No. B243822; Clone RTK4530; Dilution 1:50), anti-mouse CCR4-PE (Cat. No. 131204; Lot. No. B224241; Clone 2G12; Dilution 1:100), rat IgG2b, K isotype control-PE (Cat. No. 400608; Lot. No. B220932; Clone RTK4530; Dilution 1:100), anti-mouse CD206-PE (Cat. No. 141706; Lot No. B280038; Clone C068C2; Dilution 1:100), mouse IgG1, K isotype control-PE (Cat. No. 400112; Lot. No. B291605; Clone MOPC-21; Dilution 1:100), anti-mouse CD25-FITC (Cat. No. 101907; Lot. No. B276319; Clone 3C7; Dilution 1:50), rat IgG2b, K isotype-control FITC (Cat. No. 400633; Lot. No. B210159; Clone RTK4530, Dilution 1:50), anti-mouse CD11c FITC (Cat. No. 117305; Lot. No. B244373; Clone N418, Dilution 1:100), Armenian hamster IgG isotype control-FITC (Cat. No. 400905; Lot. No. B256163; Clone HTK888, Dilution 1:100), anti-mouse CD107a (LAMP-1)-Alexa Fluor®488 (Cat. No. 121608; Lot. No. B272833; Clone 1D4B; Dilution 1:100), rat IgG2a, K isotype control-Alexa Fluor®488 (Cat. No. 400525; Lot. No. B228070; Clone RTK2758, Dilution 1:100), anti-mouse Ly-6G/Ly-6C (Gr1)-APC (Cat. No. 108411; Lot. No. B262854; Clone RB6-8C5, Dilution 1:100), rat IgG2b, K isotype control-APC (Cat. No. 400611; Lot. No. B261320; Clone RTK4530, Dilution 1:100), anti-mouse/human CD11b-APC/Cyanine7 (Cat. No. 101225; Lot. No. B273856; Clone M1/70, Dilution 1:100), rat IgG2b, K isotype control-APC/Cyanine7 (Cat. No. 400623; Lot. No. B248744; Clone RTK4530, Dilution 1:100), anti-mouse CD3F PerCP/Cyanine5.5 (Cat. No. 100328; Lot. No. B281044; Clone 145-2C11; Dilution 1:50), and Armenian hamster IgG isotype control (Cat. No. 400931; Lot. No. B247913; Clone HTK888; Dilution 1:50) were purchased from BioLegend (San Diego, California, USA). Anti-mouse CD3-FITC (Cat. No. 130-119-798; Lot. No. 5190919162; Clone REA641; Dilution 1:50), anti-mouse F4/80-FITC (Cat. No. 130-117-509; Lot. No. 52003066886; Clone REA126; Dilution 1:50), REA Control-FITC (Cat. No. 130-113-449; Lot No. 5190711318; Clone REA293; Dilution 1:50), anti-mouse CD8b-APC (Cat. No. 130-111-712; Lot No. 5190919051; Clone: REA793; Dilution 1:50), CD68-APC REAfinity™ (Cat. No. 130-112-857; Lot No. 5200805639; Clone: REA835; Dilution 1:50) REA Control-APC (Cat. No. 130-113-446; Lot. No. 5190711317; Clone REA293; Dilution 1:50), Rat IgG2a isotype control-APC (Cat. No. 130102655; Lot. No. 5191030093; Dilution 1:10), anti-mouse CD38-APC-Vio®770 (Cat. No. 130-125-227; Lot No. 5200405654; Clone REA616; Dilution 1:50), REA Control-APC-Vio®770 (Cat. No. 130-113-447; Lot No. 51909191419; Clone REA293; Dilution 1:50) anti-mouse CD4 VioBlue® (Cat. No. 130-118-696; Lot No. 5190919087; Clone REA605; Dilution 1:50), anti-mouse MHC Class II-VioBlue® (Cat. No. 130-123-278; Lot. No. 5200805636; Clone M5/114.15.2; Dilution 1:50), anti-mouse CD3c VioBlue® (Cat. No. 130118849; Lot. No. 5200805619; Clone 17A2; Dilution 1:50), anti-mouse VioBlue® CD11b REAfinity™ (Cat. No. 130113810, Lot. No. 5200805605; Clone REA592; Dilution 1:50), REA Control-VioBlue® (Cat. No. 130-113-545; Lot. No. 5190711335; Clone REA293; Dilution 1:50), anti-mouse CD11b-PE-Vio®770 (Cat No. 130-113-808; Lot No. 5190919070; Clone REA592; Dilution 1:50), and REA Control-PE-Vio®770 (Cat. No. 130-113-452; Lot No. 5191025178; Clone REA293; Dilution 1:50) were purchased from Miltenyi Biotec (Bergisch Gladbach, Germany). Western Blot antibodies: Anti-human total p65 (Cat. No. D14E12; Lot. No. 13; Dilution 1:1000), anti-human phosphor-p65 (Cat. No. S536; Lot. No. 16; Dilution 1:1000), anti-human vinculine (Cat. No. E1E9V; Lot. No. 6; Dilution 1:1000) and anti-rabbit HRP (Cat. No. 7074P2; Lot. No. 28; Dilution 1:2000) were purchased from Cells Signaling Technology® (Massachusetts, USA).

Cell cultures: Human melanoma cell lines. Metastatic melanoma 131/4-5B1 cells (kindly provided by Robert Kerbel) (21) were cultured in RPMI medium supplemented with 10% FBS, 100 IU/mL Penicillin, 100 μg/mL Streptomycin, 12.5 IU/mL Nystatin, 2 mM L-glutamine.

Primary melanoma A375 cells (ATCC, USA) were cultured in RPMI medium supplemented with 10% FBS, 100 IU/mL Penicillin, 100 μg/mL Streptomycin, 12.5 IU/mL Nystatin, 2 mM L-glutamine, 1 mM sodium pyruvate, and 25 mM HEPES. Primary melanoma WM115 cells (ECACC, Porton Down, Salisbury, UK) were cultured in MEM medium supplemented with 10% FBS, 100 IU/mL Penicillin, 100 g/mL Streptomycin, 12.5 IU/mL Nystatin, and 2 mM L-glutamine, 1 mM sodium pyruvate and 1× of MEM NAA.

Murine melanoma cell lines. Primary melanoma B16-F10 cells (ATCC, USA) were cultured in DMEM supplemented with 10% FBS, 100 U/mL Penicillin, 100 g/mL Streptomycin, 12.5 IU/mL Nystatin, and 2 mM L-glutamine. Primary melanoma B2905 (kindly provided by Glenn Merlino) were grown in RPMI supplemented with 10% FBS, 100 U/mL Penicillin, 100 g/mL Streptomycin, 12.5 IU/mL Nystatin, 25 mM HEPES, and 2 mM L-glutamine. Primary melanoma Mel-ret cells (kindly provided by Neta Erez) were grown in RPMI supplemented with 10% FBS, 100 U/mL Penicillin, 100 g/mL Streptomycin, 12.5 IU/mL Nystatin, 1 mM sodium pyruvate, and 2 mM L-glutamine. Primary melanoma D4M.3A cells (kindly provided by David W. Mullis) (22) were grown in Advanced DMEM supplemented with 5% FBS, 100 IU/mL Penicillin, 100 g/mL Streptomycin, 12.5 IU/mL Nystatin, and 2 mM Glutamax. All melanoma cell lines were labeled with pQC-mCherry retroviral particles, as previously described (20).

Human breast cancer cell lines. Human breast adenocarcinoma MDA-MB-231 (ATCC, USA) were cultured in DMEM medium supplemented with 10% FBS, 100 IU/mL Penicillin, 100 μg/mL Streptomycin, 12.5 IU/mL Nystatin, 2 mM L-glutamine.

Murine breast cancer cell lines. Murine mammary adenocarcinoma 4T1 cells (ATCC, USA) were cultured in RPMI 1640 medium supplemented with 10% FBS, 100 U/mL Penicillin, 100 μg/mL Streptomycin, 12.5 U/mL Nystatin, 2 mM L-glutamine, 42 mM Glucose 45%, 1 mM Sodium Pyruvate and 10 mM HEPES buffer. MDA-MB-231 and 4T1 cell lines were labeled with pQC-mCherry retroviral particles, as previously described (20).

Human brain stromal cell. Human astrocytes (ScienceCell, USA) were cultured in AM supplemented with 2% FBS, 100 IU/mL Penicillin, 100 μg/mL Streptomycin, and 1% astrocytes growth supplements.

Murine brain stromal cell. Freshly isolated murine astrocytes were cultured in AM supplemented with 2% FBS, 100 IU/mL Penicillin, 100 μg/mL Streptomycin, and 1% astrocytes growth supplements. Freshly isolated mBEC cells were grown in EndoGRO-LS supplemented with t 0.2%, rh EGF 5 ng/mL, Ascorbic Acid 50 μg/mL, L-Glutamine 10 mM, Hydrocortisone Hemisuccinate 1 μg/mL, Heparin Sulfate 0.75 U/mL, FBS 2%, 100 U/mL Penicillin, 100 g/mL Streptomycin, 12.5 U/mL Nystatin and 1 ng/ml Hr-bFGF. Bone marrow freshly isolated cells were cultured in RPMI medium supplemented with 10% FBS, 100 IU/mL Penicillin, 100 g/mL Streptomycin, 12.5 IU/mL Nystatin, and 2 mM L-glutamine, 1 mM sodium pyruvate, 1× of MEM NAA solution, and 50 μM β-mercaptoethanol.

Cells were routinely tested for mycoplasma contamination with a mycoplasma detection kit (Biological Industries, Israel). All the cell cultures were grown at 37° C. in 5% CO₂.

Brain stromal cell isolation. Brains from 6-10 week-old male C57BL/6 or female BALB/c mice were harvested, chopped and incubated in rotation with Collagenase III\Dispase solution for 50 min at 37° C. Red blood-cells lysis was carried out followed by Percoll gradient for myelin separation. Cell suspension was then incubated with CD11b and CD31 microbeads for microglia and endothelial cells isolation, respectively, then placed with the microbeads in MACS MS magnetic columns. Isolated microglia were discarded while endothelial cells were plated in 96 well plates or Transwell® chambers following well-surface coating with 0.2 mg/ml rat tail type I collagen. The negative population, enriched in astrocytes, was collected following beads separation, plated in a 10 cm² dish and grown in AM. After cells separation, each of the populations was evaluated for cell type and purity by FACS (Attune NxF, Life Technologies, USA).

Splenocytes isolation. Splenocytes were freshly isolated from the spleens of healthy 7-11 weeks old C57BL/6 mice, or from mice bearing PM. Spleens were mashed and passed through a 70 μm nylon strainer, followed by RCB lysis. Flasks were coated with anti-CD3F (Cat. 100340; Lot. B302116; Clone 145-2C11) antibody, and splenocytes (30×10⁶ cells/75 cm² flask) were incubated with 2 μg/ml anti-CD28 (Cat. 102116; Lot. B331922; Clone 3751), and 10 U/ml rhIL-2 for 6 days. Medium was replaced 3 days after cells isolation and 3 days later, activated splenocytes (from healthy or MBM bearing mice) were co-cultured for 48 h with MBM 3D spheroids in Matrigel®.

Astrocytes Conditioned-Medium (CM). To generate astrocytes medium enriched with secreted cytokines, half a million of human astrocytes were grown in serum-free astrocyte medium (AM SFM) for 24 h. One million of freshly isolated murine astrocytes were incubated in AM SFM for 24 h supplemented with 100 ng/mL LPS to stimulate MCP-1 expression. As previously shown by van Neerven et al., (23) it is necessary to pre-stimulate murine astrocytes culture for 24 h to ensure activation and secretion of cytokines secretion to the media. CM was then collected and filtered through a 0.2 μm pores-sized filter to remove cell debris.

Cytokine array. Human or murine astrocytes and melanoma cells (131-4/5B1, A375, WM115, B16-F10 and D4M.3A) were grown for 24 h in their own SFM. Astrocytes and melanoma cells were then incubated for 24 h in melanoma and astrocytes CM, respectively. Twenty-four hours later, medium was replaced by SFM in both mono-cultures for additional 24 h and then collected. As negative control the present inventors included melanoma cells and astrocytes grown in their own CM. Human or murine astrocytes (2×10⁵) and breast cancer cells (1×10⁵) (MDA-MB-231; 4T1) were grown in AM, either separately or in co-culture for Seventy-two hours. Media were collected, filtered with 0.2 μm pores-sized filter, concentrated (Amicon® Ultra 15 centrifugal filter) and analyzed for cytokines secretion using Cytokine Array kit according to the protocol provided by the manufacturer.

mCherry FACS sorting. To assess the changes in astrocytes gene signature following interaction with melanoma cells, human or murine astrocytes were seeded (1×10⁶ cells/10 cm² plate) and 24 h after mCherry-labeled 131/4-5B1, WM115, B16-F10, D4M.3A, B2905 cells, respectively, were added to the astrocytes culture (0.5×10⁶ cells/10 cm² plate). Alternatively, astrocytes were grown alone in AM SFM for 24 h. Cells were detached and washed using PBS buffer supplemented with 2 mM EDTA and 0.5% BSA, and 0.1% sodium azide (FACS buffer) and sorted by discarding mCherry-labeled melanoma cells by FACS (AriaIII, BD Bioscience, USA) using mCherry channel (635 nm). RNA was extracted from astrocytes for gene expression analysis.

Drugs preparation. For in vitro experiments, a stock solution of 100 mM bindarit was prepared in DMSO, and serial dilutions (0.001, 0.01, 0.1, 0.3, 0.5, 1 mM) were made using the culture medium. For in vivo experiment, 150 mg bindarit was dissolved in 0.5 M NaHCO₃ and water, and then lyophilized overnight. A stock of 100 mg/kg bindarit was prepared in PBS.

For in vitro experiments, a stock solution of 1 mM SELPi was prepared in DMSO, and was further diluted to 0.5 μM in the culture medium. For in vivo experiments, SELPi (0.8 mg/ml) was dissolved in DMSO (0.01%), polyethyleneglycol (93.2 mg/ml), Tween-80 (14.8 mg/ml), and DDW.

MTT assay. D4M.3A cells (750 cells/well), B16-F10 cells (750 cells/well), WM115 cells (2×10³ cells/well), or A375 cells (1×10³ cells/well) were plated onto 96-well plates and incubated for 24 h. Cells were then treated with bindarit in serial dilutions. Cells metabolic activity MTT assay was used to determine the IC₅₀. Seventy-two hours following treatment, 30 μl of 3 mg/ml MTT solution in PBS was added to the medium for 5 h. The medium was then discarded and 100 μl DMSO was added to dissolve the formazan crystals formed. Absorbance was measured at 560 nm using SpectraMax M5 plate reader (Molecular Devices, San Jose, California, USA).

Hemolysis Assay. Fresh blood was obtained from a male Wistar rat (˜250 g) by cardiac puncture and collected in a heparinized tube. Blood was then centrifuged at 1000×g for 10 min at 4° C. Supernatant was discarded and erythrocytes were washed 3 times with pre-chilled PBS. Then a 2% w/w RBC stock solution (2% RBC in cold PBS) was loaded into a 96-well plate and incubated with serial dilutions (0.31-2.5 mg/ml) of bindarit for 1 h at 37° C. Following plate centrifugation, the supernatants were transferred to a new plate and absorbance was measured at 550 nm using a SpectraMax M5 plate reader (Molecular Devices, San Jose, California, USA). SDS and Triton-X100 were used as positive controls, whereas 70 kDa Dextran was used as a negative control.

Transwell® Migration Assay. For testing melanoma migration towards astrocytes-secreted cytokines enriched medium, melanoma 131/4-5B1 or D4M.3A (1×10⁵ cells/insert), WM115 or B16-F10 cells (5×10⁴ cells/insert) were seeded in 8 μm inserts of a Transwell® coated with 10 μg/mL bovine fibronectin. Astrocytes SFM or CM was loaded in the lower chamber and migration of the melanoma cells towards SFM or CM was monitored for 12 to 48 h. In order to assess melanoma migration in co-culture with astrocytes, human astrocytes (1×10⁵ cells/well) or murine astrocytes (2×10⁵ cells/well) were seeded in the lower chambers of the Transwell® inserts. Astrocytes SFM alone or supplemented with 0.35 mM bindarit was added to growing astrocytes in the lower chamber. Melanoma cells (131/4-5B1, A375, WM115, D4M.3A, or B16-F10) were then allowed to migrate towards untreated or treated astrocytes for 12 to 48 h.

D4M.3A cells were allowed to migrate towards astrocytes' CM collected from treated astrocytes (0.35 mM bindarit) or towards astrocytes CM supplemented with freshly added 0.35 mM bindarit, as additional control to exclude cytotoxic effects of the bindarit on the migrating cells.

In order to evaluate melanoma migration towards astrocytes CM depleted of specific cytokines, murine (B16-F10 5×10⁴ cells/well or D4M.3A—1×10⁵ cells/well) and human (WM115 or 131/4-5B1—5×10⁴ cells/well) melanoma cells were seeded in the upper chambers of the Transwell®. Astrocytes SFM, astrocytes CM alone or supplemented with neutralizing antibodies (RANTES, MCP-1, GRO-α, or SERPIN-E1), were loaded in the lower chamber. Melanoma cells were then allowed to migrate towards the astrocytes CM supplemented with neutralizing antibodies for the selected cytokines for 12 to 24 h. For the D4M.3A and B16-F10 migration assays, astrocytes were previously activated with LPS for 24 h prior CM collection, as described before. Anti-human neutralizing antibodies for RANTES (0.1 μg/ml), MCP-1 (0.2 μg/ml), GRO-α (0.1 μg/ml), SERPIN E1 (0.1 μg/ml), IL-6 (0.4 μg/ml), IL-8 (0.2 μg/ml) were used according to the manufacturer's protocol. Anti-mouse neutralizing antibodies for RANTES (0.6 μg/ml), MCP-1 (12 μg/ml), GRO-α (0.5 μg/ml), SERPIN E1 (4 μg/ml), IL-6 (0.016 μg/ml), MIP-2 (1.8 μg/ml), were used according to the manufacturer's protocol.

For testing breast cancer migration towards astrocytes-secreted cytokines enriched medium, MDA-MB-231 cells (1×10⁵ cells/insert) were seeded in 8 μm inserts of a Transwell® coated with 10 μg/mL bovine fibronectin. Astrocytes SFM, CM or CM supplemented with 0.3 mM bindarit, were loaded in the lower chamber and migration of the breast cancer cells towards SFM or CM was monitored for 24 h.

In order to evaluate breast cancer migration towards MCP-1 protein, MDA-MB-231 cells were seeded in the upper chambers of the Transwell®. Astrocytes SFM alone or supplemented with Recombinant human MCP-1, were loaded in the lower chamber. Breast cancer cells were then allowed to migrate for 24 h.

All migrated cells were then fixed and stained (Hema 3 Stain System; Fisher Diagnostics, fixed and stained (Hema 3 Stain System; Fisher Diagnostics, USA). The stained migrated cells were imaged using EVOS FL Auto microscope (Life Technologies, USA) using 10× objective, brightfield illumination.

For transendothelial migration, freshly isolated mBEC cells (4×10⁵ cells/insert) were seeded and grown for 96 h in 8 μm Transwell® inserts coated with 0.2 mg/ml collagen type I. mCherry-labeled D4M.3A or B16-F10 cells were seeded on 100 ng/ml collagen type I added on top of mBEC. Melanoma cells were allowed to migrate for 48 h towards treated astrocytes (0.35 mM bindarit) or 0.35 mM bindarit pre-treated astrocytes CM. Migrated cells were then fixed, and the nuclei were stained with Hoechst solution in PBS (1:5000). Migrated mCherry-labeled cells were imaged using EVOS FL Auto microscope (Life Technologies, USA) using 10× objective, Tex-Red LED Cube illumination. Migrated cells from the captured images per membrane were analyzed using ImageJ 1.52v software.

siRNA MCP-1 silencing. Human astrocytes (4×10⁴ cells/ml/well) were seeded onto 6-well plates and 24 h later transfected with polyplexes containing PEI and MCP-1 siRNA (NM_002982). siRNA-targeting MCP-1 (200 or 500 nM), or negative control siRNA (NC 500 nM) were incubated with PEI for 20 min prior cells treatment, according to manufacturer's protocol. Twenty-four hours later cells were collected, and total RNA was extracted.

siRNA treatment and astrocytes viability. Human astrocytes (4×10⁴ cells/ml/well) were seeded in 24-well plates and 24 h later transfected with polyplexes containing PEI:siRNA at 200 nM or 500 nM siRNA targeting MCP-1 or NC. Twenty-four to seventy-two hours later cells were detached using trypsin and counted using Coulter cells counter (Beckman, USA).

mRNA isolation and qPCR. Total RNA was isolated using EZ-RNA II total RNA isolation kit according to the manufacturer's protocol. Briefly, samples were lysed with 0.5 mL denaturing solution/6-well culture plate. Water saturated phenol was added, and samples were centrifuged. Isopropanol was then added to the aqueous colorless (upper) phase to precipitate RNA, and samples were centrifuged once more. RNA pellet was washed with 75% ethanol, centrifuged, and resuspended with ultra-pure double distilled water. RNA concentration was evaluated by measuring absorption (A260/A280) using NanoDrop® ND-1000 Spectrophotometer according to the manufacturer's V3.5 user's manual (Nano-Drop Technologies, Wilmington, DE). Reverse transcription reaction for mRNA was performed using qScript™ cDNA Synthesis Kit for RT-PCR, following the manufacturer's protocol. Briefly, 1 μg of total RNA sample was mixed with qScript Reverse Transcriptase, dNTPs and nuclease free water. The reaction tube was then incubated at 42° C. for 30 min and heated at 85° C. for 5 min to stop cDNA synthesis reaction. cDNA levels were quantified using custom qPCR primers and normalized to GAPDH or HPRT housekeeping genes. qPCR for cDNAs was performed using Fast SYBR™ green Master Mix, according to manufacturer's instructions using StepOnePlus real-time PCR system (Applied Biosystems, Thermo Fisher, USA). Human primers: Human MCP-1 Forward: 5′-GGCTGAGACTAACCCAGAAAC-3′ (SEQ ID NO: 1); reverse: 5′-GAATGAAGGTGGCTGCTATGA-3′ (SEQ ID NO: 2). Murine primers: Mouse MCP-1 Forward: 5′-TGTGTTCATCCCCAGAACCG-3′(SEQ ID NO: 3); reverse: 5′GGGTACAGTTCCTTGGAGCC-3′(SEQ ID NO: 4). Mouse HPRT Forward: 5′-TGATTATGGACAGGACTGAAAGA-3′ (SEQ ID NO: 5); reverse: 5′-GCAGGTCAGCAAAGAACTTATAG-3′ (SEQ ID NO: 6).

Human/murine primers: human/mouse GAPDH Forward: 5′-ATTCCACCCATGGCAAATTC-3′(SEQ ID NO: 7); reverse: 5′-GGATCTCGCTCCTGGAAGATG-3′(SEQ ID NO: 8).

ELISA. MCP-1 levels secreted by astrocytes were assessed by ELISA using the Human CCL2/MCP-1 Quantikine® or the Murine CCL2/MCP-1 Quantikine®. Activation of human or murine astrocytes was achieved by growing 8×10⁴ cells/6 wells-plate in astrocytes SFM (with LPS in case of murine cells) for 24 h. Inhibition of human or murine astrocytes-secreted MCP-1 was achieved by growing 8×10⁴ cells/6 wells-plate in astrocytes SFM supplemented with 0.35 mM bindarit (with LPS in case of murine cells). Twenty-four hours later the medium was collected, filtered with 0.2 μm filter, and analyzed for MCP-1 secretion. Alternatively, human astrocytes (4×10⁴ cells/6 wells-plate) were grown in astrocytes medium supplemented with 2% serum for 24 h. PEI complexed with MCP-1 siRNA and NC siRNA were added to the medium. Twenty-four hours following treatment, the cells were incubated in AM SFM for additional 24 h. Medium was then collected, filtered through 0.2 m-pore membrane and analyzed for MCP-1 secretion levels. To assess MCP-1 secretion in melanoma-activate astrocytes, human melanoma WM115 cells or murine melanoma D4M.3A (5×10⁵ cells/10 cm² dish) were grown in melanoma SFM. Twenty-four hours later, melanoma CM was collected, filtered with 0.2 μm filter and added to growing human and murine astrocytes (2.5×10⁵ or 5×10⁵ cells/10 cm² dish), respectively. Twenty-four hours later astrocytes CM was collected, filtered with 0.2 μm filter and analyzed for MCP-1 secretion. CM of melanoma cells was used to determine the basal MCP-1 melanoma containing-medium prior to astrocytes enrichment secretion.

MCP-1 levels secreted by breast cancer cells and astrocytes were assessed by ELISA using the Human CCL2/MCP-1 Quantikine® or the Murine CCL2/MCP-1 Quantikine®. Human or murine astrocytes (2×10⁵) and breast cancer cells (1×10⁵) (MDA-MB-231; 4T1) were grown in AM, either separately or in co-culture. Seventy-two hours later the medium was collected, filtered with 0.2 μm filter, and analyzed for MCP-1 secretion.

Wound Healing Assay. To study the role of MCP-1 in mediating melanoma cells migration, (WM115 and 131/4-5B1 cells) (5×10⁴ cells/well) were plated onto 96-well ImageLock tissue culture plate and allowed to grow until confluence. Next, a wound was created in each well using a 96-pin wound-making tool (WoundMaker, Essen BioScience, Sartorius, USA), and dislodged cells were washed with PBS. Melanoma cells were treated for 48 h with astrocytes CM, human astrocytes CM treated with siRNA:PEI polyplexes (500 nM siRNA targeting MCP-1 or scramble siRNA), astrocytes CM supplemented with 10 g/mL anti-MCP-1 neutralizing antibody, astrocytes SFM supplemented with 1 μg/mL rh/mMCP-1. To monitor wound closure, the plate was placed in the IncuCyte™ ZOOM Live Cell Imaging system (Essen Bioscience, Sartorius, USA) and phase contrast images were taken every 2 h intervals over a course of 48 h using 10× objective. Results were calculated by the IncuCyte™ Software and presented as relative wound density (relative to the background density of the wound at time 0).

Melanoma proliferation in co-culture. Human and murine melanoma mCherry-labeled cells (WM115—2×10³ cells/well, A375—1×10³ cells/well, D4M.3A—750 cells/well, B16-F10—1×10³ cells/well) were plated alone onto 96-well culture plates in their growing medium for 24 h. In addition, melanoma cells were co-cultured with human or murine astrocytes at a ratio of 1:1 in AM. Twenty-four hours later, astrocytes SFM was added to the cell mono-culture or the co-culture. Cells' growth was monitored for 48 h by IncuCyte™ ZOOM Live Cell Imaging system (Essen BioScience, Sartorius, USA) and red phase images (representing mCherry-labeled melanoma cells) were taken every two hours using 10× objective. Results were calculated by the IncuCyte™ Software and presented as total red area covered (image/well) normalized to time zero for each cell cultures.

Cells' growth was monitored for 96 h by IncuCyte™ ZOOM Live Cell Imaging system (Essen BioScience, Sartorius, USA) and red phase images (representing mCherry-labeled breast cancer cells) were taken every two hours using 10× objective. Results were calculated by the IncuCyte™ Software and presented as total red area covered (image/well) normalized to time zero for each cell cultures.

Multicellular tumor spheroids (MCTS). MCTS were prepared using the hanging-drop method as previously shown (24). Briefly, 3D tumor spheroids were formed from a mixture of melanoma with brain stromal microenvironment cells. Human or murine mCherry-labeled melanoma cells (WM115, A375, D4M.3A, B16-F10, B2905—4×10⁵ cells/ml) alone or in combination with human GFP-labeled or unlabeled murine astrocytes (4×10⁵ cells/ml—in ratio 1:1) were grown in AM supplemented with 0.24 w/v % methyl cellulose. Alternatively, human or murine mCherry- or GFP-labeled breast cancer cells (MDA-MB-231, 4T1—8×10⁴ cells/ml) alone or in combination with iRFP-labeled or unlabeled human astrocytes (16×10⁴ cells/ml—in ratio 1:2) or unlabeled murine astrocytes (8×10⁴ cells/ml—in ratio 1:1) were grown in AM supplemented with 0.24 w/v % methyl cellulose. Cells were seeded in 25 μL droplets on the inner side of a 20 mm dish and incubated for 48 h at 37° C. when the plate is facing upside down to allow for spheroid formation. For basal invasion assay, 3D spheroids were then embedded in GFR Matrigel® and exposed to astrocytes SFM. For cytokines-induced invasion, 3D mono-culture spheroids were grown in astrocytes CM. In order to evaluate the inhibition of invasion following bindarit treatment, WM115 and D4M.3A (4×10¹ cells/ml) alone or in combination with human or murine astrocytes (4×10⁵ cells/ml—in ratio 1:1) were exposed to astrocytes SFM alone or supplemented with 0.35 mM bindarit, or to untreated astrocytes CM or supplemented with 0.35 mM bindarit-treated astrocytes. 3D MCTS invasion was visualized following 24 h and 48 h using EVOS FL Auto cell imaging system (Life Technologies, USA) at 10× magnification. The sprouting of MCTS was analyzed using ImageJ 1.52v software, and the results were presented as total area covered or % of total area covered per spheroid (pixel density) per well.

For breast cancer spheroids including PBMCs, 3D MCTS were prepared by generating a cell suspension consisting of mCherry-labeled MDA-MB-231 (1×10³ cells/well) grown as monocultures or co-cultured with human astrocytes (1×10³ cells/well) and human microglia (1×10² cells/well) in ultra-low attachment U-shaped bottom plates. 100 μL of the cell suspension were seeded in each well, and cells were incubated for 72 h at 37° C. in 5% CO2. After the formation of spheroids, 50 μL of activated PBMCs (1×10⁴ cells/well) were added to the wells and topped with additional 50 μL of medium, with or without 0.3 mM bindarit or 0.5 μM SELPi. The plates were incubated for 7 days at 37° C. in 5% CO2 and cell fluorescence was followed and quantified by Cytation™ C10.

Western Blot. To assess the level of NF-κb pathway activation in the presence of bindarit, human astrocytes were exposed for 2 h to astrocytes SFM supplemented with 0.35 mM bindarit. Astrocytes were then washed and exposed to B16-F10 CM with or without freshly addition of bindarit for 1 h and 30 min. Cells were subsequently lysed in RIPA buffer supplemented with fresh proteases and phosphatases inhibitors (Invitrogen, USA). Cell lysates were analyzed by sodium dodecyl sulfate-polyacrylamide gel electrophoresis (SDS-PAGE), proteins were then transferred on a nitrocellulose membrane and blocked with 5% BSA in Tris-HCl buffer with 1% Tween-20. Anti-phosphorylated p65 (p-p65) and anti-total p65 antibodies were incubated with the nitrocellulose membrane overnight at 4° C. Anti-vinculin antibody was incubated for 1 h at room temperature, and used as loading control. HRP-conjugated anti-rabbit secondary antibody was incubated for 1 h at room temperature. SuperSignal™ West Pico Plus chemiluminescent substrate was added prior to membrane development using iBright 1500 (Life Technologies, USA). Pixel densities of the protein bands were quantified using Image J software.

Flow cytometry. For flow cytometry assays, cells were harvested and washed with FACS buffer. For macrophages in vitro activation, primary murine macrophages (differentiated from BMDC) were treated with RPMI macrophages medium, INF-γ+LPS, rh/mMCP-1, astrocytes CM, astrocytes CM+bindarit, B16-F10 CM, or B16-F10 CM+bindarit for 24 h. Cells were incubated with PE-Vio®770-anti CD11b, FITC-anti F4/80, PE-anti CD206, or APC-Vio®770-anti CD38. For melanoma basal expression of CCR2 and CCR4, B16-F10 cells (5×10⁵ cells/10 cm² dish) were grown in melanoma medium for 24 h. Cells were incubated with APC-anti CCR2 and PE-anti CCR4. For breast cancer basal expression of CCR2 and CCR4, 4T1 cells (5×10⁵ cells/10 cm² dish) were grown in AM or astrocytes CM for 24 h. Cells were incubated with APC-anti CCR2 and PE-anti CCR4 for 1 h on ice. For immune-infiltration assessment, cells from freshly isolated D4M.3A MBM tumors, were divided into two panels: (1) T cell panel-cells were incubated with FITC-labeled anti-CD3, APC-labeled anti-CD8 and VioBlue® anti-CD4 antibodies; (2) myeloid-derived and macrophages panel-cells were incubated with FITC-anti CD11c, APC-Cy7-anti CD11b, APC-anti Gr-1, or FITC-anti F4/80. For immune-infiltration assessment, cells from freshly isolated B16-F10 MBM tumors K/O for CCR2 and CCR4, were divided into two panels: (3) Treg panel-cells were incubated with PerCP-anti CD3, VioBlue® anti-CD4, FITC-anti CD25, and Alexa Fluor®-647 anti-FoxP3 following cell membrane fixation and permeabilization; (4) Macrophages activation panel: cells were incubated with PE-Vio®770-anti CD11b, FITC-anti F4/80, VioBlue® anti-MHC II, PE-anti CD206, APC-anti CCR2. For immune-infiltration assessment, cells from freshly isolated B16-F10 and RET MBM tumors, were divided into two panels: (5) T cell activation panel: cells were incubated with VioBlue®-anti CD3, APC-anti CD8, Alexa Fluor®488-anti CD107. Cells were incubated for 30 min at room temperature. Single stained cells for each antibody and a pool of the corresponding isotype control were used as negative staining controls. Splenocytes isolated from the same animals were used as positive control to discriminate for immune cells population. Cells were then run and analyzed using CytoFlex cytometer and CytoExpert analysis software or Attune NxT cytometer and Kaluza analysis 1.3 software.

CCR2 and CCR4 markers sorting. For basal expression of CCR2 and CCR4 receptors, B16-F10 cells were grown till confluent and harvested using FACS buffer. CCR2-APC and CCR4-PE primary antibodies were incubated in FACS buffer with the cell's suspension for 30 min at room temperature. CCR2+/CCR4+ B16-F10 expressing cells were sorted using ArialII (BD Bioscience, USA) and cultured for a week melanoma medium. The sorted cells underwent CRISPR/Cas9 K/O or NTC K/O and then sorted again for CCR2−/CCR4− following incubation with CCR2-PE and CCR4-APC.

CRISPR/Cas9 for CCR2/CCR4 melanoma B16-F10 knockout (K/O). For B16-F10 CCR2/CCR4 knock-out (K/O), cells were sorted for CCR2+/CCR4+ expression and cultured in plate for a week. Then, electroporation was performed on 1×10⁵ cells/μl with 18.3 pmol Alt-R spCas9 Nuclease V3, and 22 pmol sgRNA, in Buffer R using a Neon electroporation system at 1600v 20 ms 1pulse. CRISPR/Cas9 negative K/O was generated in absence of gRNAs (NTC). Subsequently, cells were grown overnight in a 6-well plate at the concentration of 1×10⁶ cells/ml in antibiotic-free media. K/O assessment was then performed by flow cytometry using Attune NxT on day 3 following electroporation. Finally, B16-F10 cells were sorted for CCR2−/CCR4−

CRISPR/Cas9 sgRNA sequences: CCR2 5′-AGTATGCCGTGGATGAACTG-3′; CCR4 5′-CAGACCCAACAAGAAGACCA-3′ (SEQ ID NO: 10).

CCR2 and CCR4 K/O genes quantification. For quantification of gRNA activity, genomic DNA was extracted using Quick DNA miniprep kit and 500 ng genomic DNA was amplified by PCR using PrimeSTAR MAX for 35cyles. Resulting amplicon was denatured and reannealed in a thermocycler prior to cleaving by T7 Endonuclease 1 at 37° C. for 30 min. DNA cleavage was analyzed by agarose gel electrophoresis and quantified using Biovision (Vilber Lourmat) using a rolling ball for background subtraction. Efficiency of gene editing was calculated as cleavage efficiency (25).

Cleavage efficiency:100×(1−(1−fraction cleaved)^{1/2}.

Evans blue permeability Miles assay. B16-F10 MBM bearing mice were injected i.v. with 0.15 ml of Evans blue solution (30 mg/ml in saline) (N=3 PBS treated-group; N=4 bindarit-treated group). Thirty minutes later, mice were perfused with PBS, brains were harvested, and intracranially tumors were isolated and incubated in 0.5 ml formamide (Sigma-Aldrich, Israel) for 48 h at 55° C. The absorbance of the Evans blue dye was then measured at 620 nm and the blank (formamide only) was subtracted to the Evans blue measurements, using SpectraMax M5 plate reader (Molecular Devices, San Jose, California, USA).

Ethics Statement. All animal procedures were performed in compliance with Tel Aviv University and approved by the Institutional Animal Care and Use Committee (protocol no. 01-16-054, no. 01-21-006, and no. 01-21-004). Post-mortem human brain tissue was obtained by autopsy from the Offices of the Chief Medical Examiner of the District of Columbia, and of the Commonwealth of Virginia, Northern District, all with informed consent from the legal next of kin (protocol 90-M-0142 approved by the NIMH/NIH IRB). Additional post-mortem human brain tissue samples were provided by the National Institute of Child Health and Human Development Brain and Tissue Bank for Developmental Disorders (www(dot)BTBank(dot)org) under contracts NO1-HD-4-3368 and NO1-HD-4-3383. The IRB of the University of Maryland at Baltimore and the State of Maryland approved the protocol, and the tissue was donated to the Lieber Institute for Brain Development under the terms of a Material Transfer Agreement. Clinical characterization, diagnoses, and macro- and microscopic neuropathological examinations were performed on all samples using a standardized paradigm, and subjects with evidence of macro- or microscopic neuropathology were excluded. Details of tissue acquisition, handling, processing, dissection, clinical characterization, diagnoses, neuropathological examinations, RNA extraction, and quality control measures were as described previously (26). The Brain and Tissue Bank cases were handled in a similar fashion (www(dot)medschool(dot)umaryland(dot)edu/BTBank/ProtocolMethods(dot)html).

Animal models: PM tumor. To generate primary melanoma prior MBM, D4M.3A and B2905 cells—0.5×10⁶ cells/100 μl or B16-F10 and Mel-ret—0.1×10⁶ cells/100 μl were inoculated i.d. in immunocompetent C57BL/6 mice. Then, primary lesions were resected once they reached a volume of 75-150 mm³. Whereas, in order to evaluate the sensitivity of PM to immunotherapy (anti-PD-1 treatment), D4M.3A and B2905 cells—0.5×10⁶ cells/100 μl or B16-F10 and Mel-ret-0.1×10⁶ cells/100 μl were inoculated i.d. in immunocompetent C57BL/6 mice. On day 7 once PM reached a volume of 50-75 mm³, they were treated i.p. twice a week with 10 mg/kg anti-PD-1 (Cat. ICH1132; Lot. 1220L520; ichorbio Ltd., Wantage, USA) or isotype control (Cat. ICH2244; Lot. IB4311; ichorbio Ltd., Wantage, USA) antibodies.

Spontaneous MBM tumor. To evaluate activation of astrocytes upon melanoma colonization to the brain, mCherry-labeled 131/4-5B1 cells (1×10⁶ cells/100 μl), WM115 cells (2×10⁵ cells/100 μl), or D4M.3A cells (1×10⁶ cells/100 μl), were injected by ultrasound-guided inoculation to 6-8 weeks old male immunocompromised SCID mice, or immunocompetent C57BL/6 mice. Tumor growth was monitored using an intravital fluorescence imaging system (CRI Maestro™) twice a week for the following 4 weeks post melanoma cell inoculation.

Melanoma Brain Tumors. For drug efficacy in intervention studies, B16-F10 cells (1.5×10⁴ cells/2 μl) were intracranially inoculated stereotactically to the striatum of 8 to 10-week-old C57BL/6 male. Three days following cells inoculation, mice were treated i.v. with 100 mg/kg bindarit or PBS QOD. Mice body weight change was monitored twice a week, and tumor growth was followed using computed tomography (CT), and 4.7T/1H magnetic resonance imaging (MRI, MR Solutions, UK). On day 10 and 13 post melanoma cells inoculation, mice were euthanized and immediately perfused with 4% PFA in PBS. Brains were then harvested for further immunostaining analysis. For functional evaluation of CRISPR/Cas9 K/O engineered cells, B16-F10 CCR2/CCR4 K/O cells or NTC, or WT cells (1.5×10⁴ cells/2 μl) were intracranially inoculated stereotactically into the striatum of 7-week-old male C57BL/6 mice. Mice body weight change was monitored twice a week, and tumor growth was measured at day 9 using 7 T MRI (MR Solutions, UK). Mice were euthanized and brains were then harvested for further immunostaining and flow cytometry analysis. For drug efficacy in a prevention study, 8-week-old male C57BL/6 mice were intradermally (i.d.) injected with D4M.3A, B16-F10, B2905 melanoma cells (5×10⁵ cells/100 μl). Tumor growth was measured twice a week using a caliper, and once it reached a size of 70-150 mm³, tumors were resected. Mice were immediately administered i.v. with 100 mg/kg bindarit or PBS QOD. Two-weeks post primary tumor resection, B16-F10 (1.5×10⁴ cells/2 μl) D4M.3A and B2905 cells (5×10⁴ cells/2 μl) cells were inoculated into the striatum of the same mice (previously bearing the primary tumors). Mice body weight was monitored twice a week, and tumor growth was measured at least two times using 4.7T/1H MRI. On day 8, 12, 16, or 20 mice were euthanized, brains were harvested for further immunostaining and flow cytometry analysis.

In vivo imaging. Tumor bearing mice were imaged at the Sackler Cellular & Molecular Imaging Center (SCMIC), Tel Aviv University. Intravital fluorescence Imaging. CRI Maestro™ non-invasive fluorescence imaging system was used to follow tumor progression of mice bearing mCherry-labeled brain metastasis. Mice were placed inside the imaging system.

Multispectral image-cubes were obtained through 550-800 nm spectral range in 10 nm steps using excitation (575-605 nm) and emission (645 nm longpass) filter set. Mice autofluorescence and background signals were eliminated by spectral analysis and linear un-mixing algorithm.

Magnetic Resonance Imaging (MRI). Tumor bearing mice were scanned using 4.7T/1H MRS 4000™ or 7T MRI (MR Solutions, UK) (32-channel head-coil) following gadolinium i.p. injection. MRI scans were taken using a conventional T1 according to FSE26 sequence. T1 data were analyzed using RadiAnt DICOM Viewer or MRIcro software, and the tumor area was calculated per each axial scan. The tumor volume was obtained by summing the tumor areas from each scan.

Computed Tomography (CT). A CT scan protocol was run by taking multiple X-rays at various angles and then utilizing those X-rays to form a three-dimensional image of the mouse brain. CT data was analyzed using RadiAnt DICOM Viewer software and the tumor volume was obtained by calculating the tumor area from axial and sagittal scans.

Human specimens. FFPE of melanoma, breast cancer and lung cancer samples (primary and brain metastasis), and PBMCs isolated from healthy blood donors, were obtained from Sheba Medical Center following an informed consent. The manipulation of the human samples for immunostaining was accepted by the ethics committees of Tel Aviv University and Sheba Medical Center, under an approved institutional review board (5727-18-SMC). A total of six primary melanomas and brain metastasis pairs were collected. Healthy human brain samples were collected by Thomas Hyde at the Lieber Institute (Baltimore, MD, USA) as described in the manuscript under an approved IRB protocol No. 90-M-0142.

### Immunohistochemistry

Frozen O.C.T. tissue fixation and preparation. Tumor bearing mice were anesthetized using ketamine (100 mg/kg) and xylazine (12 mg/kg) and perfused with 4% PFA in PBS. Mouse brains were resected, incubated with 4% PFA for 4 h followed by 0.5 M of D-Sucrose for 1 h, and 1 μM D-Sucrose overnight. Tissues were then embedded in O.C.T. on dry ice and stored at −80° C.

Tissues Staining. For immunostaining of brain tumors, frozen O.C.T.-embedded tissues, or FFPE tissues, were cryo-sectioned into 5 μm thick sections. O.C.T Slides were fixed and permeabilized in Acetone for 20 min at room temperature. Briefly, slides were incubated with goat serum (10% goat serum in PBS×1+0.02% Tween-20) for 30 min to block non-specific binding sites. Slides were stained for morphology by hematoxylin and eosin (H&E), or immunostained for: anti-mouse/human MCP-1, anti-mouse/human GFAP, anti-mouse/human CCR2, anti-mouse/human CCR4, anti-mouse/human Iba1, anti-mouse CD31, anti-mouse/human Ki67, anti-mouse CD8a, anti-mouse F4/80 anti-mouse IL-6, anti-mouse/human PD-1, anti-mouse PD-L1, anti-mouse CD206, anti-human SELP, and anti-human/mouse PSGL-1. After 1 h incubation, slides were incubated with secondary antibodies for an additional 1 h: goat anti-rabbit Alexa Fluor® 488 for GFAP, CCR2 and Ki67; goat anti-mouse Alexa Fluor®647 for MCP-1, NK-1.1, and CD206 markers; goat anti-rabbit Alexa Fluor®647 for PD-1, CCR4, and Ki67 markers; goat anti-rat Fluor® 488 for CD31, CD8a, F4/80, IL-6, or PD-L1, markers. Nuclei were counterstained using Hoechst solution. The stained tissues were then fixed and mounted on a glass microscope slide using ProLong™ Gold antifade mounting. Fluorescence and brightfield images were captured using a fluorescence and brightfield illumination microscope (Evos FL Auto, life technologies) at 40× and 10× magnifications, respectively.

Data and software availability. Expression profile of CCR2/4 (y-axis) in 55 melanoma cell lines derived from various sites (3 PM, 2 MBM, 2 lung metastasis, 12 lymph node metastasis, 1 axillary node metastasis, 1 pelvic wall metastasis, 1 pleural metastasis, 1 soft tissue and 1 subcutaneous tissue metastasis and 30 from unknown origin) (x-axis) from CCLE dataset. Median and P value (two-sided) computed using Wilcoxon rank-sum test.

Statistical Methods. Data is expressed as mean±standard deviation (S.D.) for in vitro assays and as mean±standard error of the mean (S.E.M.) for in vivo assays. Statistical analyses were performed with Student's t-test or one-way ANOVA unless noted otherwise. Statistical significance in mice OS was determined by the log-rank test using GraphPad Prism software (GraphPad Software Inc.). The P values under P<0.05 were considered statistically significant.

Multicellular tumor spheroids (MCTS). 3D tumor spheroids were formed from a mixture of melanoma and astrocytes. Human or murine mCherry-labeled melanoma cells (131/4-5B1 and D4M.3A—4×10⁵ cells/ml) in combination with human unlabeled murine astrocytes (4×10⁵ cells/ml—in ratio 1:1), human or murine endothelial cells (hBECs in green or mBECs unlabeled (8×10⁵ cells/ml—in ratio 2:1)) and unlabeled microglia ((1×10⁵ cells/ml—in ratio 0.25:1), were grown in AM supplemented with 0.24 w/v % methyl cellulose. Cells were seeded in 25 μL droplets on the inner side of a 20 mm dish and incubated for 48 h at 37° C. when the plate is facing upside down to allow for spheroid formation. In order to evaluate the inhibition of invasion following BRAF and MEK inhibitors in combination with bindarit treatment, 3D MCTS were exposed to astrocytes SFM alone or supplemented with 3 μM polyglutamic acid-Cy5 (PGA), 3 μM SLM-DBF-diol BRAF and MEK inhibitors (BRAFi and MEKi), PGA-Cy5 3 μM SLM-DBF, 500 M bindarit, 3 μM SLM-DBF-diol+500 μM bindarit, PGA-Cy5 3 μM SLM-DBF+500 μM bindarit, or with 1.75 μM poly (lactic-co-glycolic acid, PLGA) NP (empty NP), 1.75 μM DBF (inhibitors (BRAFi), 1.75 μM DBF-NP, 500 μM bindarit, 1.75 μM DBF-NP+500 μM bindarit, 1.75 μM DBF NP+500 μM bindarit. 3D MCTS invasion was visualized following 48 h using EVOS FL Auto cell imaging system (Life Technologies, USA) at 10× magnification.

Synthesis of nanovaccine. PLGA/PLA MART-1 nanovaccine was formulated as previously describe (19).

Flow cytometry for immune cells infiltration following nanovaccine and bindarit combination treatment. To assess the infiltration of immune cells into B16-F10 brain tumors following treatment with nanovaccine, bindarit or the combination, tumors were resected at day 18. Cells were isolated and washed using PBS buffer supplemented with 2 mM EDTA and 0.5% BSA, and 0.1% sodium azide (FACS buffer) and incubated with anti-CD3-Vioblue, anti-CD8-APC, or anti-CD4-Vioblue, anti-CD25-FITC, anti-FOxP3-Alexa647, or anti-CD11c-FITC, anti-MHC-II-Pacific blue, or anti-CD103-PE, anti-CD11c-FITC, anti-MHC-II-Pacific blue, or anti-CD44-FITC, anti-CD3-Vioblue, anti-CD8-APC for 30 min at room temperature. Each of the immune populations were then run and analyzed using Attune NxT cytometer and Kaluza analysis 1.3 software.

Animal model to test nanovaccine and bindarit treatment efficacy. To generate primary melanoma prior MBM, B16-F10—0.1×10⁶ cells/100 μl were inoculated i.d. in immunocompetent C57BL/6 mice. Then, primary lesions were resected once they reached a volume of 100-150 mm³. Ten days post resection of the primary tumor, mice were randomized and injected i.v. with one dose of bindarit (3 mg/kg) every other day (QOD) until day 16 and intranasal administration of nano-vaccine containing MART-1 peptides (4.5 mg/kg) or PBS ones a week for three weeks, alone or in combination. After primary tumor resection, B16-F10 melanoma cells (15.000 cells) were injected intracranially and mice where continuous treated.

Cell cycle assay. MART-1⁺ melanoma cell cycle arrest was assessed followed isolation of melanoma cells from the brain at day 18. Cells were isolated and fix in 70% ethanol for 30 minutes at 4° C. Cells were then washed using PBS buffer supplemented with 2 mM EDTA and incubated with 1 μg/mL propidium iodine and immediately run and analyzed using Attune NxT cytometer and Kaluza analysis 1.3 software.

Prediction of immune-checkpoint treatment efficacy in MCTS. Following the last immunization with bindarit and nanovaccine or PBS, splenocytes from animal treated with bindarit and PBS were then isolated and activated with with α-CD28 (1 μg/mL) and β-mercapto (50 mM) (plain activation), whereas splenocytes from animal treated with bindarit and nanovaccine were additionally exposed to MHCI/II MART-1 peptides (1 mg/mL) (nanovaccine activation) for a week. Then, 3D MCTS of melanoma B16-F10 cells and astrocytes (4×10⁵ cells/ml—1:1 ratio) were co-cultured with a splenocytes (250×10⁵ cells/ml—1:50 ratio (plain activation or nano-vaccine activation) and treated with immuno-checkpoint inhibitors (α-PD1 or α-OX40—0.1 mg/mL) alone or in combination with bindarit (0.5 mM). 3D MCTS invasion was visualized following 48 h using EVOS FL Auto cell imaging system (Life Technologies, USA) at 10× magnification. The sprouting of MCTS was analyzed using ImageJ 1.52v software, and the results were presented as total area covered or % of total area covered per spheroid (pixel density) per well.

Breast cancer proliferation in co-culture. Human and murine breast cancer mCherry-labeled cells (MDA-MB-231—30×10³ cells/well, 4T1—15×10³ cells/well) were plated alone onto 96-well culture plates in AM. In addition, breast cancer cells were co-cultured with human or murine astrocytes at ratios of 1:1-1:5 in AM. For treatment with bindarit, 0.3 mM bindarit solution was added to the wells.

Breast Cancer Brain Metastases (BCBM). To generate primary breast rumors prior to BCBM, 4T1 cells (3×10⁵ cells/100 μl) were inoculated orthotopically into the mammary fat pad of immunocompetent BALB/C mice. Seven days later, primary lesions were resected. One week post primary tumor resection, 4T1 cells (2×10⁴ cells/2 μl) were intracranially inoculated stereotactically to the striatum of the same mice (previously bearing the primary tumors). Three days following cells inoculation, mice were treated i.v. QOD with 100 mg/kg bindarit (CAS: 130641-38-2; Lot. AGN22-876807-1; Angene Chemical, United Kingdom) or PBS, i.p. twice a week with 10 mg/kg anti-PD-1 (Cat. ICH1132; Lot. 1122L335; ichorbio Ltd., Wantage, UK) or PBS, and i.v. QOD with 16 mg/kg SELPi KF38789 (Cat. 2748; Lot. 2A/276275; Tocris BioScience, Bristol, UK) or vehicle (0.01% DMSO, 93.2 mg/ml polyethyleneglycol and 14.8 mg/ml Tween-80 in DDW). Mice body weight change was monitored three times a week, and tumor growth was followed using 4.7T/1H magnetic resonance imaging (MRI, MR Solutions, UK). PBMCs activation. For human PBMCs activation a 6-well plate was coated with anti-CD3F (Cat. 100340; Lot. B302116; Clone 145-2C11) antibody and 2 μg/ml anti-CD28 (Cat. 102116; Lot. B331922; Clone 3751), and PBMCs were incubated with 10 U/ml rhIL-2 for 2 days.

Gene expression of breast cancer patients. Gene expression of GSE12276 database (Gene Expression Omnibus database) www(dot)ncbi(dot)nlm(dot)nih(dot)gov/geo/in which the RNA expression levels of CCL2, CCR2, CCR4, SELP, SELPLG, CD274, and PDCD1 were compared between primary breast cancer tumors that metastasized to the brain compared to that metastasize to other distant organs. One-tailed non-paired Student's t-test statistical analysis was used.

### Example 1

### Activated Astrocytes Sustain Melanoma Cell Growth, Invasion and Migration

The first aim was to elucidate the mechanism by which activated-astrocytes contribute to MBM progression. Although several studies pointed out potential factors involved in promoting the interaction between astrocytes and melanoma cells, the benefit of their inhibition in terms of clinical relevance is yet to be fully elucidated (10, 27). Previous publications have shown the contribution of astrocytes-secreted cytokines to melanoma cell proliferation and migration in Mel-ret murine melanoma model, as well as WM983BA, 375P, DM-4 (from lymph node metastasis specimens), YDFR.CB3, and TXM-13 human melanoma, in addition to the WM4265-2 human melanoma brain metastatic variant (10, 28-30). Here, the observation was strengthened by using alternative murine (D4M.3A, B2905, B16F10) and human (131/4-5B1, A375, WM115) melanoma models and clinical samples that present different origins, mutations (BRAF, PTEN, etc.), and responsiveness to immunotherapy. Positive staining was shown for GFAP-activated astrocytes surrounding melanoma metastases in a cohort of 40 patients diagnosed with MBM bearing wild-type BRAF (patient-derived PD1-PD5, not shown). This activation was also confirmed by histological analysis of the MBM mouse models of human and murine melanoma bearing different BRAF mutations (intracardially-inoculated mCherry-labeled WM115 (BRAF V600D), D4M.3A (BRAF V600E), or 131/4-5B1 (BRAF V600D) melanoma cells) (not shown). GFAP-activated astrocytes were also observed in healthy human and murine brain samples, but to a much lower extent under physiological conditions (not shown) (31). Following intracardiac cell inoculation of melanoma cells (B2905, D4M.3A, WM115 and 131/4-5B1), different models showed variability in MBM formation and location, as well as in the incidence of metastasis to other organs (Table 4 and 5).

TABLE 4
Incidence of brain metastasis reported in the literature (131/4-5B1, A375, Mel-ret cell
lines) or observed in the study (¥) (B2905, D4M.3A, and 131/4-5B1 cell lines) within 6 weeks
following systemic cell inoculation.

| | Systemic cell inoculation | | Metastasis site | | |
|---|---|---|---|---|---|
| Melanoma | | Primary | Leptomeningeal | Parenchymal | |
| cells | Intracardiac | resection | MBM | MBM | Other sites |
| 131/4-5B1 | 70% | 54% | Occasional (32) | Predominantly | Thorax cavity (0%); |
| | (N = 28/40) | (N = 4/6) | | (32) | Lungs (0%); Liver |
| | (32) | (21) | | | (2.5%); Adrenals |
| | 100% | | | | (52.5%); Mesentery |
| | (N = 10/10)^{¥} | | | | (0%); Ovaries (35%); |
| | | | | | others (0%) (32) |
| | | | | | Lungs (N = 80%); Kidney |
| | | | | | (N = 40%) (21) |
| | | | | | Lungs (10%)^{¥} |
| A375 | 60% | | Not shown (33) | Predominantly | Not mentioned (33) |
| | (N = 6/10) | | | (33) | |
| | (33) | | | | |
| WM115 | 0% (N = 0/10)^{¥}* | | | | Not found^{¥} |
| B16-F10 | 27.5% | | Predominantly | Occasional | Thorax cavity (22.5%); |
| | (N = 11/40) | | (32) | (32) | Lungs (32.5%); Liver |
| | (32) | | | | (22.5%); Adrenals |
| | | | | | (60%); Mesentery |
| | | | | | (32.5%); Ovaries (80%); |
| | | | | | others (7-17%) (32) |
| D4M.3A | N = 63% | | Yes + | Yes^{¥} | Thorax cavity (88%); |
| | (N = 7/9)^{¥} | | extracranial | | Heart (44%); Lungs |
| | | | metastasis^{¥} | | (22%); Liver (44%); |
| | | | | | Adrenals (66%); |
| | | | | | Pancreas (66%); |
| | | | | | Peritoneal cavity and |
| | | | | | intestine (44-88%)^{¥} |
| Mel-ret | 50% | 23% | Not shown (29) | Predominantly | Lungs (50%) (29) |
| | (N = 6/12) | (N = 14/60) | | (29) | |
| | (29) | (29) | | | |
| B2905 | 90% | | Not found^{¥} | Yes^{¥} | Thorax cavity (60%); |
| | N = (9/10)^{¥} | | | | Heart (90%); Lungs |
| | | | | | (50%); Liver (40%); |
| | | | | | Adrenals (0%); Testicles |
| | | | | | (40%); others (10-40%)^{¥} |

*66% (N = 4/6) 2-3 months following intracardiac cell injection

TABLE 5
Incidence of MBM and metastasis to distant organs observed in the study (B2905,
D4M.3A, and 131/4-5B1 cell lines) following intracardiac systemic cell inoculation.

| | | | | | Testis/Gonadal | | | | | |
|---|---|---|---|---|---|---|---|---|---|---|
| | Thorax | Lungs | Heart | Liver | adipose tissue | Gut | Pancreas | Kidney | Brain | Peritoneum |
| B2905 | | | | | | | | | | |
| cells | | | | | | | | | | |
| 1-1 | 1 | 1 | 1 | 1 | | | | | | |
| 1-2 | | | 1 | 1 | | | | | 1 | |
| 1-3 | 1 | | 1 | | | | | | 1 | |
| 1-4 | 1 | | 1 | | 1 | 1 | 1 | | 1 | |
| 1-5 | 1 | 1 | | | | 1 | 1 | | 1 | |
| 2-1 | | | 1 | 1 | | | 1 | | 1 | |
| 2-2 | 1 | 1 | 1 | 1 | 1 | | | | 1 | 1 |
| 2-3 | | 1 | 1 | | 1 | | | | 1 | |
| 2-4 | 1 | 1 | 1 | | | | | | 1 | |
| 2-5 | | | 1 | 1 | 1 | | 1 | | 1 | |
| n = 10 | 6/10 | 5/10 | 9/10 | 4/10 | 4/10 | 2/10 | 4/10 | 0/10 | 9/10 | 1/10 |
| D4M.3A | | | | | | | | | | |
| cells | | | | | | | | | | |
| 1-1 | 1 | | 1 | | | | 1 | 1 | 1 | 1 |
| 1-2 | 1 | | | | | | | | 1 | |
| 1-3 | 1 | | | | | 1 | 1 | 1 | 1 + | 1 |
| | | | | | | | | | extract. | |
| 1-4 | 1 | 1 | 1 | 1 | | 1 | | 1 | 1 + | 1 |
| | | | | | | | | | extract | |
| 1-5 | 1 | | | | | | 1 | 1 | | 1 |
| 2-1 | | | 1 | 1 | | 1 | 1 | | extract | 1 |
| 2-2 | 1 | | | | | 1 | 1 | | 1 + | 1 |
| | | | | | | | | | extract | |
| 2-3 | 1 | 1 | 1 | 1 | 1 | 1 | 1 | 1 | 1 | 1 |
| 2-4 | 1 | | | 1 | | 1 | | 1 | 1 | 1 |
| n = 9 | 8/9 | 2/9 | 4/9 | 4/9 | 1/9 | 6/9 | 6/9 | 6/9 | 7/9 | 8/9 |
| 131/4-5B1 | | | | | | | | | | |
| cells | | | | | | | | | | |
| 1-1 | | | | | | | | | 1 | |
| 1-2 | | | | | | | | | 1 | |
| 1-3 | | | | | | | | | 1 | |
| 1-4 | | 1 | | | | | | | 1 | |
| 1-5 | | | | | | | | | 1 | |
| 2-1 | | | | | | | | | 1 | |
| 2-2 | | | | | | | | | 1 | |
| 2-3 | | | | | | | | | 1 | |
| 2-4 | | | | | | | | | 1 | |
| 2-5 | | | | | | | | | 1 | |
| n = 10 | 0/10 | 1/10 | 0/10 | 0/10 | 0/10 | 0/10 | 0/10 | 0/10 | 10/10 | 0/10 |

Presence of metastasis = 1; extract = presence of extracranial MBM.

Thus, in order to study melanoma-astrocytes interactions in a more reproducible model of brain metastasis only, intracranial cell inoculation following the excision of the primary melanoma lesion was included in the study. The intracranial inoculation per se induces local neuroinflammation (34), mostly affecting resident cells (e.g. astrocytes and microglia), and possibly interfere with their activation and interaction with the cancer cells in the initial steps of brain colonization (35). To subtract the secondary effects associated with the intracranial injection, cells were stained for GFAP-activation brain cryo-sections following sham injury or PBS administration. Then, the morphology and GFAP expression of astrocytes of these control samples were compared to that of astrocytes in B16-F10 and D4M.3A MBM samples (not shown). Only basal activation was detected in the samples with the sham injury after three days. Intermediate GFAP-activation and presence of isomorphic astrocytes were found at the injection site of PBS group (not shown). Indeed, the short-term (day 3) expression indicates physiological brain functionality as it decreased by day 10 (not shown) (36). Nonetheless, high level of reactive astrocytes was observed only in tumor-bearing brain tissue from mice, 10 days following melanoma cell inoculation (not shown).

These findings support the feasibility of using intracranially-injected model of MBM for investigations involving the late contribution of GFAP-expressing astrocytes in MBM progression.

The direct effect of GFAP-activated astrocytes in promoting melanoma cell proliferation and invasion in co-culture were further investigated. To study these effects, astrocytes were co-cultured with mCherry-labeled human or murine melanoma cells (WM115, A375, D4M.3A, B16-F10, B2905 cells). The results show that astrocytes facilitate melanoma cell proliferation by 2-4-fold compared to melanoma cells grown in mono-culture (not shown). Melanoma cell invasion capacity was evaluated in 3D multicellular spheroids composed of melanoma cells grown in mono-culture or in co-culture with astrocytes. It was found that the sprouting capability of mCherry-labeled melanoma cells was significantly enhanced (25% in WM115, 75% in D4M.3A, and 55% in B2905 cells) following co-culture with astrocytes (FIG. 1A). In some cases (A375 and B16-F10 cells), the addition of astrocytes to the mono-cultures of melanoma cells was essential for the formation of 3D multicellular spheroids (not shown).

It was speculated that the first step of melanoma brain colonization and spreading within the brain parenchyma is mediated by activated-astrocytes and followed by cancer cell-astrocytes interactions. Therefore, the effect of stress-induced activated astrocytes (37) conditioned-media (CM) containing growth factors and cytokines secreted by astrocytes was evaluated. It was found that the melanoma cells migrated faster towards the astrocytes CM compared to serum-free medium (SFM) in a Transwell® migration assay (3-, 1.2-, 4-, 1.6-, 1.8-, and 2-fold increase in WM115, A375, D4M.3A, B16-F10, B2905, and 131/4-5B1 cells, respectively, FIG. 1B and not shown).

These results suggest that cytokines or growth factors secreted by astrocytes can interact and promote melanoma cell motility prior to their direct interactions.

### Example 2

### MCP-1 Plays a Key Role in Melanoma Chemo-Attraction Towards Astrocytes CM

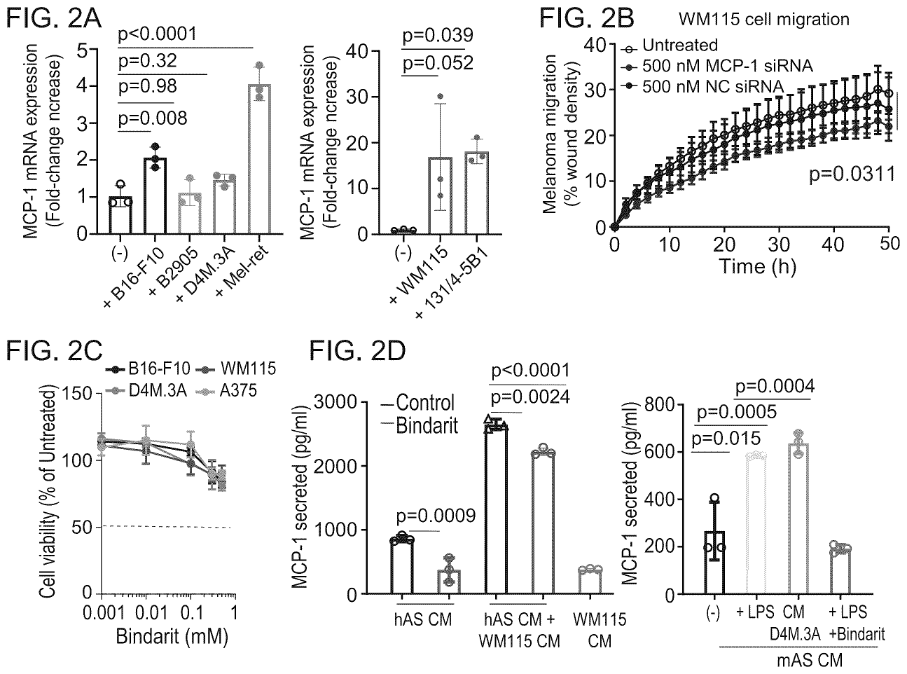

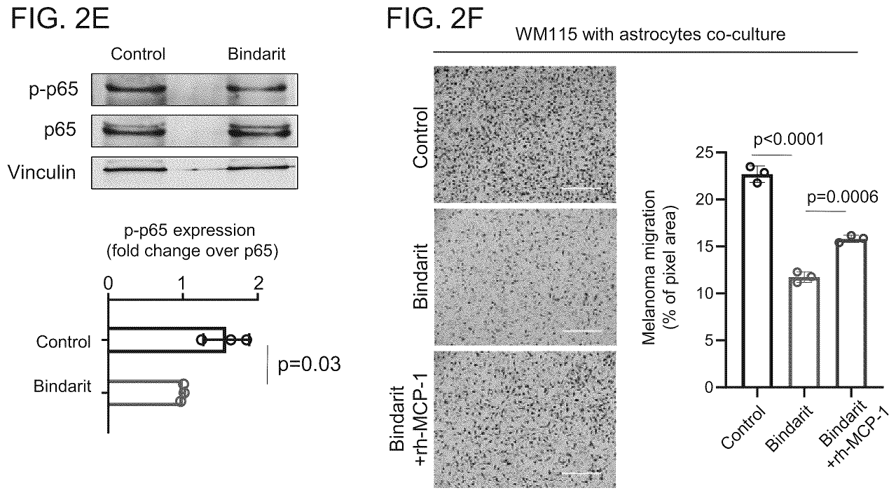

To identify changes in astrocyte secretome upon paracrine interactions with melanoma cells, a cytokine array using melanoma-activated astrocytes CM was performed. The secretion levels of 6 out of 100 cytokines involved in tissue damage response and cell chemotaxis (i.e. MCP-1/CCL2, RANTES/CCL5, GRO-α/CXCL1, and SERPIN E1/PAI, IL-6 and IL-8/MIP-2), were upregulated (2, 2.1, 1.7, 1.1, 1.4, and 1.6 average-fold increase, respectively) in astrocytes grown in CM of several melanoma cell lines (B16-F10, A375, 131/4-5B1, WM115, and D4M.3A) (FIG. 1C, not shown). Following the inhibition of the activity of the selected cytokines by adding neutralizing antibodies to the astrocytes CM, both murine D4M.3A and human WM115 melanoma cell Transwell® migration was reduced compared to cells grown in control astrocytes CM (FIG. 1D). The strongest and most statistically significant migration inhibition was achieved with anti-MCP-1 (33% in D4M.3A and 40% in WM115 cells) and anti-IL-8/MIP2 (45% in D4M.3A and 35% in WM115 cells) antibodies. Indeed, MCP-1 was found to be a potent factor for melanoma migration in all the cell lines tested, including human primary and metastatic melanoma (WM115 and 151/4-5B1) and murine primary melanoma cell lines (D4M.3A and B16-F10)—40% and 50% migration inhibition (FIG. 1D and not shown). Recombinant human MCP-1 (rh-MCP-1) enhanced melanoma cell migration in a wound healing assay (5% increase in WM115, 27% increase in 131/4-5B1), whereas MCP-1 neutralizing antibody reduced wound closure (7% reduction in WM115, 66% reduction in 131/5B1) compared to cells that migrated in astrocytes SFM or astrocytes CM (not shown). Nevertheless, co-culturing astrocytes with B16-F10, B2905, D4M.3A and Mel-ret murine melanoma cells or WM115 and 131/4-5B1 human melanoma cells led to 2.1, 1.1, 1.5 and 4-fold increase or 15-fold and 15-fold increase, respectively, in their MCP-1 mRNA expression compared to astrocytes grown as mono-culture (FIG. 2A).

To strictly address the importance of astrocyte-secreted MCP-1 in attracting melanoma cells and promoting their growth, the ability of melanoma cells to migrate in co-culture towards astrocytes, microglia, or endothelial cells (known to be the alternative sources of MCP-1 in the brain) was compared, following neutralization of MCP-1 secretion. It was observed that the neutralization of MCP-1 reduced melanoma cell migration towards the relevant brain resident cells with a statistically significant difference towards microglia (p=0.024) and astrocytes (p=0.052) but not towards endothelial cells (not shown). MCP-1 neutralization did not affect the proliferation of melanoma cells when co-cultured with microglia, but it did inhibit their proliferation when co-cultured with astrocytes or endothelial cells (not shown). It can be concluded that the neutralization of MCP-1 in CM of astrocytes-melanoma cells co-culture inhibited melanoma cell migration and proliferation, and it did not affect the viability of the brain resident cells (not shown). Astrocytes, microglia and endothelial cells express and secrete MCP-1, as it is demonstrated by its expression and secretion in their intracellular compartments and in their conditioned media when stimulated in SFM, or when co-cultured with WM115 melanoma cells (not shown). However, when in co-culture with melanoma cells, astrocytes were shown to express and secrete MCP-1 at a higher extent compared to microglia and endothelial cells (not shown).

To inhibit MCP-1 mRNA, a small interfering RNA (siRNA) was used to knockdown its expression in human astrocytes. Astrocytes treated with siRNA:PEI polyplex expressed less than 20% of the total MCP-1 mRNA compared to untreated or negative control (NC) siRNA-treated cells (not shown). The siRNA:PEI polyplexes did not cause any off-target effects on astrocytes viability (not shown). Moreover, ELISA quantification of astrocytes CM following treatment with siRNA:PEI polyplex showed a remarkable reduction in MCP-1-secreted chemokine (80% of the untreated) (not shown). The siRNA-treated astrocytes-derived CM added to WM115 or 131/4-5B1 melanoma cells led to 20 to 38% decrease of the migration ability of melanoma cells compared to the same cells grown in untreated astrocytes CM or astrocytes treated with negative control siRNA (NC:PEI) (FIG. 2B). The neutralization or silencing of MCP-1 resulted in a decrease of 131/4-5B1 melanoma cell migration (not shown). This effect was more pronounced in the 131/4-5B1 brain metastasis cell line than in the WM115 melanoma cell line (FIG. 2B and not shown).

### Example 3

### The MCP-1 Inhibitor Bindarit Inhibits Melanoma Cell Migration and Invasion in Astrocytes-Melanoma Cells Co-Culture

The establishment of astrocytes-melanoma cells interactions in which MCP-1, overexpressed by astrocytes, enhances in vitro melanoma tumorigenic properties (proliferation, migration and invasion), drove the research to further evaluate the effect of inhibiting MCP-1 via bindarit. Bindarit did not affect the viability of neither human nor murine melanoma cells, at all concentrations used when grown as mono-culture (FIG. 2C). As resident brain cells secrete basal levels of MCP-1, the present inventors set to verify that bindarit will not be toxic to these cells. To that end, it is shown that treatment with bindarit did not affect the viability of human brain resident cells (astrocytes, microglia, endothelial cells and human hippocampal neurons) (not shown). Moreover, the ability of these brain resident cells to migrate was not inhibited by the inhibition of MCP-1 (neutralizing antibody or bindarit at 0.3 mM, not shown) meaning that MCP-1 inhibition does not affect the viability nor the motility of normal brain resident cells. Bindarit efficiently inhibited MCP-1 secretion from melanoma- or SFM-activated human astrocytes, and in LPS-stimulated murine astrocytes by 0.3- or 3- and 4-fold change, respectively, whereas CM of melanoma cells (WM115 and D4M.3A) enhanced the basal secretion of MCP-1 by 3-fold increase (FIG. 2D). MCP-1 secreted to the CM of bindarit-treated astrocytes was 50% of that of untreated astrocytes, whereas minimal or no effects were observed for the other targets previously found upregulated in the cytokine arrays of activated astrocytes (not shown). Indeed, bindarit, a selective regulator of the classical NF-κB inflammatory pathway, acts as an inhibitor of MCP-1 promoter activation by blocking the phosphorylation of IκBα and p65, a downstream effector of NF-κB signaling pathway, and halting the production of MCP-1 (38). To that end, the selective inhibition of MCP-1 was evaluated via the downregulation of p65 phosphorylation in astrocytes activated by melanoma CM supplemented with bindarit. Phosphorylation of p65 was induced by exposing astrocytes to WM115 CM, while its inhibition was observed in these stimulated astrocytes when exposed to melanoma WM115 CM followed by the treatment with bindarit (FIG. 2E).

The inhibition of MCP-1 production resulted in decreased melanoma cell migration (D4M.3A, B16-F10, A375, 131/4-5B1 and WM115) towards astrocytes, when the latter were exposed to bindarit (not shown). Moreover, the addition of rh-MCP-1 rescued the inhibition of melanoma cell migration as opposed to those cells that migrated in the presence of bindarit-treated astrocytes (p=0.006) or towards the CM of bindarit-treated astrocytes (p=0.115) (FIG. 2F and not shown). The CM of bindarit-treated astrocytes decreased melanoma cell motility, whereas minor effects on melanoma cell migration were observed when melanoma cells were migrating towards astrocytes CM of pre-treated astrocytes (not shown). When the present inventors challenged the migration/invasion of melanoma cells by seeding brain endothelial cells (mBEC) on the Transwell® upper chamber, the neutralization of MCP-1 in astrocytes pre-treated with bindarit, inhibited nearly 15% in D4M.3A and 50% in B16-F10 melanoma trans-endothelial cell migration (not shown). When astrocytes were directly treated while in co-culture with melanoma cells, 50% or inhibition of D4M.3A and B16-F10 trans-endothelial cell migration was achieved (not shown). Moreover, the invasion ability of bindarit-treated mCherry-labeled melanoma multicellular 3D spheroids in Matrigel® (WM115 cells or D4M.3A) was significantly inhibited when co-cultured with astrocytes compared to untreated 3D co-culture (not shown). In the absence of astrocytes, the sprouting ability of melanoma cells was not affected by bindarit, either when melanoma multicellular 3D spheroids were grown in astrocytes SFM or in astrocytes CM (not shown). Thus, even though the migration and invasion abilities of melanoma cells are enhanced in the presence of astrocytes or of alternative brain-resident cells, this phenotype can be altered by blocking MCP-1 secretion from astrocytes.

### Example 4

### Treatment with Bindarit Delays Tumor Progression in Mice Bearing B16-F10 Intracranial MBM

The use of spontaneous (syngeneic) mouse model is limited to highly variable incidence of brain metastasis (50% from direct systemic inoculation of non-tropic melanoma cells, less than 20% from orthotopic inoculation following primary tumor resection (35)) and may be affected by the simultaneous development of metastasis to other distant organs (Table 2).

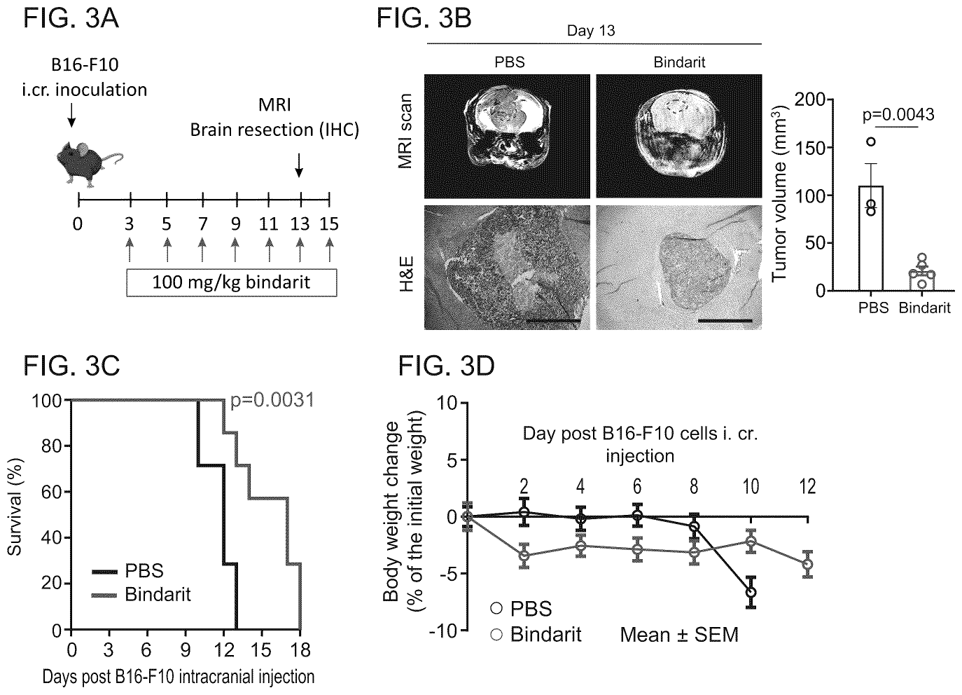

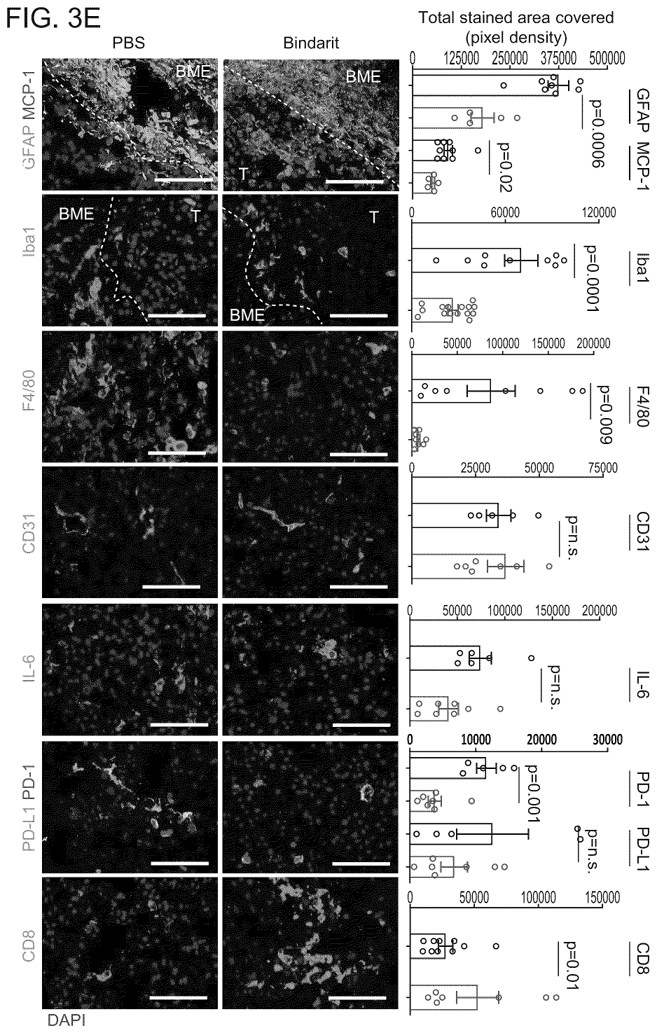

Therefore, intracranial models were used for interventional study as a strategy to reduce variability in number, location, and yield of brain metastases among the treated and control groups. In order to interfere with melanoma-astrocytes and BME interactions via MCP-1 inhibition, in vivo therapeutic studies were carried out by intravenous (i.v.) administration of bindarit (100 mg/kg in PBS) every other day (QOD) in the highly aggressive B16-F10 intracranially-injected melanoma-bearing mice (FIG. 3A). At the dose and treatment regimen selected for the in vivo studies, bindarit did not cause red blood cells (RBC) lysis (not shown). On day 13, magnetic resonance imaging (MRI) analysis showed a remarkable inhibition of tumor growth following treatment with bindarit (FIG. 3B). Inhibition of MCP-1 resulted in 5-fold reduction in tumor size compared to the PBS-treated group (FIG. 3B), and a discrete, although significant, prolongation of mice survival was obtained (FIG. 3C). In addition, no notable body weight change was observed (FIG. 3D). Cryosections of brain samples from day 13, analyzed for tumor morphology (H&E), emphasized the efficacy of the treatment in delaying melanoma progression (FIG. 3B). Further histological analysis showed lower levels of MCP-1 secretion from activated-astrocytes, moderate reduction in F4/80⁺ and Iba1⁺ microglia/macrophages, and enhanced infiltration of CD8⁺ T cells into tumors resected from mice treated with bindarit (FIG. 3E). As MCP-1 is a potent inhibitor of T cell trafficking, the present inventors evaluated the expression of immune inhibitory molecules, such as PD-1/PD-L1, in the TME of bindarit-treated MBM. Reduced expression of co-inhibitory PD-1 molecule was observed in the TME, whereas PD-L1 expression was not altered and no correlation with CD8⁺ T-cell infiltration was confirmed (39). Moreover, quantification of microvessel density (CD31 marker) did not point out any significant changes between PBS and bindarit-treated groups, even though reduction in lumen size of blood vessels was qualitatively seen (FIG. 3E). To evaluate if the reduction in lumen size of blood vessels found in bindarit-treated MBM may be less permeable due to the inhibition of MCP-1, vessel permeability within the tumor was investigated using a modified Miles assay (40). Mice bearing intracranially-inoculated B16-F10 melanoma were treated according to the scheme of treatment presented in FIG. 3A. On day 11, besides achieving inhibition of tumor growth (not shown), mice treated with bindarit treatment resulted in 50% reduction of Evans blue dye extravasation and extraction from the brain parenchyma (not shown). Moreover, early time point (day 7 and 8) treated-tumors of matched size, showed reduction of dye extravasation into the brain tissue at 45′ and 3 h following bindarit administration (not shown).

### Example 5

### MCP-1 Inhibition in MBM Impairs Myeloid-Derived Suppressor Cell Infiltration and Increases Anti-Tumor T-Cell Recruitment

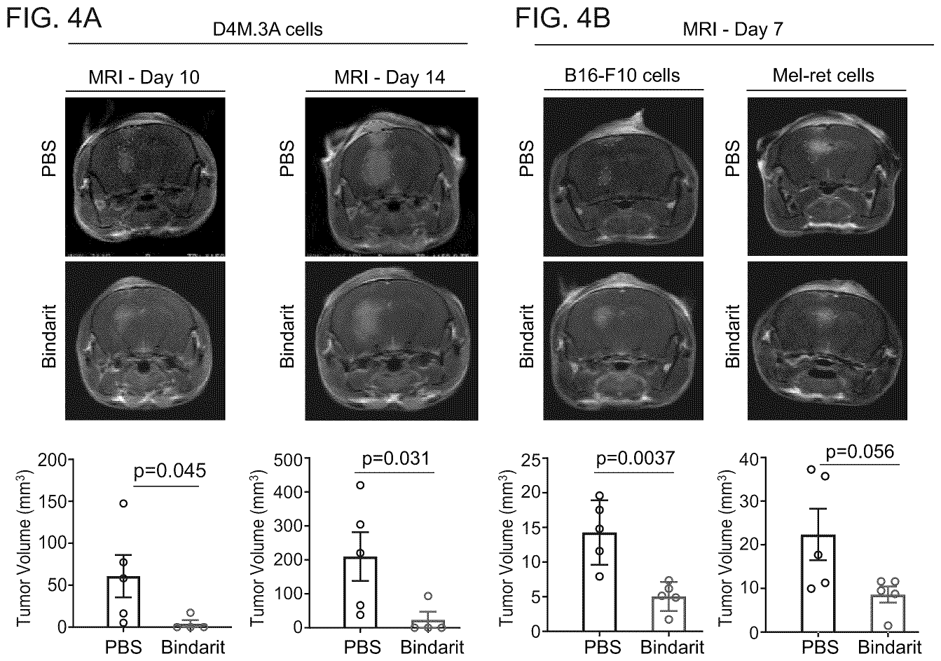

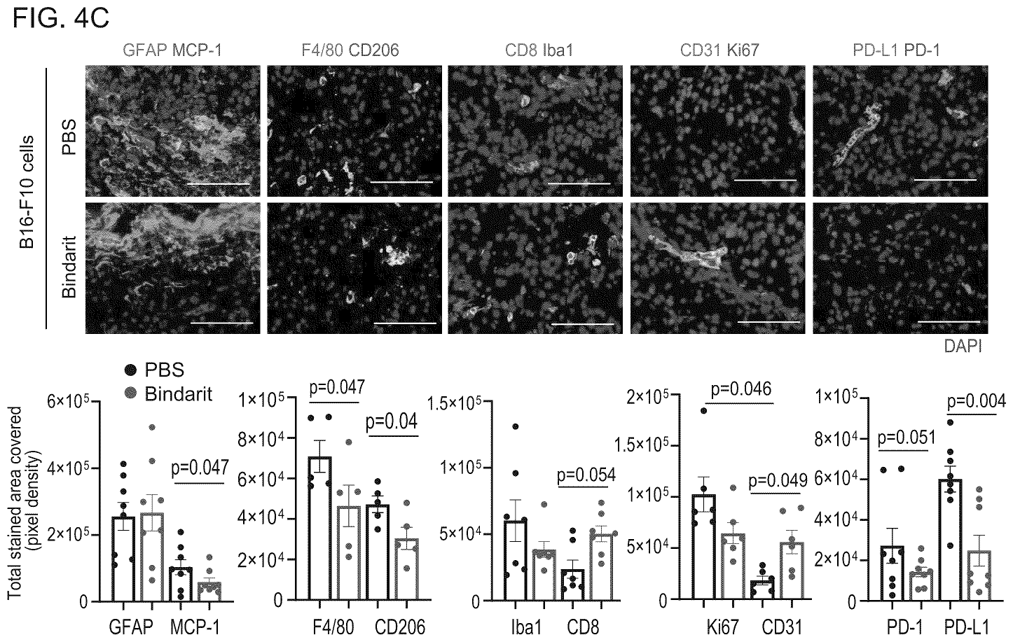

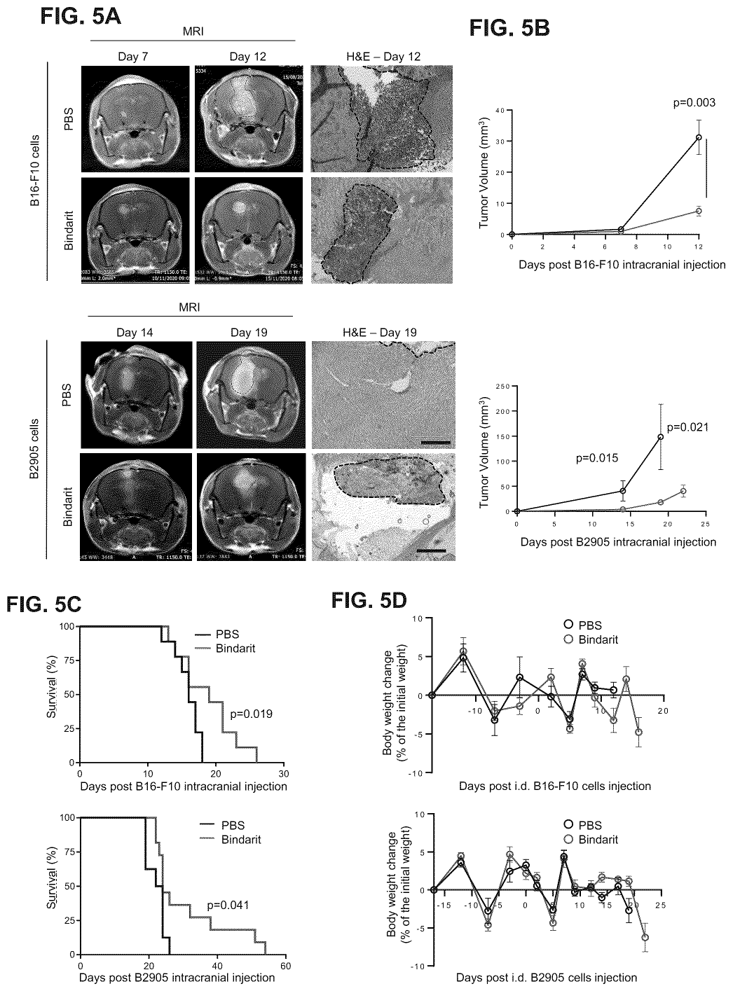

To activate the host immune system prior to direct inoculation of melanoma cells in the brain, and mimicking as faithful as possible a more realistic condition found in human patient, MBM were generated following primary melanoma (PM) establishment and resection. Although, limited by the fact that not all human patients will ultimately develop brain metastasis, the design of the in vivo models was carried out with the interventional purpose of evaluating the therapeutic efficacy of MCP-1 inhibition in MBM developed in the same location in the brain (not shown). The MBM models generated differ in their (i) tumor growth kinetics; (ii) oncogenic driver mutation; (iii) immunogenicity and therefore, response to immunotherapy. The models selected bear either BRAF-V600E oncogene mutation (D4M.3A cells) (22), PTEN-T131P onco-suppressor mutation (B16-F10 cells) (41), the RET oncogene mutation (Mel-ret cells) (42), or HGF/SF oncogenes fusion+UVB radiation (B2905 cells) (43). MBM progression was monitored and MRI scan analysis demonstrated that treatment with bindarit resulted in nearly 60-80% inhibition of tumor growth in all four MBM models examined (FIG. 4A-B and not shown), without causing any body weight loss (FIG. 5D and not shown). Bindarit-treated MBM resulted in a discrete, although significant, prolongation of the overall mice survival (FIG. 5C). The infiltration of immune cells into MBM lesions was analyzed by flow cytometry (FACS) for the presence of T-cell populations and myeloid-derived infiltrating cells (e.g., MDSC). Enhanced presence of CD8⁺ and CD4⁺ T cells was found in bindarit-treated D4M.3A, B16-F10, or Mel-ret MBM lesions (not shown). Moreover, low proliferative tumors (ki67) and high infiltration of CD8+ T cells activated (CD107) and yet not exhausted (PD-1 expression) was observed in bindarit-treated group (FIG. 4C and not shown). Bindarit inhibited the infiltration of suppressive CD4⁺ regulatory T cell (Tregs) (not shown), as well as CD11b myeloid cells with inhibitory phenotype (MDSC (CD11b⁺/Gr1⁺) and macrophages (CD11b⁺/F4/80⁺ cells) (not shown), and Iba1 microglia/macrophages recruitment in treated-MBM TME compared to those in the PBS-treated group (FIG. 4C, and not shown).

### Example 6

### Neutralization of MCP-1/CCR2 Axis by Knocking Out CCR2 Recapitulated Tumor Growth and Tumor Landscape, as Observed Following Pharmacological Inhibition of MCP-1

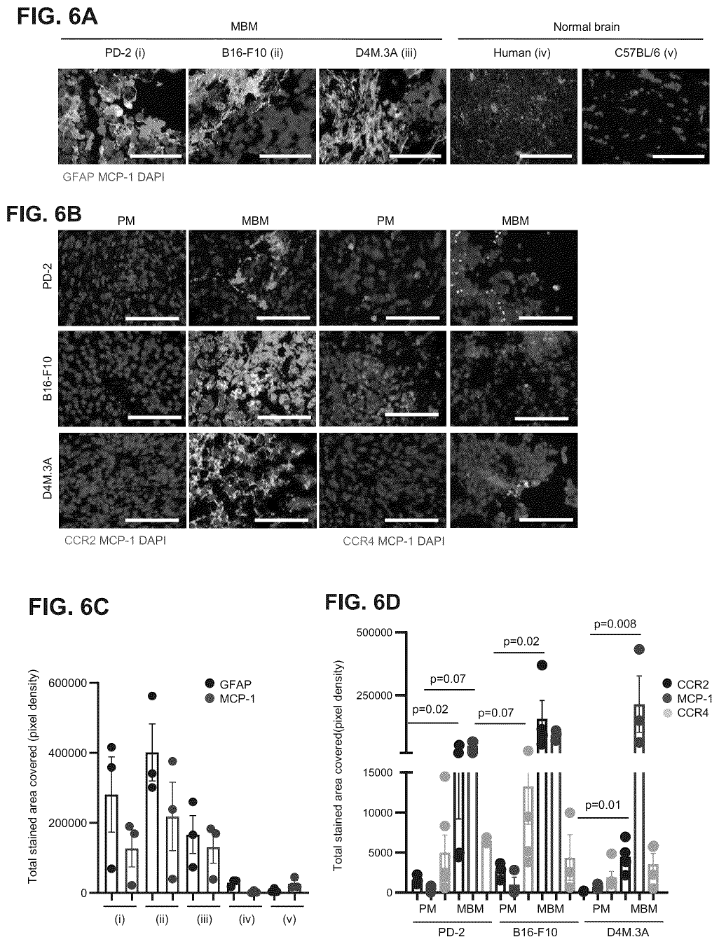

MCP-1 was highly expressed in melanoma brain tumors, including in astrocytes (confocal images and co-localization in z-stack), whereas low basal expression was found in normal healthy brain samples from human and murine tissues (FIG. 6A and not shown). Next, astrocytes-melanoma interactions via MCP-1/CCR2 axis activation, were explored by comparing the expression of CCR2 in FFPEs of human PM and MBM matched samples, or in cryosections of murine PM and in MBM models. In PM, the expression of MCP-1 and CCR2 was poor, while several regions of positive double staining were found in the metastatic lesions (FIG. 6B-C, and not shown). As MCP-1 can also bind CCR4 (30) its expression level was assessed in human and murine mouse models as well. Significantly higher expression of all three genes—CCR2, CCR4, and MCP-1—were found in MBM biopsies (from TCGA) when compared to their basal expression in healthy brain specimens of human (from GTex) and mouse (not shown). CCR4 was expressed both in PM and brain metastases, with the tendency to be highly expressed in MBM of patient-derived (PD) and D4M.3A samples. CCR2 was detected mostly in MBM samples from patients, whereas its expression was almost absent in PM samples from those patients (FIGS. 6B and 6D and not shown).

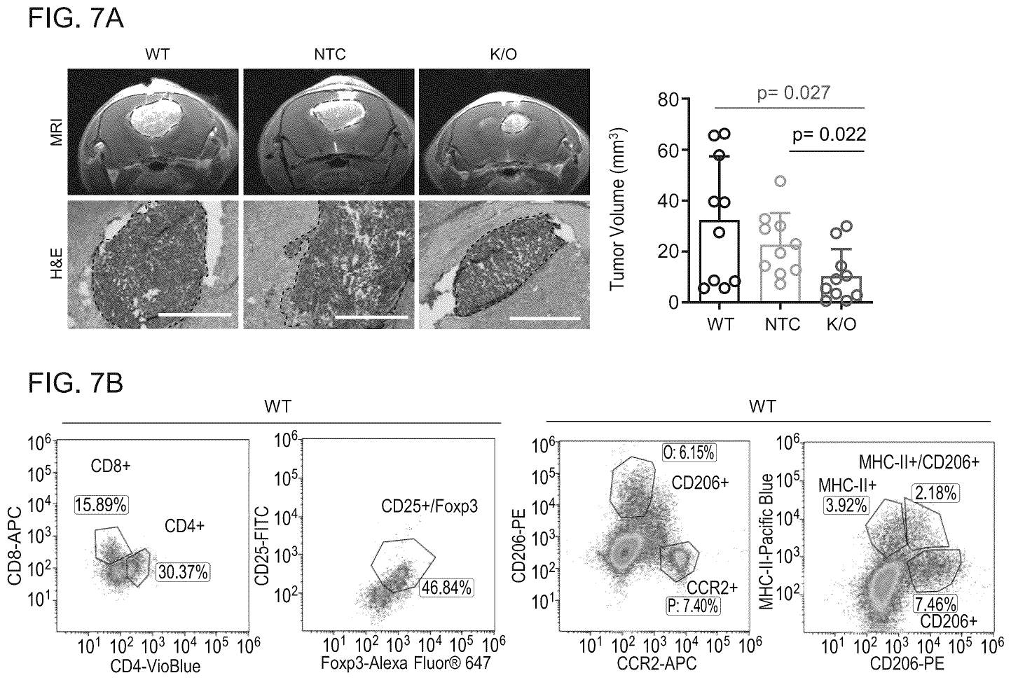

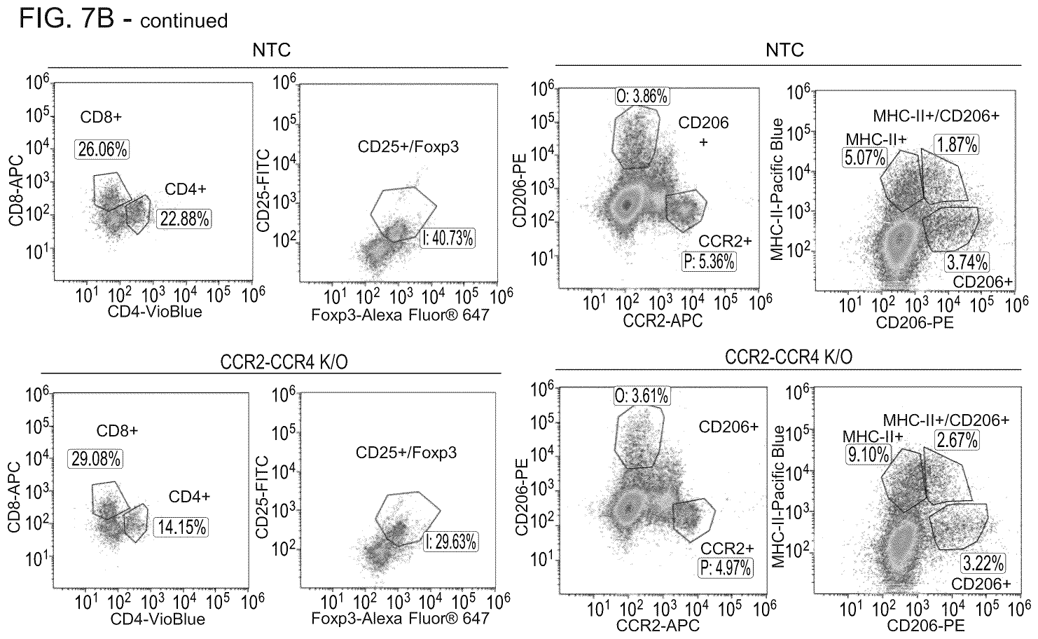

Furthermore, to understand the clinical relevance of targeting CCR2 and CCR4 receptors expressed in MBM and support the results found in matched samples of MBM and PM from human patients, the expression levels of CCR2 and CCR4 collected from CCLE/DepMap datasets of melanoma cell lines from the broad institute were plotted, where SKCM human biopsies were either derived from primary or brain metastatic sites (44). Here, CCR2 expressed by melanoma derived from the brain is 7 fold-change higher compared to melanoma in the skin, whereas 2 fold-change increased was observed for CCR4 is expression (not shown). Moreover, in vitro it was found that MCP-1 secreted from astrocytes, or recombinant murine MCP-1 (rm-MCP-1) directly induced the expression of CCR2 in B16-F10 melanoma cells (not shown), otherwise poorly expressed in the absence of MCP-1 stimulus. These findings demonstrate the induced-dependency of CCR2 expression in melanoma cells following MCP-1 stimulation, and the consequent role of this activated pathway in support of tumor cell growth, invasion, and migration. Hence, with the intention of blocking the interaction of cancer cells expressing CCR2 (and CCR4) with MCP-1 secreted in the MBM (from astrocytes and other sources of cells in the brain, including tumor cells), CCR2 and CCR4 genes were knocked out (K/O) in melanoma cells, using CRISPR/Cas9 system. B16-F10 parental cells (WT) were sorted for CCR2/CCR4 expression (not shown). CRISPR/Cas9 and guide RNA (gRNA) targeting CCR2 and CCR4 were introduced through cell electroporation. CCR2- and CCR4-negative cells were then sorted (not shown). The efficacy of CCR2 and CCR4 K/O was validated through genomic DNA sequencing, resulting in 35% and 30% gene editing, respectively (not shown). As negative control, B16-F10 cells were treated with CRISPR/Cas9 without a gRNA (NTC) and underwent sorting for CCR2⁻/CCR4⁻. Having the NTC cells also sorted for CCR2⁻/CCR4⁻ expression is an important control, as the present inventors expect this negative expression to be transient in NTC cells, in contrast to CCR2/CCR4 gRNA-treated cells where the negative expression should be stable. Finally, B16-F10 WT, NTC, and CCR2/CCR4 K/O MBM were generated following intracranial cell inoculation. On day 9, MRI scan analyses demonstrated that K/O melanoma tumors developed into less aggressive brain lesions (FIG. 7A). Moreover, the present inventors observed a moderate increase in overall mice survival (not shown). Histological analysis of double K/O tumors presented decreased tumor associated-CCR2 and CCR4 expression, and poor macrophages (F4/80 cells)-associated CCR2 expression. Moderate reduction in GFAP activation was observed along with reduced tumor proliferation (Ki-67), and lower levels of microglia/macrophages Iba1⁺ cells (not shown). FACS analysis demonstrate higher infiltration of CD8⁺ T cells and reduced presence of CD4⁺CD25⁺Foxp3⁺ (Treg) into double K/O tumors (30-40% reduction compared to NTC and WT) (FIG. 7B). Furthermore, macrophages/microglia (CD11b⁺/F4/80⁺) in K/O tumors showed higher levels of the antigen presenting marker MHC-II, and lower expression of the immunosuppressive markers CD206 and CCR2, compared to WT and NTC control tumors (FIG. 7B).

### Example 7

### MCP-1/CCR2 Axis Promotes Brain Metastases in Breast Cancer Brain Metastasis

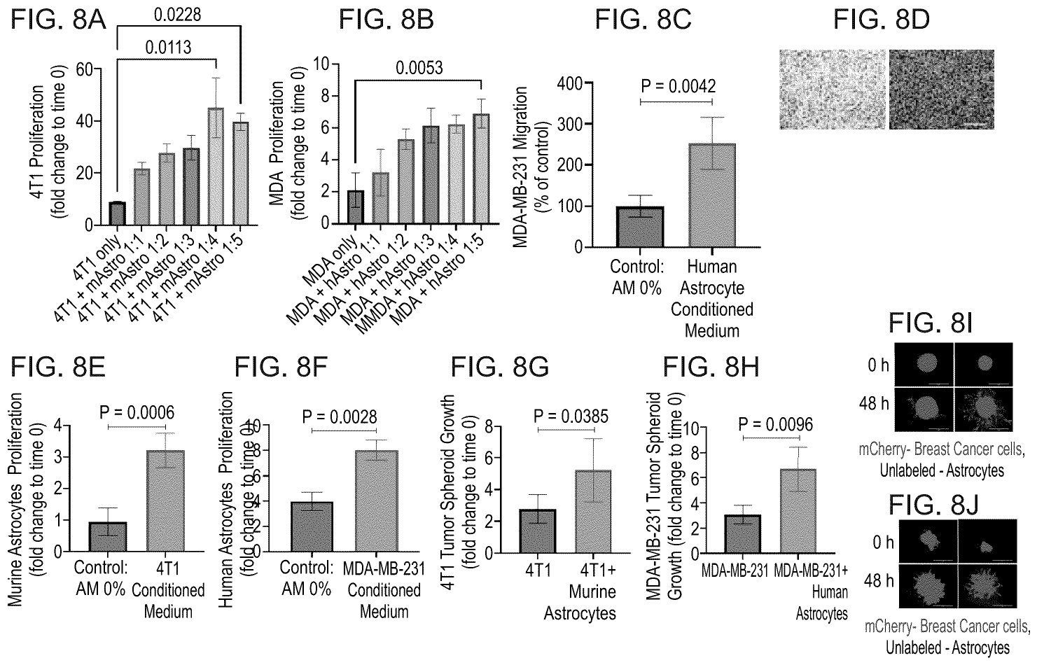

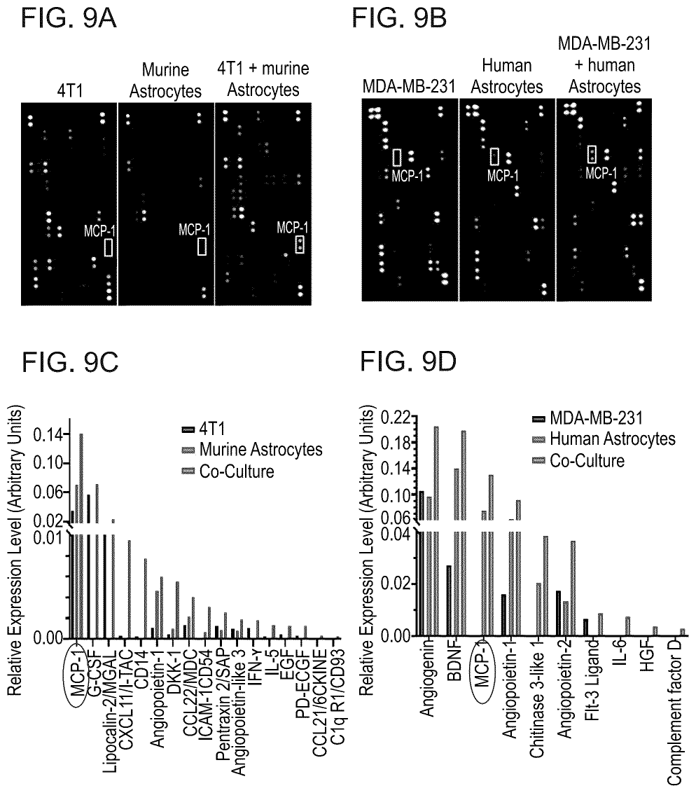

To further uncover the role of MCP-1 in brain metastases, its involvement in breast cancer-brain microenvironment was studied. First, to evaluate the effect of the brain stromal cells on breast cancer cell properties and vice versa, transwell migration, proliferation and 3D multicellular tumor spheroid assays were conducted. An increase in the proliferation rate of breast cancer cell in the presence of astrocytes (FIGS. 8A-B), and an increase of the astrocytes proliferation rate when exposed to breast cancer cell CM (FIGS. 8E-F) were observed. In addition, breast cancer cell migration was enhanced towards astrocyte CM (FIGS. 9C-D). Furthermore, when incorporated with astrocytes, breast cancer cell 3D multicellular spheroids have shown an increased invasion (FIGS. 8G-J).

After finding the reciprocal effects of astrocytes and breast cancer, the present inventors aimed to identify secreted factors involved in this intercommunication. Cytokine arrays were performed and a set of up-regulated cytokines in the co-culture settings was found (FIGS. 9A-D), among them MCP-1 was one of the most highly secreted factors and was upregulated both in the human and the murine cell lines.

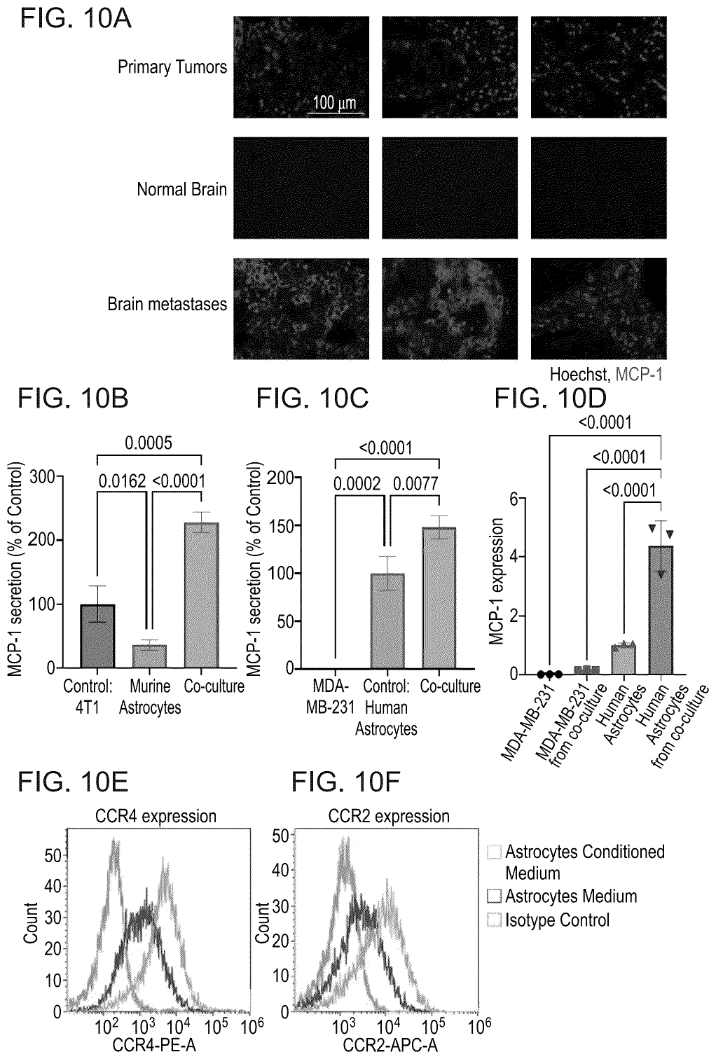

In order to further validate the expression of MCP-1, ELISA assays were performed for the protein secretion level. In agreement with the results from the cytokine arrays, higher secretion of MCP-1 was demonstrated when breast cancer cells were co-cultured with astrocytes, compared to the mono-cultures. In addition, 4T1 cells secreted higher levels of MCP-1 compared to the level secreted by murine astrocytes, while human astrocytes secreted higher levels of the protein compared to MDA-MB-231 cells, in agreement with the cytokine arrays (FIG. 10B-C).

The mRNA expression level of MCP-1 was further validated using RT-qPCR. MDA-MB-231 cells and human astrocytes were mono- or co-cultured, and then sorted by fluorescence. It was found that even though the astrocytes expressed much higher levels of MCP-1, both the cell types expressed higher levels of MCP-1 when they were co-cultured, compared to growing each of them separately (FIG. 10D).

In addition, the present inventors wanted to assess the expression levels of the receptors for CCL2 by flow cytometry. CCR2 and CCR4 were found to be expressed on the surface of 4T1 cells that were grown either in AM or in astrocyte CM (FIG. 10E-F).

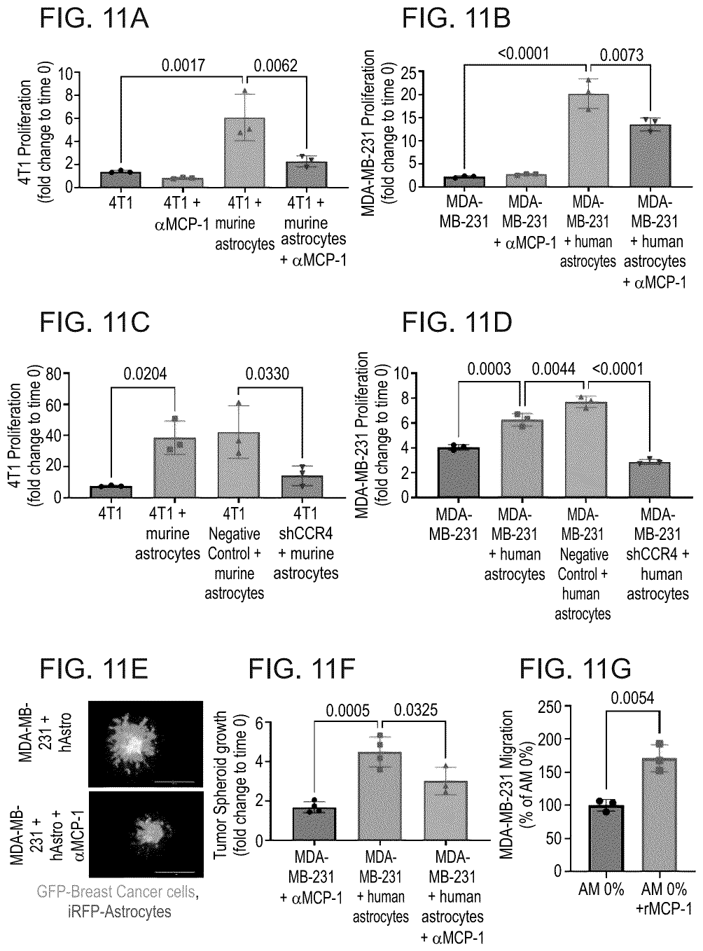

The relevance of MCP-1 was validated by immunohistochemistry staining on FFPE clinical samples of brain metastases from breast cancer. MCP-1 is highly expressed in the brain metastases compared to the primary tumors and normal brain (FIG. 11A), suggesting it is also relevant to human patients.

In order to test whether MCP-1 is important and to evaluate its role in the interactions between breast cancer cells and astrocytes, proliferation, migration and spheroids experiments were performed in the presence of MCP-1 recombinant protein and/or neutralizing antibody. First, proliferation assays of breast cancer cells co-cultured with astrocytes or mono-cultured were performed. In both cases, when the cells were treated with MCP-1 neutralizing antibody, breast cancer cell proliferation decreased (FIG. 11A-B). Similarly, the proliferation rate decreased when the CCR4 receptor was knocked down using shRNA system (FIG. 11C-D). Moreover, in a transwell migration assay, MDA-MB-231 breast cancer cells migrated more towards MCP-1 recombinant protein compared to the control (FIG. 11G). Tumor spheroids experiments showed a decrease in spheroid growth and sprouting in the presence of MCP-1 neutralizing antibody (FIG. 11E-F).

### Example 8

### MCP-1/CCR2-CCR4, P-Selectin/P-Selectin Ligand-1, PD-PD-L1 Axes are Upregulated in Human BCBM

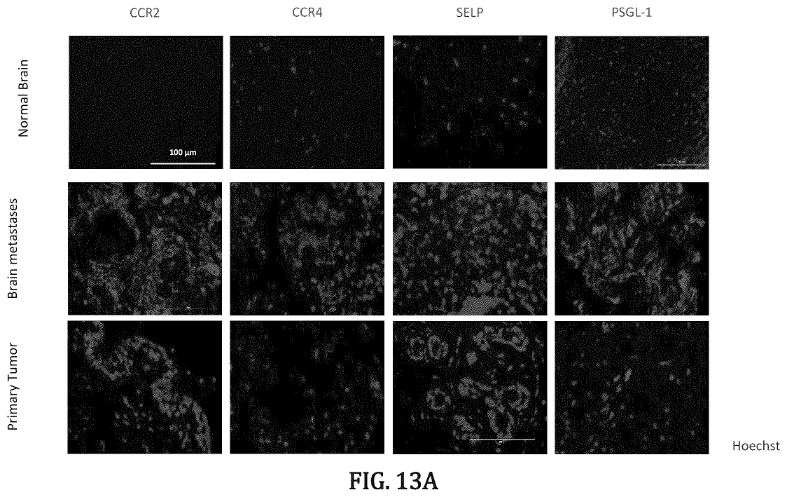

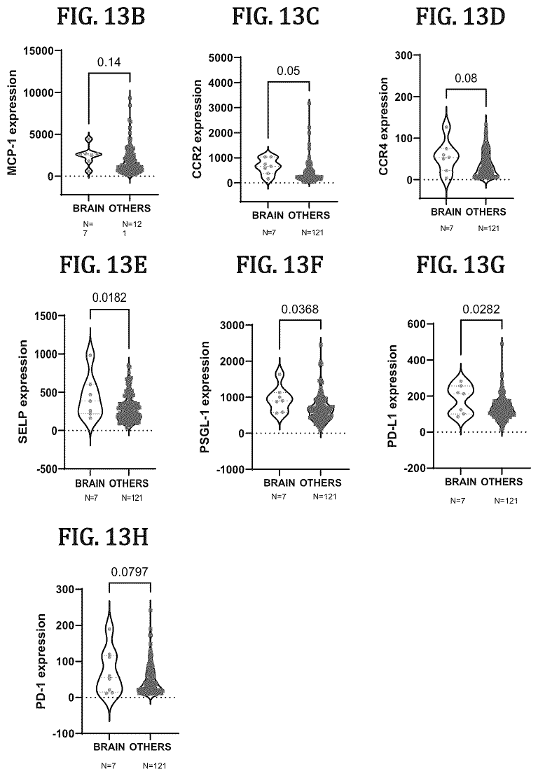

The present inventors validated the relevance of the MCP-1/CCR2-CCR4 axis by immunohistochemistry (IHC) staining on formalin-fixed paraffin-embedded (FFPE) clinical samples of breast cancer brain metastases (BCMB). The present inventors found that CCR2 is highly overexpressed in brain metastasis (BM) compared to primary breast cancer (BC) and normal brains (FIG. 13A). Even though CCR4 presented generally lower expression levels than CCR2, it was also overexpressed in clinical BM samples compared to primary BC and to normal brain tissues (FIG. 13A). The present inventors further analyzed the their expression levels using data from the GSE12276 dataset, in which samples were collected from primary tumors of BC patients that later metastasized either to the brain or to other organs. Even though not all of them were statistically significant, MCP-1, CCR2, and CCR4, showed a trend of higher expression in BC that metastasized later on to the brain (BCMB) (FIG. 13B-D).

As BC is highly aggressive and frequently metastasize to the brain, the present inventors decided to include additional targets to be combined with MCP-1 inhibition in BCBM, that are involved in the interactions with other tumor microenvironment components.

Our previous study, focused on the crosstalk between glioblastoma and microglia cells, showed that the P-selectin (SELP) expressed by glioblastoma cells and P-selectin ligand-1 (PSGL-1) expressed by microglia cells alters microglia/macrophages activation phenotype and led to enhanced glioblastoma proliferation and invasion. Thus, the inhibition of SELP reduced tumor growth and increased survival in glioblastoma mouse model (45).

Indeed, the present inventors performed IHC staining of patient tissue sections to assess the expression of SELP and PSGL-1 in BCMB. The present inventors found that they are highly upregulated in BCBM compared to their expression in BC and normal brains (FIG. 13A). Further validation of RNA expression from GSE12276 dataset collected from patients, also showed an increased expression of SELP and PSGL-1 in primary tumors that later metastasized to the brain compared to tumors that metastasized to other organs (FIG. 13E-F).

Moreover, immune checkpoint inhibitor (ICIs) targeting programmed cell death protein-1 (PD-1) or its ligand (PD-L1) have demonstrated intracranial effects in BM originating from melanoma (MBM) and non-small cell lung cancer (NSCLC)(46, 47). ICIs also show promising outcome in the treatment of metastatic breast cancer(48). However, there is a lack of data regarding the efficacy in BC patients with BM, as these are often excluded from clinical trials due to concerns about toxicities, limited efficacy of systemic agents across the BBB and overall poor prognosis(48, 49). Nevertheless, the present validation from data from GSE12276 dataset collected from patients, revealed that PD-1 (significantly) and PD-L1 (non-significantly) have higher levels in primary BCtumors that later metastasized to the brain compared to tumors that metastasized to other organs (FIG. 13G-H).

### Example 9

### MCP-1-CCR2/4 Axis is Important for Breast Cancer Proliferation, Migration and Invasion

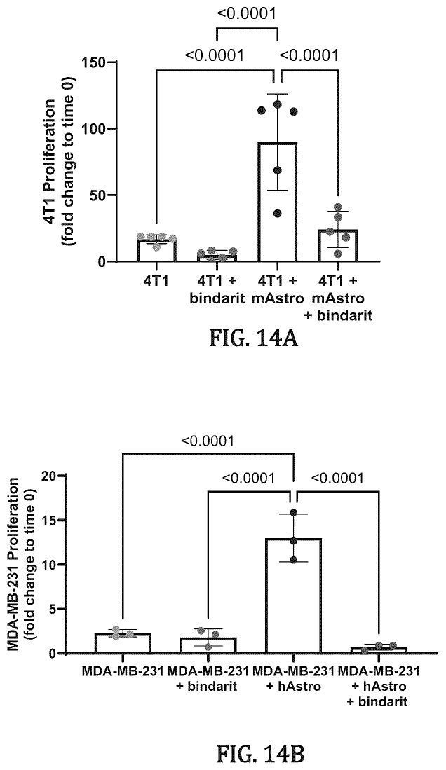

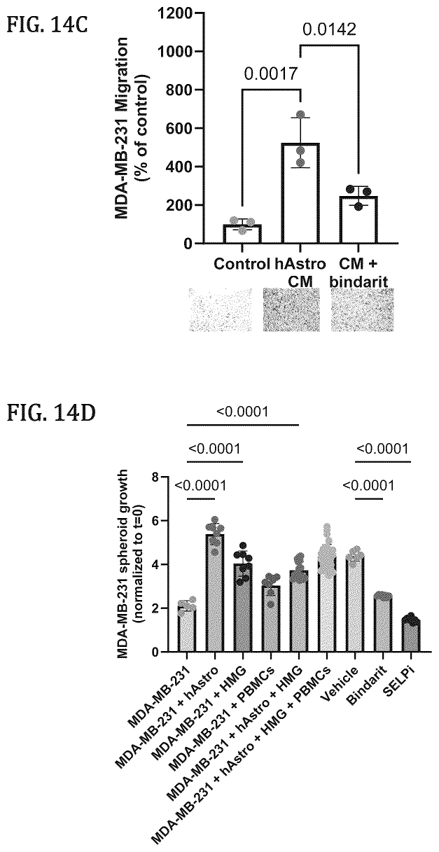

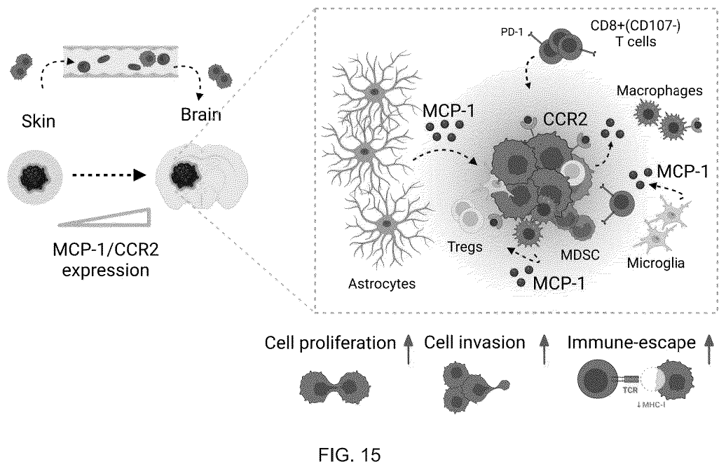

As previously showed in MBM (50), in order to test whether MCP-1 is important and to evaluate its role in the crosstalk between BC cells and astrocytes, the present inventors performed proliferation, migration and spheroids experiments in the presence of an MCP-1 small molecular weight inhibitor bindarit. First, the present inventors performed proliferation assays of MDA-MB-231 and 4T1 cells, co-cultured with astrocytes or mono-cultured. In both cases, when the cells were treated with bindarit (MCP-1 inhibitor), BC cell proliferation dramatically decreased, although a superior effect on the inhibition of cell proliferation was achieved following BC and astrocytes co-culture (FIG. 14A-B). In addition, using a transwell migration assay, bindarit significantly decreased the migration rate of MDA-MB-231 cells towards human astrocyte CM (FIG. 14C). The present inventors further performed 3D tumor spheroids experiments, containing either BC cells alone, or BC cells and astrocytes, microglia, and peripheral blood mononuclear cells (PBMCs). Utilizing this model, the present inventors observed a significant decrease in spheroid size in the presence of bindarit or a SELP small molecular molecule inhibitor (SELPi) (FIG. 14D).

### Example 10

### MCP-1/CCR2 Axis is Upregulated in Lung Cancer Brain Metastasis

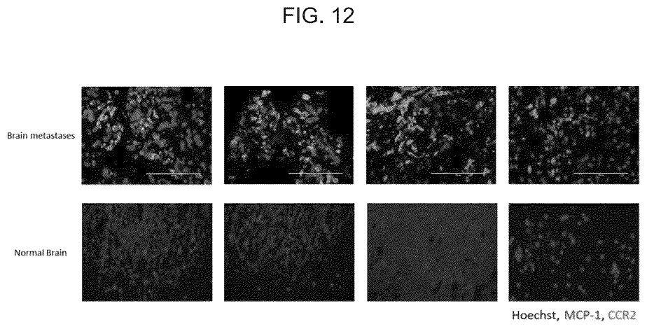

The relevance of MCP-1 was validated by immunohistochemistry staining on FFPE clinical samples of brain metastases from lung cancer. It was found that MCP-1 is highly expressed in the brain metastases compared to normal brain (FIG. 12), suggesting it is pivotal to the metastatic process of lung cancer to the brain.

### Example 11

### Combination Therapy Bindarit and Either P-Selectin Inhibitor or αPD-1 Antibody, Leads to Favorable Outcomes in Orthotopic Breast Cancer Brain Metastases Model

The present inventors tested bindarit, SELPi, and αPD-1 antibody, each alone or in different combinations, using immune competent BALB\c mouse model. To model the disease course in vivo, as closely as possible to the human disease in which metastases develop only after a primary tumor was established, 4T1 cells were first inoculated orthotopically to the mammary fat pad, and the tumors were later on resected. The existence of a primary tumor, is also necessary in order to achieve an intracranial response to ICIs, as previously shown in a melanoma brain metastases model (51). Following the resection of the primary tumor, 4T1 cells were injected intracranially into the brain, and mice were treated with the drugs for two weeks.

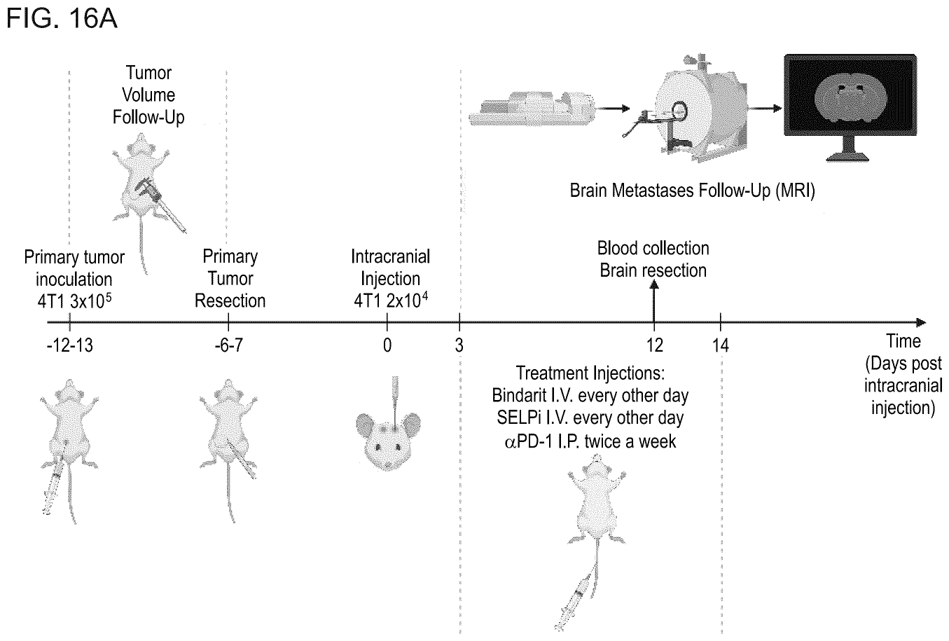

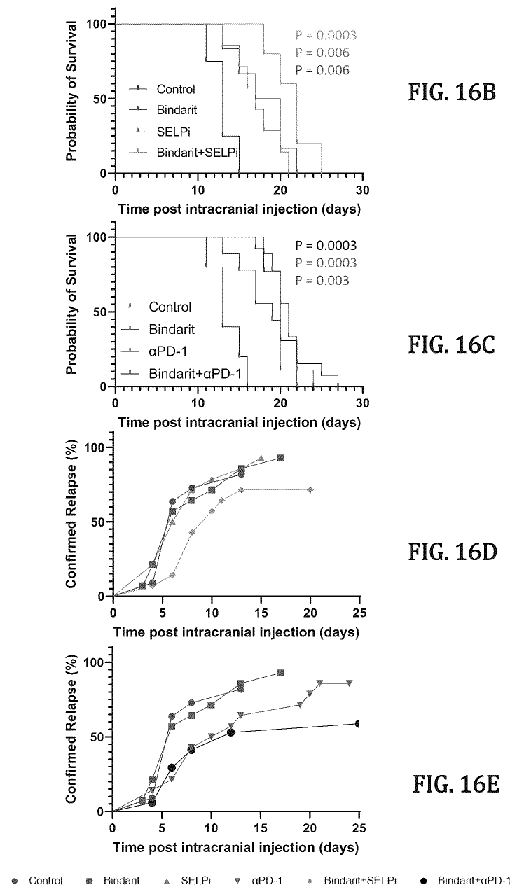

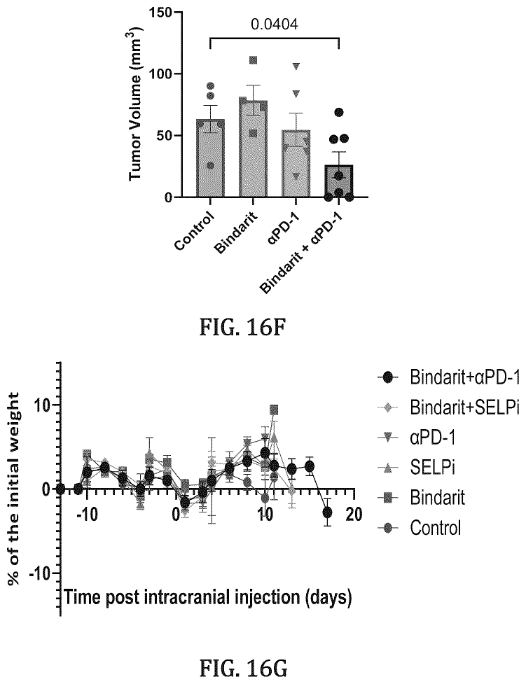

Each of the drugs significantly increased mice survival, compared to the control group. Combining bindarit with either of the other targets has led to a further increase in overall survival (FIG. 16B-C). Analysis of the systemic disease relapse, presented by primary tumor recurrence or detection of extracranial metastases, revealed another advantage of the combination therapies. While bindarit and SELPi each alone did not show any improvement in relapse in comparison to the control group, the combination of both drugs presented a delay in the detection of any relapse, and a decrease in overall percent of mice presenting extracranial metastases or tumor recurrence (FIG. 16D). In the case of the ICI, αPD-1 antibody did not reduced tumor relapse compared to the control group. However, when combining αPD-1 antibody with bindarit, the efficacy of ICI was further improved (FIG. 16E). The combination of bindarit with αPD-1 has also led to a reduction in BM volume (FIG. 16F).

### Example 12

### Combination Therapy of Bindarit and Nanoparticles Containing BRAF\MEK Inhibitors, Leads to Reduction in Melanoma Brain Metastases 3D Spheroid Invasion

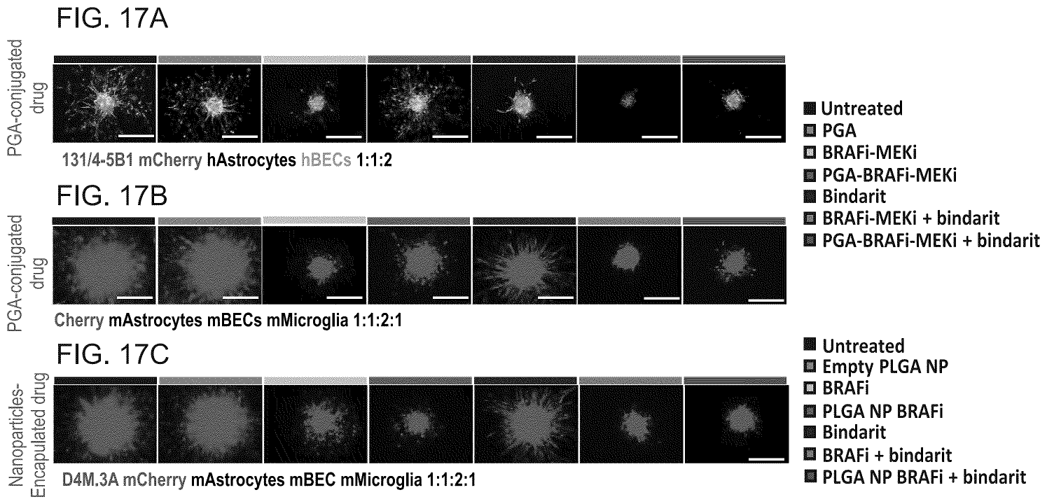

As previously shown, the anti-tumor efficacy of BRAF and MEK inhibitors (BRAFi and MEKi) against melanoma proliferation and invasion was improved by either conjugating BRAFi and MEKi to a biodegradable poly(α,1-glutamic acid) (PGA) (52) or encapsulating BRAFi into poly lactic-co-glycolic acid, (PLGA) nanoparticles (NPs). The present inventors thus assessed the efficacy of a combination therapy of bindarit and the thereof BRAFi and MEKi in 3D MCTS consisting of melanoma cells and brain resident stromal cells (astrocytes, brain endothelial cells and microglia). The combined treatment of bindarit together with either PGA-conjugated drugs or PLGA-nanoparticles, resulted in the highest inhibition of melanoma cell invasion (mCherry labeled-cells) compared to the monotherapy or to the combination of bindarit and free-drugs (FIG. 17A-C).

### Example 13

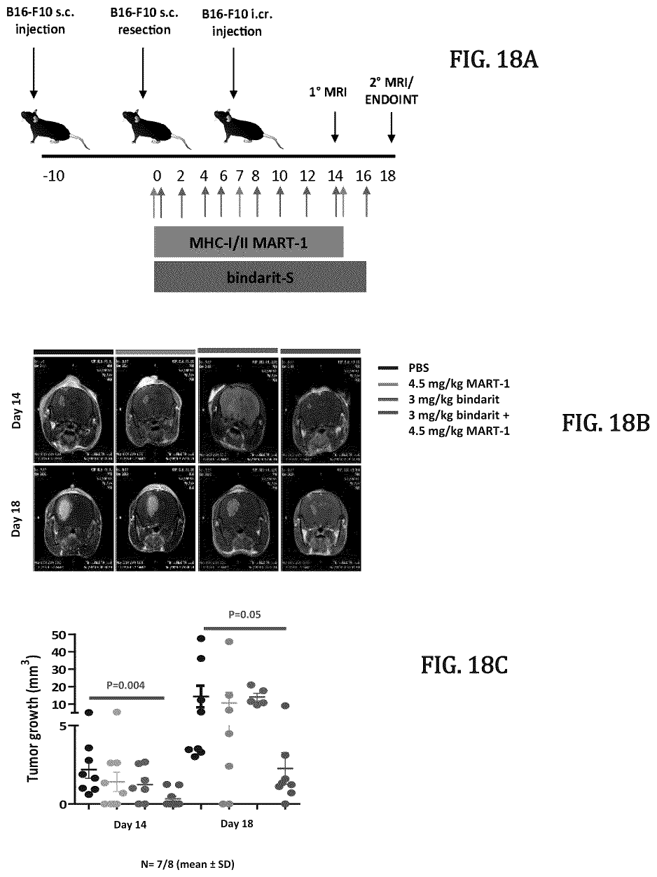

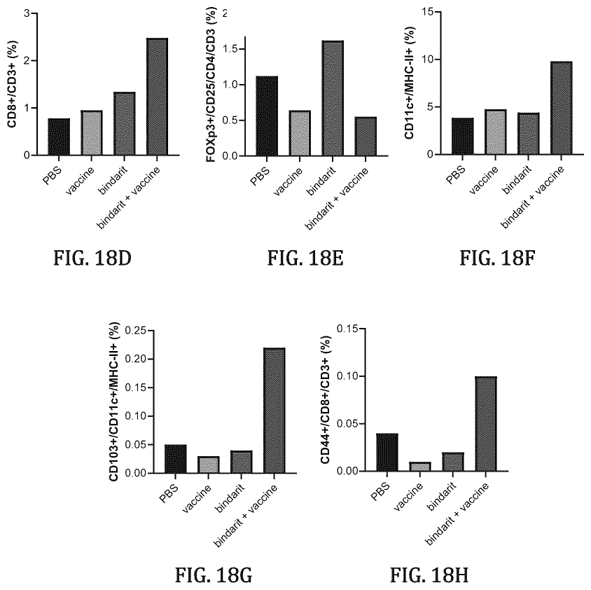

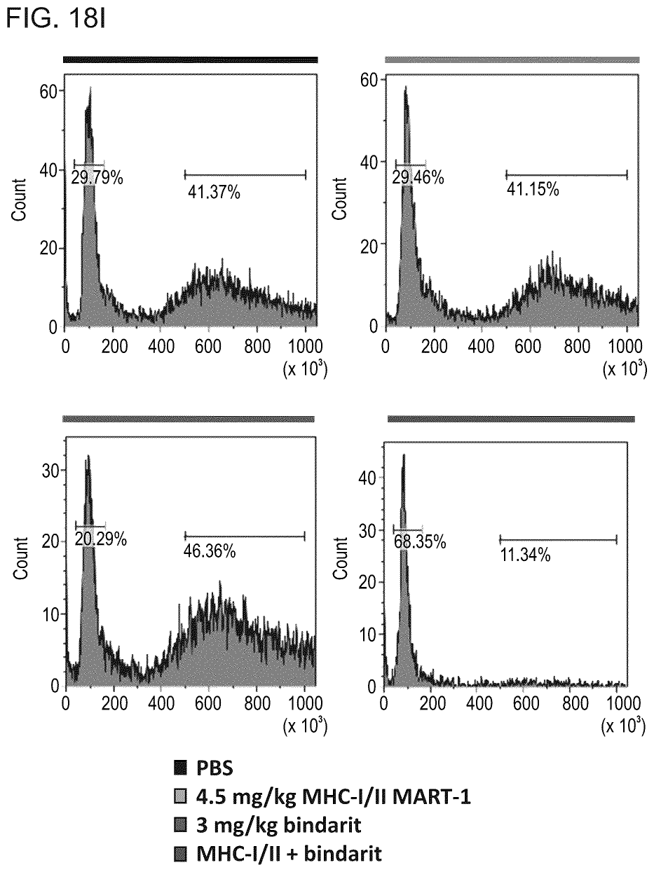

Combination therapy of bindarit and nanovaccine, leads to favorable outcomes in orthotopic melanoma brain metastases model As the present inventors previously demonstrated that bindarit administration increased the infiltration of cytotoxic CD8⁺ T cells and attenuates the immunosuppressive phenotype of the brain microenvironment (BME) in several models of intracranially-injected MBM (50), while dendritic cell-targeted nanovaccines stimulated T-cell infiltration into primary melanoma tumors (19), the present inventors assessed the synergism of combining bindarit and nanovaccine in preventing MBM. B16-F10 melanoma cells where inoculated subcutaneously (100.000 cells), and the tumor where resected when reached an average size 100-150 mm³, prior the generation of MBM as previously shown (50). Ten days post resection of the primary tumor, mice were randomized and injected i.v. with one dose of bindarit (3 mg/kg) every other day (QOD) for 16 days and intranasal administration of nanovaccine containing MART-1 peptides (4.5 mg/kg) or PBS ones a week for three weeks. After primary tumor resection, B16-F10 melanoma cells (15.000 cells) were injected intracranially and mice where continuous treated (FIG. 18A). MRI quantification of tumor growth at day 14 and 18 post B16-F10 intracranially-injected cells, revealed that the group treated with the combination of nanovaccine and bindarit presented a significant reduction in tumor size compared to PBS or mono-treatment groups, even though bindarit was administered at the dose of 3 mg/kg instead of 100 mg/kg as previously shown (50). Indeed the tumor size of bindarit at 3 mg/kg or nanovaccine alone was comparable to that of PBS-treated group, showing a remarkable synergistic effect of the combination (FIG. 18B-C). Thus, changes in the infiltration of immune cells into B16-F10 MBM were assessed followed tumor resection at day 18. The combination of nano-vaccine and bindarit showed higher infiltration of cytotoxic T cells, antigen presenting cells (APC), tissue-resident memory T cells (TRM), effector T cells, and lower infiltration of regulatory T cells (Tregs) compared to PBS or the mono-treatment groups (FIG. 18D-H). Moreover, MART-1⁺ melanoma cell cycle resulted to be arrested at phase GO/1 following combination of nanovaccine and bindarit (FIG. 18I).

### Example 14

### Combination Therapy of Bindarit and Immune Checkpoint Inhibitors, Following Nanovaccine Immunization, Leads to Reduced Melanoma 3D Spheroids Invasion

In order to predict ex vivo possible synergistic combinations of nanovaccine and bindarit together with ICI, animals were treated as previously described (FIG. 18A). B16-F10 melanoma cells where inoculated subcutaneously (100.000 cells), and the tumor where resected when reached an average size 100-150 mm³, prior the generation of MBM as previously shown (50). Ten days post resection of the primary tumor, mice were randomized and injected i.v. with one dose of bindarit (3 mg/kg free drug) every other day (QOD) for 16 days and intranasal administration of nano-vaccine containing mart-1 peptides (4.5 mg/kg) or PBS ones a week for three weeks. After primary tumor resection, B16-F10 melanoma cells (15.000 cells) were injected intracranially and mice where continuous treated (FIG. 18A). Splenocytes from animal treated with bindarit and PBS were then isolated and activated with plain activation (α-CD28 and β-mercapto), whereas splenocytes from animal treated with bindarit and nanovaccine were additionally exposed to MHCI/II MART-1 peptides for a week (FIG. 19A). Then, 3D MCTS consisting of melanoma B16-F10 cells and murine astrocytes were co-cultured with the activated splenocytes and exposed to a treatment combination of ICI (α-PD-1, and α-OX40) and bindarit (FIG. 19A). In the presence of splenocytes-derived from vaccinated animals with bindarit and nanovaccine, the treatment of α-PD-1 alone, or in combination with α-OX40 and bindarit significantly reduced the invasion ability of melanoma cells compared that to splenocytes-derived from vaccinated animals with bindarit and PBS (FIG. 19B-C).

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the Applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

### REFERENCES

1. S. Goyal et al., Clinical Management of Multiple Melanoma Brain Metastases: A Systematic Review. JAMA Oncol 1, 668-676 (2015).
2. C. Durio, M. Veiga, N. Pedro, G. Manuela, F. Pedrosa, Sudden death associated with melanoma brain metastases. Journal of Pathology of Nepal 8, 1412 (2018).
3. F. Philip, Advances in the Systemic Treatment of Melanoma Brain Metastases. 10.5772/intechopen.85673 (2020).
4. E. Vosoughi et al., Survival and clinical outcomes of patients with melanoma brain metastasis in the era of checkpoint inhibitors and targeted therapies. BMC Cancer 18, 490-490 (2018).
5. A. M. Di Giacomo et al., Immunotherapy of brain metastases: breaking a “dogma”. Journal of Experimental & Clinical Cancer Research 38, 419 (2019).
6. M. Harary, D. A. Reardon, J. B. Iorgulescu, Efficacy and safety of immune checkpoint blockade for brain metastases. CNS Oncology 8, CNS33 (2019).
7. H. A. Tawbi, C. Boutros, D. Kok, C. Robert, G. McArthur, New Era in the Management of Melanoma Brain Metastases. American Society of Clinical Oncology Educational Book 10.1200/edbk_200819, 741-750 (2018).
8. D. F. Quail, J. A. Joyce, Microenvironmental regulation of tumor progression and metastasis. Nature Medicine 19, 1423-1437 (2013).
9. D. Wasilewski, N. Priego, C. Fustero-Torre, M. Valiente, Reactive Astrocytes in Brain Metastasis. Front Oncol 7 (2017).
10. H. Doron et al., Inflammatory Activation of Astrocytes Facilitates Melanoma Brain Tropism via the CXCL10-CXCR3 Signaling Axis. Cell Reports 28, 1785-1798.e1786 (2019).
11. A. Boire, P. K. Brastianos, L. Garzia, M. Valiente, Brain metastasis. Nature Reviews Cancer 20, 4-11 (2020).
12. T. Redmer, Deciphering mechanisms of brain metastasis in melanoma—the gist of the matter. Mol Cancer 17, 106-106 (2018).
13. K. I. Ataga et al., Crizanlizumab for the Prevention of Pain Crises in Sickle Cell Disease. N Engl J Med 376, 429-439 (2017).
14. P. W. Bedard et al., Characterization of the novel P-selectin inhibitor PSI-697 [2-(4-chlorobenzyl)-3-hydroxy-7,8,9,10-tetrahydrobenzo[h] quinoline-4-carboxylic acid] in vitro and in rodent models of vascular inflammation and thrombosis. J Pharmacol Exp Ther 324, 497-506 (2008).
15. I. Y. Park, D. S. Lee, M. H. Song, W. Kim, J. M. Won, Cylexin: a P-selectin inhibitor prolongs heart allograft survival in hypersensitized rat recipients. Transplant Proc 30, 2927-2928 (1998).
16. F. B. Mayr et al., Effects of the pan-selectin antagonist bimosiamose (TBC1269) in experimental human endotoxemia. Shock 29, 475-482 (2008).
17. J. Chang et al., GMI-1070, a novel pan-selectin antagonist, reverses acute vascular occlusions in sickle cell mice. Blood 116, 1779-1786 (2010).
18. L. Brandolini et al., DF2726A, a new IL-8 signalling inhibitor, is able to counteract chemotherapy-induced neuropathic pain. Scientific Reports 9, 11729 (2019).
19. J. Conniot et al., Immunization with mannosylated nanovaccines and inhibition of the immune-suppressing microenvironment sensitizes melanoma to immune checkpoint modulators. Nature Nanotechnology 14, 891-901 (2019).
20. E. Segal et al., Targeting angiogenesis-dependent calcified neoplasms using combined polymer therapeutics. PloS one 4, e5233-e5233 (2009).
21. W. Cruz-Munoz, S. Man, P. Xu, R. S. Kerbel, Development of a Preclinical Model of Spontaneous Human Melanoma Central Nervous System Metastasis. Cancer Research 68, 4500-4505 (2008).
22. M. H. Jenkins et al., Multiple murine BRaf(V600E) melanoma cell lines with sensitivity to PLX4032. Pigment Cell Melanoma Res 27, 495-501 (2014).
23. S. van Neerven et al., Inflammatory cytokine release of astrocytes <em>in vitro</em> is reduced by all—<em>trans</em> retinoic acid. Journal of neuroimmunology 229, 169-179 (2010).
24. S. Ferber et al., Co-targeting the tumor endothelium and P-selectin-expressing glioblastoma cells leads to a remarkable therapeutic outcome. eLife 6, e25281 (2017).
25. D. Y. Guschin et al., A rapid and general assay for monitoring endogenous gene modification. Methods Mol Biol 649, 247-256 (2010).
26. B. K. Lipska et al., Critical factors in gene expression in postmortem human brain: Focus on studies in schizophrenia. Biological psychiatry 60, 650-658 (2006).
27. A. Klein et al., Astrocytes facilitate melanoma brain metastasis via secretion of IL-23. The Journal of Pathology 236, 116-127 (2015).
28. Q. Lin et al., Reactive astrocytes protect melanoma cells from chemotherapy by sequestering intracellular calcium through gap junction communication channels. Neoplasia 12, 748-754 (2010).
29. H. Schwartz et al., Incipient Melanoma Brain Metastases Instigate Astrogliosis and Neuroinflammation. Cancer Research 76, 4359-4371 (2016).
30. A. Klein et al., CCR4 is a determinant of melanoma brain metastasis. Oncotarget 8, 31079-31091 (2017).
31. D. Li et al., Neurochemical regulation of the expression and function of glial fibrillary acidic protein in astrocytes. Glia 68, 878-897 (2020).
32. A. Morsi et al., Development and characterization of a clinically relevant mouse model of melanoma brain metastasis. Pigment Cell Melanoma Res 26, 743-745 (2013).
33. M. Nakai et al., Establishment of a murine model for metastasis of cytokine-producing tumor to the brain. Pigment Cell Res 10, 304-309 (1997).
034. H. Aucott et al., Neuroinflammation in Response to Intracerebral Injections of Different HMGB1 Redox Isoforms. Journal of innate immunity 10, 215-227 (2018).
35. M. Valiente et al., Brain Metastasis Cell Lines Panel: A Public Resource of Organotropic Cell Lines. Cancer Research 80, 4314-4323 (2020).
36. B. Nash, K. Ioannidou, S. C. Barnett, Astrocyte phenotypes and their relationship to myelination. Journal of anatomy 219, 44-52 (2011).
37. S. van Neerven et al., Inflammatory cytokine release of astrocytes in vitro is reduced by all-trans retinoic acid. J Neuroimmunol 229, 169-179 (2010).
38. E. Mora, A. Guglielmotti, G. Biondi, P. Sassone-Corsi, Bindarit: an anti-inflammatory small molecule that modulates the NFκB pathway. Cell cycle (Georgetown, Tex.) 11, 159-169 (2012).
39. A. S. Berghoff et al., Tumour-infiltrating lymphocytes and expression of programmed death ligand 1 (PD-L1) in melanoma brain metastases. Histopathology 66, 289-299 (2015).
40. R. Satchi-Fainaro et al., Inhibition of vessel permeability by TNP-470 and its polymer conjugate, caplostatin. Cancer Cell 7, 251-261 (2005).
41. W. Zhong et al., Comparison of the molecular and cellular phenotypes of common mouse syngeneic models with human tumors. BMC Genomics 21, 2 (2020).
42. M. Taniguchi et al., Establishment and characterization of a malignant melanocytic tumor cell line expressing the ret oncogene. Oncogene 7, 1491-1496 (1992).
43. E. Pérez-Guijarro et al., Multi-model preclinical platform predicts clinical response of melanoma to immunotherapy. bioRxiv 10.1101/727826, 727826 (2019).
44. A. Tsherniak et al., Defining a Cancer Dependency Map. Cell 170, 564-576.e516 (2017).
45. E. Yeini et al., P-selectin axis plays a key role in microglia immunophenotype and glioblastoma progression. Nature communications 12, 1-22 (2021).
46. S. B. Goldberg et al., Pembrolizumab for management of patients with NSCLC and brain metastases: long-term results and biomarker analysis from a non-randomised, open-label, phase 2 trial. The Lancet Oncology 21, 655-663 (2020).
47. H. A. Tawbi et al., Combined nivolumab and ipilimumab in melanoma metastatic to the brain. New England Journal of Medicine 379, 722-730 (2018).
48. D. B. Page et al., Brain radiotherapy, tremelimumab-mediated CTLA-4-directed blockade+/− trastuzumab in patients with breast cancer brain metastases. NPJ Breast Cancer 8, 50 (2022).
49. I. Schlam, M. E. Gatti-Mays, Immune Checkpoint Inhibitors in the Treatment of Breast Cancer Brain Metastases. The Oncologist 27, 538-547 (2022).
50. S. Pozzi et al., MCP-1/CCR2 axis inhibition sensitizes the brain microenvironment against melanoma brain metastasis progression. JCI Insight 7 (2022).
51. D. Taggart et al., Anti-PD-1/anti-CTLA-4 efficacy in melanoma brain metastases depends on extracranial disease and augmentation of CD8⁺ T cell trafficking. Proceedings of the National Academy of Sciences 115, E1540-E1549 (2018).
52. E. Pisarevsky et al., Rational Design of Polyglutamic Acid Delivering an Optimized Combination of Drugs Targeting Mutated BRAF and MEK in Melanoma. Advanced Therapeutics, 2000028.

What is claimed is:

1. A method of treating brain metastasis in a subject in need thereof, the method comprising administering to the subject a therapeutically effective amount of an inhibitor of an MCP-1/CCR2/CCR4 axis, thereby treating the brain metastasis in the subject.

2. A method of activating an immune response against brain metastasis in a subject in need thereof, the method comprising administering to the subject a therapeutically effective amount of an inhibitor of an MCP-1/CCR2/CCR4 axis, thereby activating the immune response against brain metastasis in the subject.

3. The method of claim 2, wherein said activating said immune response is by at least one of:
(i) inducing polarization or activation of immunosuppressive immune cells (e.g., macrophages) from a pro-tumorogenic anti-inflammatory phenotype towards an immune-potentiator pro-inflammatory anti-tumorogenic phenotype; and
(ii) inducing infiltration of CD8⁺ T cells to said brain metastasis.

4. The method of claim 3, wherein said immunosuppressive immune cells comprise macrophages.

5. The method of claim 1, wherein said brain metastasis is metastatic brain melanoma (MBM), metastatic breast cancer, metastatic gastrointestinal (GI) cancer, metastatic lung cancer, metastatic brain melanoma (MBM) metastatic brain lung cancer or metastatic brain breast cancer.

6. The method of claim 1, wherein said subject is post-surgery of the primary tumor for prevention and for treatment of surgically-removed brain metastases if resectable or for regression, if not resectable.

7. The method of claim 1, wherein said administering is peripheral administration or to the central nervous system (CNS).

8. The method of claim 1, wherein said inhibitor inhibits MCP-1.

9. The method of claim 8, wherein said inhibitor is selected from the group consisting of Carlumab, Bindarit and mNOX-E36.

10. The method of claim 1, wherein said inhibitor inhibits CCR2.

11. The method of claim 10, wherein said inhibitor is selected from the group consisting of S0916 (MLN1202), RS504393, PF-4136309 (INCB8761), BMS-813160, BMS-741672 and Cenicriviric (TAK-652, TBR-652).

12. The method of claim 1, wherein said inhibitor inhibits CCR4.

13. The method of claim 1, wherein said inhibitor is an anti MCP-1 antibody.

14. The method of claim 1, further comprising administering or using or including a modulator of an immune modulating molecule.

15. The method of claim 14, wherein said immune inhibitory molecule comprises an immune checkpoint.

16. The method of claim 15, wherein said immune inhibitory molecule is selected from the group consisting of PD-1, PD-L1, P-selectin, P-selectin Ligand-1, CTLA-4, OX-40, IDO, TIGIT, LAG-3 and ICOS.

17. The method claim 14, further comprising administering or using an inhibitor of a cytokine selected from the group consisting of CCL5, GRO-alpha, PAI-1, IL-6 and IL-8/MIP-2.

18. The method of claim 1, further comprising administering or using or including an inhibitor of MEK and/or BRAF pathway.

19. The method of claim 1, further comprising determining an amount of CCR2 and optionally selectin, P-selectin ligand-1, PD and/or PD-L1 in a biological sample of the subject, wherein presence or level of CCR2, selectin, P-selectin ligand-1, PD and/or PD-L1 above a predetermined threshold is an indication for treatment with an inhibitor of an immune inhibitory molecule and optionally an inhibitor of CCR2, selectin, P-selectin ligand-1, PD and/or PD-L1.

20. The method of claim 5, wherein said metastatic brain melanoma is at an early stage comprising micro-metastases as opposed to macro-metastases.
